(19) United States
(12) Patent Application Publication
(10) Pub. No.: US 2006/0014934 A1
(43) Pub. Date: Jan. 19, 2006

(54) Crystal structure of factor Vai and method for identifying blood factor Va modulators

(76) Inventors: Stephen J. Everse, Shelburne, VT (US); Ty E. Adams, Cambridge (GB); Matthew F. Hockin, Salt Lake City, UT (US); Kenneth G. Mann, Grand Isle, VT (US)

Correspondence Address: EDWARDS & ANGELL, LLP, P.O. BOX 55874, BOSTON, MA, 02205, US

(21) Appl. No.: 11/131,091

(22) Filed: May 17, 2005

Related U.S. Application Data
(60) Provisional application No. 60/572,040, filed on May 18, 2004.

(51) Int. Cl. C07K 14/745; G01N 33/48; A61K 38/48

(52) U.S. Cl. 530/384; 702/19

(57) ABSTRACT

The present invention shows the crystal structure of protein C inactivated factor Va (A1-A3-C1-C2) that depicts a novel domain arrangement. The newly disclosed orientation has implications for binding to membranes essential for function. A high-affinity calcium binding site and a copper binding site have been identified, neither of which show a direct involvement in chain association. This structure represents the largest physiologically relevant fragment of factor Va solved to date and provides a new scaffold for generation of models of coagulation factors.

This work was supported by grants from the National Institutes of Health, HL64891 and HL34575 Department of Energy Grant ER45828. The Government has certain rights in the invention. This application claims benefit of U.S. Provisional Application Ser. No. 60/572,040 filed May 18, 2004.

The atomic coordinates and structure factors have been deposited in the protein databank, www.rcsb.org (PDB ID code 1SDD).

### FIELD OF THE INVENTION

The present invention relates to crystals of factor Vai, an inactivated form of factor V and more particularly to the high resolution structure of Vai obtained by x-ray diffraction. The invention further relates to methods of using the crystal structure coordinates and models of the Vai crystal structure to screen and design therapeutic drugs for intervention in biological processes associated with blood coagulation.

### BACKGROUND OF THE INVENTION

Design of new drugs has evolved over the years from laborious synthesis of a few lead compounds that showed some desired or targeted therapeutic activity to present day computer assisted computational design models. Today, libraries of compounds are docked against a model in silico and only compounds that are computationally determined to interact are actually tested in vitro. In addition, combinatorial libraries are also being directly added to the mother liquor of a crystal or used to stabilize a solution containing a crystal. From these crystals a structure is detemined and lead compounds identified from difference fouriers. These compounds are then tested in vitro and/or in vivo to determine a physiological effect. Chemists can then modify these compounds to change their pharmokinetic properties.

In developed countries, the majority of deaths can be directly or indirectly attributed to an imbalance in hemostasis, leading to thrombosis. These thrombi are a natural result of the coagulation cascade, a process characterized by the localized, but “explosive” generation of α-thrombin and the subsequent formation of a platelet-fibrin clot at the site of vascular injury (Mann, et al. (2003) Arterioscler Thromb Vasc Biol. 23, 17-25). Central to this cascade is the catalytic acceleration of each step through the assembly of the vitamin K-dependent enzyme complexes.

The best-studied complex, prothrombinase, is composed of the serine protease factor Xa, the cofactor protein factor Va, and calcium ions on a phospholipid membrane. The formation of this complex accelerates the conversion of prothrombin to α-thrombin by a factor of 3×10⁵ relative to factor Xa alone (Nesheim, et al. (1979) Journal of Biological Chemistry 254, 10952-10962). This rate enhancement is partly a consequence of factor Xa and prothrombin interactions with the membrane, but more importantly the increase is due to interactions with factor Va that alter both the K_{M} and k_{cat} of the reaction process. Factor Va binds tightly to the platelet membrane (K_{d}˜10⁻⁹ M) and serves as the “glue” by increasing the affinity of factor Xa for the membrane by a factor of 10²-10⁵ (Krishnaswamy & Mann (1988) Journal of Biological Chemistry. 263, 5714-5723) and influencing the catalytic efficiency of prothrombin activation by increasing k_{cat}˜3 ×10³) (Nesheim, et al. (1979) Journal of Biological Chemistry 254, 10952-10962).

Factor V has been isolated from both human and bovine plasman. To date it has not been crystallized. Attempts at crystallizing factor Va have also failed; however, the C2 domain from human factor V has been recombinantly expressed and the crystal structure solved (Macedo-Ribeiro, et al. (1999) Nature. 402, 434-9).

Attempts to crystallize factor Va were reported by Everse, et al. (2001), Crystal Structure of Bovine Factor Va. and Adams, et al. (2002), Crystal Structure of Bovine Factor Va. General features of what was reported to be factor Va were later demonstrated to be factor Vai, the inactivation product of factor Va. Unfortunately, only 2-D drawings were presented for the erroneously identified structural model, which lacked sufficient detail to determine binding surfaces or to design inhibitors. Likewise, no information was provided that would allow construction of factor Vai or methods to produce Vai crystals.

Crystals of factor Va were incorrect and actually later determined to be factor Vai. In these reports no details were provided that would allow anyone to reconstruct either the bovine factor Vai structure or reproduce the crystals. A model was presented previously (Everse, et al., 2001) which displayed “two possible locations for the three A domains”. In fact the authors acknowledged that this model could not be correct as shown because “several loop regions overlap and many of the others are lying outside of the density”. In a later publication (Adams, et al., 2002), the authors declared that the presence of the third A domain was not clear, though some unaccounted density was present where the A2 domain was postulated to lie. Furthermore the model did not adequately reflect data showing that the R factor remained at 38%. The authors noted on-going attempts to modify the A domains to better reflect the density using rounds of model building and refinement. All the disclosed models were in the form of 2D ribbon drawings which cannot be used to design inhibitors because they lack the detail required to adequately determine binding surfaces.

Factor V

Produced in hepatocytes, factor V is secreted into the plasma as a single chain composed of six domains (A1-A2-B-A3-C1-C2) that is devoid of coagulant activity (Mann, K. G. & Kalafatis, M. (2003) Blood. 101, 20-30). Activation results in the removal of the B domain and exposure of the factor Xa binding site on factor Va, which leads to assembly of the prothrombinase complex and the subsequent rapid generation of thrombin (Guinto & Esmon (1984) The Journal of Biological Chemistry. 259, 13986-13992). It remains unclear whether the factor Xa binding site is simply masked by the B domain or is formed by conformational changes resulting from its removal.

Overall, the biophysical properties of the prothrombinase complex have been described in detail, yet the structural basis of its interactions has remained elusive. Consequently, an understanding of how factor Va influences the catalytic activity of factor Xa is highly important for deciphering the function of this complex, and to provide key targets for the treatment of hemostatic disorders.

### SUMMARY OF THE INVENTION

The present invention provides a crystalline isoform of bovine factor Vai, which is an inactivation product of factor Va. The crystal structure of factor Vai differs in structural arrangement from the known crystal structure of factor Va and now provides a tool for designing compounds that inhibit or alter the process of blood clotting at the prothrombinase complex level. The crystal structure of factor Vai can also serve as a model for homologous polypeptides such as factor VIIIa, which are associated with thrombin generation.

The structure of bovine factor Vai reveals for the first time the domain organization and the atomic positions of atoms forming its structure. Factor Vai is a physiologically relevant inactivation product of factor Va produced by activated protein C.

In vertebrate hemostasis, factor Va serves as the cofactor in the prothrombinase complex that results in a 300,000-fold increase in the rate of thrombin generation compared to factor Xa alone. Structurally, little is known about the mechanism by which factor Va alters catalysis within this complex.

The invention comprises the determination of a crystal structure of protein C inactivated factor Va (A1-A3-C1-C2) that depicts a novel domain arrangement. The crystal structure reveals a newly discovered orientation that has implications for binding to membranes essential for function. A high-affinity calcium binding-site and a copper binding-site have both been identified. Surprisingly, neither shows a direct involvement in chain association.

The present invention also relates to a process of drug design for compounds which interact with factor Va. The process involves crystallizing factor Vai and resolving the x-ray crystallography data. The data generated from resolving the x-ray crystallography of Vai is applied to a computer algorithm which generates a model of the Vai crystal structure suitable for use in designing molecules that will act as agonists or antagonists to the Va polypeptide. An interative process can be employed whereby various molecular structures are applied to the computer-generated model to identify potential agonists or antagonists of Va. In one embodiment, the process is utilized to identify modulators of active Va, which serve as lead compounds for the design of potentially therapeutic compounds for the treatment of diseases or disorders associated with blood coagulation disorders.

In still another aspect, the present invention relates to a method of identify compounds which are agonists or antagonists of the activity of factor Va by crystallizing factor Vai and obtaining its crystallography coordinates. The crystallography coordinates are then applied to a computer algorithm such that the algorithm generates a model of factor Vai for use in designing molecules that will act as agonists or antagonists to Va. An iterative process is used to apply various molecular structures to the computer-generated model to identify potential agonists or antagonists. The agonist or antagonist is then optionally synthesized or obtained, and contacted with the molecule to determine the ability of the potential agonist or antagonist to interact with the molecule as defined by the structure coordinates of Table 3, or a portion thereof, in a drug-discovery strategy. A potential drug is candidate selected, in conjunction with computer modeling, by performing rational drug design with the three-dimensional structure.

The present invention also relates to a method for determining the three-dimensional structure of a complex of factor Va with a ligand, wherein x-ray diffraction data for crystals of the complex, the set of atomic coordinates of Table 3 (or portions thereof), and coordinates having a root mean square deviation therefrom with respect to conserved protein backbone atoms of not more than about 1.5 A are used to define the three-dimensional structure of the complex.

The present invention also relates to a computer-based device or system for determining at least a portion of the structure coordinates corresponding to the x-ray diffraction data obtained from a molecule or molecular complex. The computer includes a computer-readable data storage medium having a data storage material encoded with machine-readable data. The data include at least a portion of the structural coordinates of Factor Va according to Table 3. The computer also includes a computer-readable data storage medium having a data storage material encoded with computer-readable data including x-ray diffraction data obtained from the molecule or molecular complex; a working memory for storing instructions for processing the computer-readable data; a central-processing unit coupled to the working memory and to the computer-readable data storage medium for performing a Fourier transform of the machine readable data and for processing the computer-readable data into structure coordinates; and a display coupled to the central-processing unit for displaying the structure coordinates of the molecule or molecular complex.

### BRIEF DESCRIPTION OF THE DRAWINGS

FIG. 1A Schematic drawing of the structure of bovine factor Va. The extent and names of the five domains, metal binding sites, and phosphorylation sites are indicated. Dashed lines and outlined fonts depict the A2 domain that is removed in the factor Vai structure.

FIG. 1B Ribbon diagram of bovine factor Vai, indicating the positions of the carbohydrates, and the metals (Ca²⁺ and Cu²⁺). A van der Waals surface representation is shown in the background. Domains throughout all Figures are as as follows: A1, A3, C1, and C2: all structural Figures were prepared using PYMOL (DeLano (2002) in The PyMOL Molecular Graphics System (DeLano Scientific, San Carlos).

FIG. 2: Domain orientation of model of factor Va (PDB 1FV4) (Pellequer, et al. (2000) Thromb Haemost. 84, 849-57) (A); cryoEM structure of factor Villa (Stoilova-McPhie, et al. (2002) Blood. 99, 1215-23)(B); and crystal structure of bovine Vai (C). The sizes and orientation of the ovals were scaled to match the cryoEM C2 domain.

FIG. 3A Stereo images of the metal binding sites in factor Vai, showing the copper binding site in the A3 domain with anomalous density for the copper shown at 3σ. The trigonal planar coordination geometry is shown with dashed lines.

FIG. 3B Shows nearby residues from the A1 domain and the distance to the closest residue. The octahedral coordination geometry is indicated with dashed lines of the calcium binding-site in the A1 domain.

FIG. 4A Potential membrane binding spikes of the C1 (left) and C2 (right) domains. The domains are displayed in similar orientations with respect to the overall β-barrel fold. Residues potentially involved in membrane binding are shown.

FIG. 4B Packing interactions of the tryptophans from spike C2-1 (2050 & 2051) with a hydrophobic pocket in the A3 domain (surface colors hydrophobic=white; polar=black) from a neighboring molecule.

FIG. 5 Overlaid structure of ceruloplasmin (PDB 1KCW, white) on the bovine Va., structure (black). The ceruloplasmin A domain representing the A2 domain is depicted as a surface representation. Measurements do not include extended loops. Right panel has been rotated 90° about a vertical axis.

FIG. 6 lists the atomic coordinates of the three dimensional factor Vai crystal.

### DETAILED DESCRIPTION OF THE INVENTION

The present invention may be understood more readily by reference to the following detailed description of preferred embodiments of the invention and the figures.

The cofactor protein factor V is directly involved in regulating the production of α-thrombin to maintain vascular integrity and hemostasis. When activated, factor Va interacts with the enzyme, factor Xa, to form the prothrobinase complex on a membrane surface and convert prothrombin to α-thrombin. Once sufficient α-thrombin has been generated, factor Va is inactivated by the anticoagulant protein APC, to form factor Vai.

The crystal structure of factor Vai at 2.8 Å resolution using a combination of MIRAS and molecular replacements has been determined. The crystals are orthorhombic, in space group P212121 with cell dimensions of a=63.37, b=86.56, and c=229.20 Å. Composed of two ceruloplasmin-like A domains and two discoidin-like C domains, the overall structure resembles a distorted butterfly where the A domains form the upper wings and both of the C domains form the lower wings and can interact with a membrane surface.

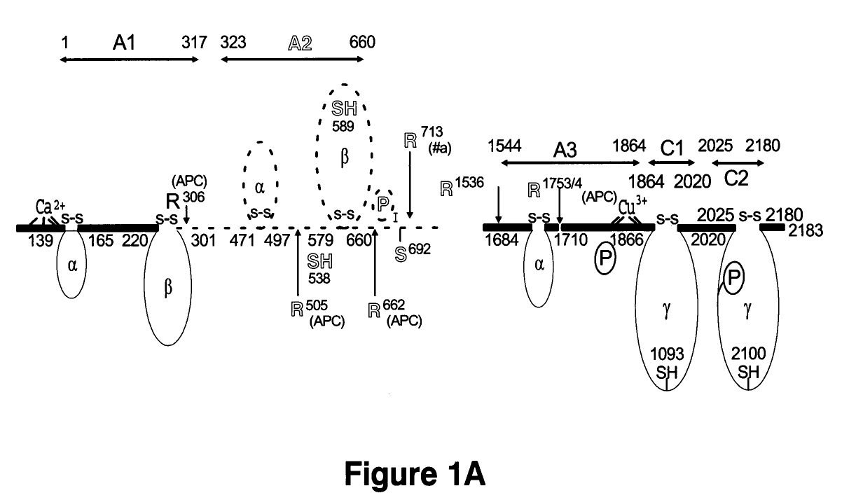

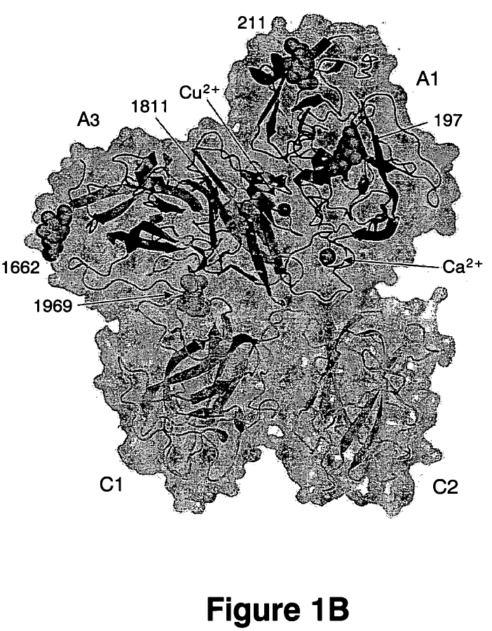

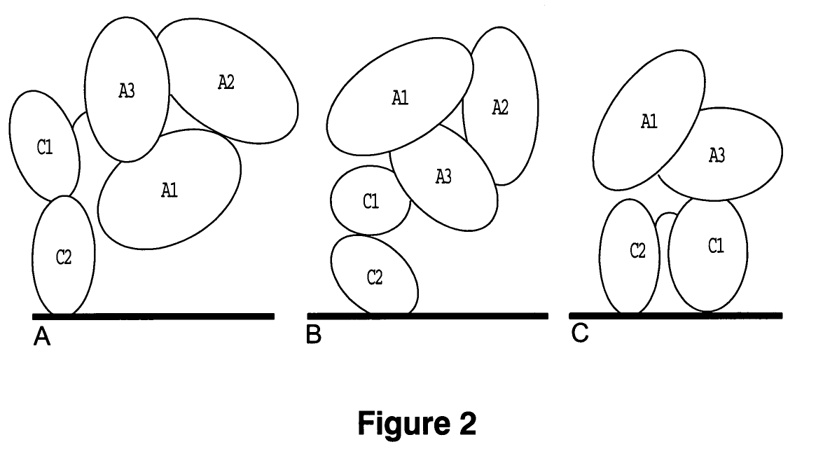

One of the key reactions in down-regulating coagulation is the inactivation of factor Va by the anticoagulant activated protein C (APC) (Walker, et al. (1979) Biochimica et Biophysica Acta. 571, 333-342). APC cleaves at Arg-505 and Arg-306, leading to the spontaneous release of the A2 domain and a complete loss of cofactor activity (Kalafatis & Mann (1993) Journal of Biological Chemistry. 268, 27246-27257). The remaining fragment, factor Va, is composed of the A1 domain non-covalently associated with the light chain (FIG. 1A). Individuals carrying mutations in factor V at any of the APC cleavage sites, such as factor V^{Leiden}, have an increased risk of thrombosis due to incomplete inactivation of factor Va.

Factor V shares strong functional and sequence homology with factor VIII (anti-hemophilic factor). Both have an identical domain organization with the B domains that act as large activation peptides (comprising nearly half of each pro-cofactor) with no detectable homology either to each other or to any other known protein. The A domains (˜330 AA) of factors V and VIII share approximately 40% sequence identity with each other and roughly 30% with the A domains of ceruloplasmin (Gitschier, et al. (1984) Nature. 312, 326-330). The C domains (−150 AA) of factors V and VIII are approximately 43% identical and have no strong homology to any other known proteins. There is a weak homology with the discoidin-like proteins, a family proteins involved in cell adhesion (Fuentes-Prior, et al. (2002) Curr Protein Pept Sci. 3, 313-39). Recent structures of recombinant C2 domains from both factor V and factor VIII are consistent with those observed in other discoidin domain containing proteins (Pratt, et al. (1999) Nature. 402, 439-42).

Membrane binding of factor Va is mediated through interactions involving the light chain. Specifically these interactions have been localized to the C2 domain (Ortel, et al. (1994) The Journal of Biological Chemistry. 269, 15989-15905). Antibodies to the C2 domain of both factors V and VIII have been shown to interfere with membrane binding and inhibit cofactor function. Deletion of the entire C2 domain results in a complete loss of phosphatidylserine-specific membrane binding. Alanine scanning mutagenesis within the C2 identified several key polar and hydrophobic amino acids as necessary for achieving maximal cofactor function (Nicolaes, et al. (2000) Blood Coagul Fibrinolysis. 11, 89-100).

The 2.8 Å crystal structure of factor Vai has now been determined, revealing a domain arrangement that predicts a more extensive membrane binding. Identification of the high-affinity calcium binding site as well as the location of a copper ion suggests a possible mechanism for heavy and light chain association. Using this information, development of new paradigms for the function of these cofactors in vivo is possible.

The newly determined Vai structure represents the largest physiologically relevant fragment of factor Va solved to date and provides a new scaffold for the future generation of models of coagulation cofactors. The crystalline inactivated form of the factor Va structure is contrary to previous electron microscopy and homology models that suggested the C domains in factor Va are stacked upon each other. This difference in C domain alignment, along with others revealed in the crystal structure of the inactivation product, provides a new foundation for understanding the role of factor V in regulating the formation of blood clots.

Thus, the crystal structure of inactivated Va for the first time integrates a wealth of experimental data, so that a new tool is provided not only for determining the mechanisms involved in the prothrombinase complex function but also for rational design of new drug compounds for intervention in the blood clotting process.

Phrombinase Complex. The best studied complex of the coagulation cascade is prothrombinase, i.e., protease factor Xa and the cofactor Va with calcium ions and anionic membrane, which results in a 300.000 fold increase in catalytic efficiency compared to Xa alone (Nesheim, et al. (1979) The Journal of Biological Chemistry. 254, 508-517). Factor Va is produced from a single chain in native cofactor (A1-A2-B-A3-C1-C2) that is activated by thrombin with release of the B domain. Factor Va is cleaved by the anticoagulant activated protein C (APC) at three sites leading to the spontaneous release of the A2 domain and complete inactivation resulting in factor Vai.

The present invention comprises the solution of the crystal structure of bovine factor Vai to a resolution of 2.8 Å. The crystal structure shows that the C1 and C2 domains are side by side, implying that both of these domains can interact with membrane surface. The A domains rest upon a platform created by the C domains. Within the A domains, binding sites for copper and calcium have been identified. In addition, 5 of the 7 potential glycosylation sites are observed. This structure is inconsistent with previous models of factor Va based upon the structures of ceruloplasmin and the C2 domains from either factor V or factor VIII, suggesting that the C domains are stacked upon each other and also showing the orientation of the A domains with respect to the overall architecture. The structural model of factor Vai provides a foundation for the complete model of factor Va and its interaction with factor Xa.

Domain Structure and Organization. The bovine Va, structure is composed of two of the three A domains from factor V (A1 & A3) and both C domains (C1 & C2) (FIG. 1A). Each A domain is comprised of two linked cupredoxin-like (3-barrels and shares high structural conservation with each other and the three A domains of ceruloplasmin (root mean-square deviation (rmsd) between 0.98-1.37 Å for 268 Cα atoms) (Zaitseva, et al. (1996) Journal of Biological Inorganic Chemistry. 1, 15-23). A single metal ion is observed within each A domain and the site is distinct from the metal binding sites found in ceruloplasmin. The factor Vai, C domains can be described as a distorted jelly-roll (β-barrel with a high degree of structural similarity between the C1 and C2 (rmsd 0.96 Å for 157 Cα atoms). The structure of these is very similar to the recombinant C2 structures of human factors V and VIII (rmsd 0.61-0.87 Å for 159 Cα atoms) (Pratt, et al. (1999) Nature. 402, 439-42).

One of the most exciting aspects of the Vai crystal structure is the unique domain arrangement (FIG. 1B). Consistent with earlier models, the A1 and A3 domains are arranged around a pseudo-three-fold axis similar to that observed in ceruloplasmin. Several disordered loops are not visible in the structure, including residues flanking the additional bovine APC cleavage site found within the A3 domain. Within the A1 domain, the disordered loops are localized along one edge of the domain and may be due to partial destabilization of the domain caused by the removal of the A2 domain. Looking down the three-fold axis within the A domains, the C domains are aligned “edge-to-edge” forming a platform upon which the A domains rest. This is completely different from models in which the C1 was predicted to be stacked above the C2 domain (FIG. 2). The crystal structure of Vai shows that the C domains are side-by-side, indicating that both domains are likely to be of high importance in membrane binding.

Domain Interfaces. The interface between the C1 and C2 domains buries less than 700 Q 2 of surface area and contains neither a substantial electrostatic nor hydrophobic character. In fact, only three hydrogen bonds exist between the two domains, two of which occur within the four amino acid linker between the disulfide bonds in the C 1 (Cys-1866-Cys-2020) and C2 (Cys-2025-Cys-2180) domains. This, in conjunction with a hydrogen bond between Asp-1863 in the A3 domain and Ser-2026 in the C2 domain, may restrain the linker between the C1 and C2 domains thereby restricting the orientation of the C2 domain with respect to the rest of the molecule.

The interface between the C1 and A3 domains contains both hydrophobic and electrostatic interactions that bury 1758 Å² of surface area. One end of the interface is anchored by hydrophobic interactions between residues from the A3 domain (Leu-1860 and Val-1862) and the C1 domain (Leu-1931, Val-1996 and Val-2022). The other end of the interface predominantly involves hydrogen bonds and salt bridges between a loop (Phe-1966-Val-1974) that interrupts β-strand in the C1 domain (Asn-1962-Asn-1980) and charged residues within the A3 domain.

In contrast, the A1 domain does not substantially interact with the C2 domain. Whether this is physiologically relevant or the result of relaxation of the domain due to the excision of the A2 domain is unclear and will depend on determination of a factor Va structure. This may also explain why the A1 domain has the highest average β-factors among the 4 domains. The lack of interactions between the A1 and C2 domains may indicate that the association between the A1 domain and light chain is entirely mediated via interactions with the A3 domain. A network of hydrogen bonds dispersed throughout the entire 2662 Å² of buried surface area is observed within this reciprocally contoured surface.

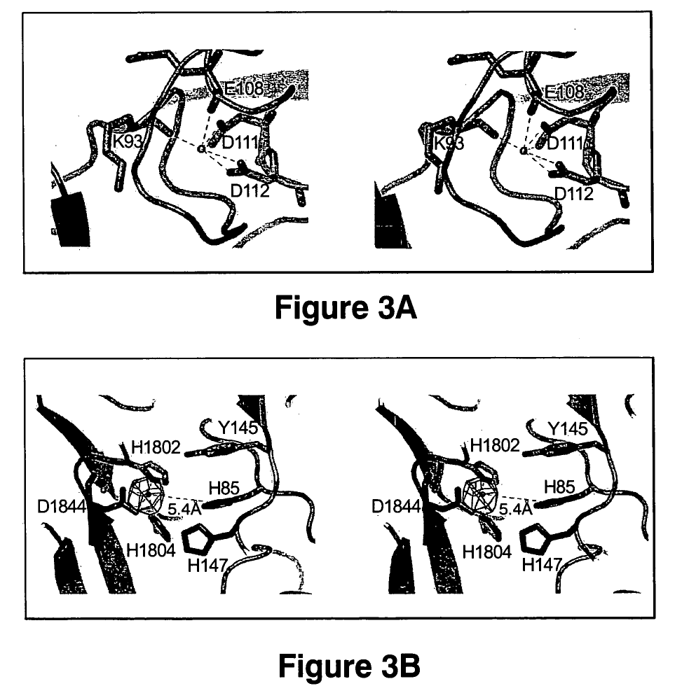

Metal Binding Sites. The anomalous signal for a copper ion within the buried surface between the A1 and A3 domains can be clearly observed (FIG. 3A). Experimental evidence has demonstrated that both factor V and VIII bind a single copper atom. A functional role for copper in factor V or Va has not yet been ascertained, but in factor VIII, a type II copper leads to approximately 100-fold affinity between the factor VIII subunits. FIG. 3A shows ligands to the Cu²⁺ include: His-1802, His-1804 (both predicted), and Asp-1844 in a trigonal planar coordination geometry. Although homology modeling predicted that a Cu²⁺ in factor Va would bridge the heavy and light chains (Villoutreix & Dahlback (1998) Protein Sci. 7, 1317-25), the metal in the structure shown is more than 5 Å from any potential ligand in the A1 domain. Therefore this copper ion may have a structural role in providing additional stabilization of the A1-A3 interface rather than directly linking the two domains.

Chain association is required for factor Va function and has been shown to be dependent on a divalent cation (Krishnaswamy, et al. (1989) Journal of Biological Chemistry. 264, 3160-3168). Factors V and Va contain a single high-affinity Ca²⁺ site as well as several low-affinity sites. The occupancy of the high-affinity site is essential for the interaction of the heavy and light chains and the subsequent activity of factor Va. Historically, this Ca²⁺ was believed to bridge the heavy and light chains; however, the factor Va, structure clearly reveals that the Ca²⁺ is entirely coordinated by ligands in the A1 domain (FIG. 3B). These ligands include the side chains of both Asp-111 and Asp-112, along with the main chain carbonyl oxygens of Lys-93 and Glu-108. Recent mutational data support a role for Ca²⁺ binding in both factors Va and VIIIa at this site (Zeibdawi, et al. (2004) Biochem J. 377, 141-148).

Since chain association cannot be directly attributed to the coordination of Ca²⁺, it is likely that the loop comprising Lys-93-Asp-112 adopts a conformation that results in several essential interactions between the A1 and the A3 domains. For example, the carboxylate side chain of Glu-96 forms a hydrogen bond with His-1804 in the A3 domain, and the terminal amino group of Lys-93 forms a hydrogen bond to the backbone carbonyl of Trp-1840. These interactions, along with a hydrophobic stacking of Tyr-100 and Leu-1842, suggest that disruption of the Ca²⁺ binding loop may interfere with the packing of the A3 domain against the A1 domain which may be sufficient to force the dissociation of the heavy and light chains of factor Va.

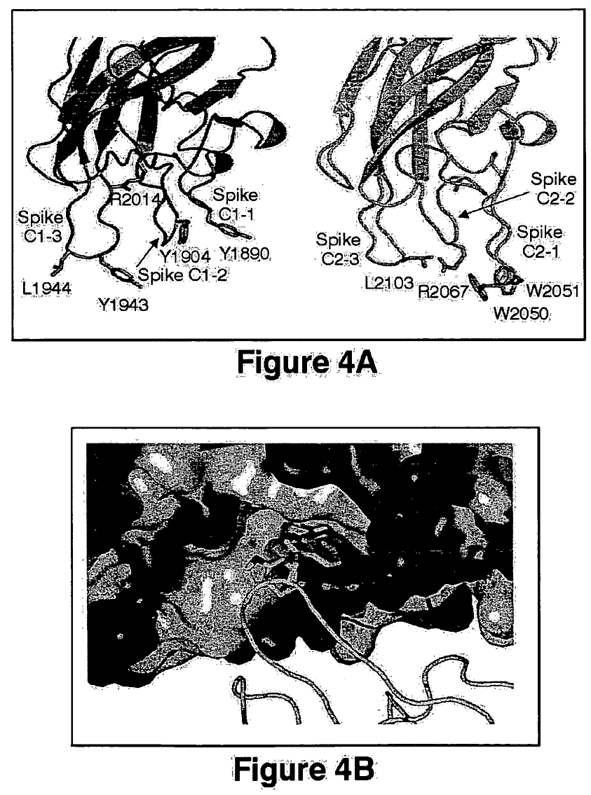

Membrane Interactions. Protruding from the bottom of the β-sandwich in each C domain are three β-hairpin loops, referred to as “spikes”, that form a pocket lined with both hydrophobic and polar amino acids (FIG. 4A). Factor Vai., spike C2-1 (Ser-2045-Trp-2055) contains two tryptophans (Trp-2050 and Trp-2051) at its apex extending away from the pocket. Macedo-Ribeiro and co-workers (Macedo-Ribeiro, et al. (1999) Nature. 402, 434-9) identified two crystal forms of the recombinant factor V C2 domain in which this spike moved by 7 Å. They hypothesized that this movement resulted in the exposure of the phospholipid binding pocket and allowed membrane binding. In the disclosed factor Vai, structure, these tryptophans are constrained by crystal packing interactions with an A3 domain from a neighboring molecule (FIG. 4B), burying them into a hydrophobic cleft on the A3 domain. On the other hand, in factor Va, this cleft may be masked by interactions with the A2 domain, yet these tryptophans clearly have a high propensity for inserting into a hydrophobic environment. In agreement with other studies on factor Va, these tryptophans are the most likely point of lipid bilayer insertion during membrane binding of the C2 domain. However, conclusions regarding the physiological role of the movement of this loop with respect to membrane interaction awaits a structure with bound lipid.

Given the position of the C1 domain relative to the C2 domain, it also has the potential to interact with the membrane. Like the C2 domain, the C1 domain contains three spikes, although one spike (C1-1: Glu-1886-Trp-1891) contains a 5-residue deletion, eliminating the two putative membrane-inserting tryptophans. Nevertheless at the apex of spike C1-3 (Gly-1939-Tyr-1948), Leu-1944 is exposed to solvent and in position to insert into the membrane. The C1 spikes also contain several tyrosine residues (Tyr-1890, C1-1; Tyr-1904, C1-2; Tyr-1943, C1-3) located at or near the apex of each loop. Unlike the tryptophans on the C2 spikes, the tyrosines would not insert into, but rather could interact favorably with, phospholipid membranes (Rinia, et al. (2002) Biochemistry. 41, 2814-24).

A recent report using alanine-scanning mutagenesis identified these leucine and tyrosine residues on the C1-3 spike as important in prothrombinase activity (Saleh, et al. (2004) Thromb Haemost. 91, 16-27). Additionally, two arginine residues in human factor Va (Lys-2010 and Arg-2014 in the bovine molecule) have been shown to have a significant impact on function. In the Vai structure these particular residues are solvent exposed, lie on opposite sides of the domain, and could potentially interact with negatively charged phospholipid head groups on the membrane surface.

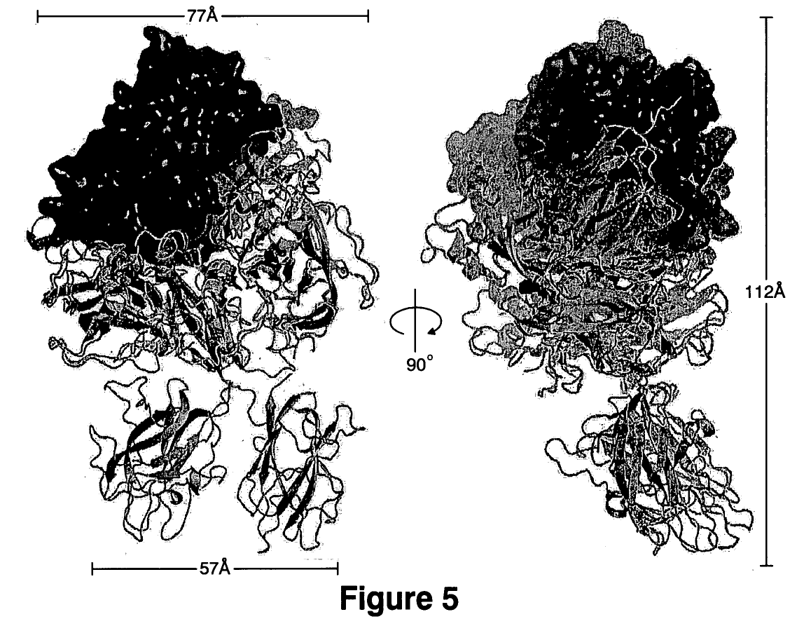

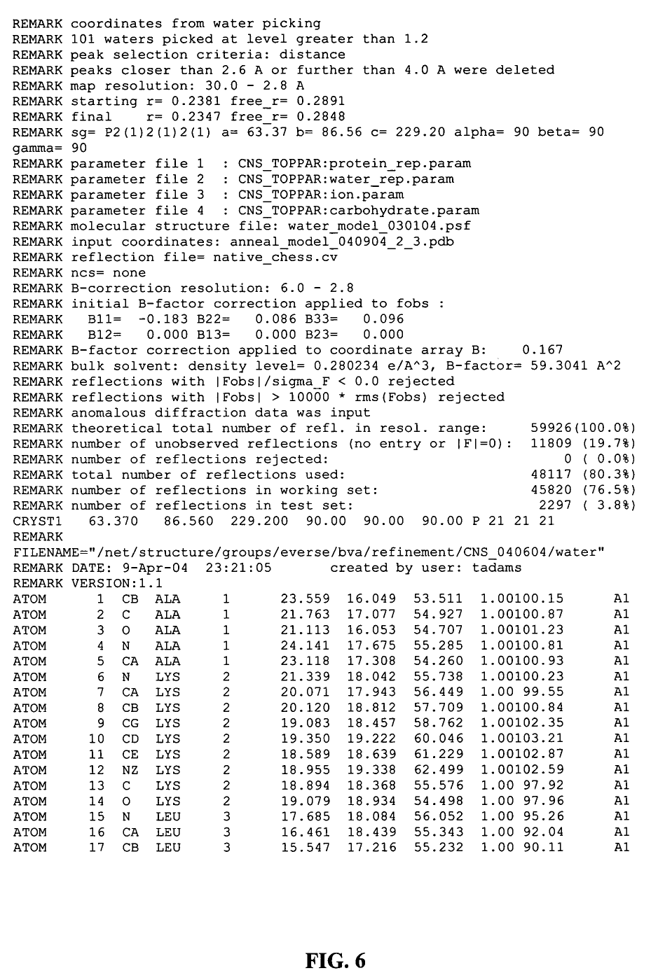

Structure Validation. Although a structural rearrangement due to APC inactivation cannot be completely ruled out, several pieces of evidence argue against this possibility. First, reconstructions of factor Va using electron microscopy (EM) depict a molecule extending −100 Å from the cell membrane and these dimensions correlate well with the more recent 15 Å EM projection structure of factor VIIIa (Stoylova, et al. (1999) J. Biol Chem. 274, 36573-8). In homology models of factors Va and VIIIa based on these EM data, a variety of domain orientations have been proposed (FIG. 2). Most notably the C1 domain was predicted to stack upon the C2 domain vertically outward from the membrane, thereby lifting the A domains to a height appropriate for interaction with its specific enzyme partner, factors Xa and IXa respectively. The structure of Vai shown in FIG. 2C has dimensions similar to the EM derived values with the differences attributed to the missing A2 domain. Second, overlaying ceruloplasmin on the A1 and A3 domains (rmsd 1.3 Å for 544 Ca²⁺ atoms) places the missing A domain exactly between them, without overlap (FIG. 5). The addition of this A domain representing the A2 domain of factor Va increases the height of the structure to 112 Å, well within the experimental error of the EM measurements. Third, FRET (fluorescence resonance energy transfer) data predict that the APC active site is 94 Å above the surface of the membrane. Inspection of the APC cleavage site (Arg-505) in the potential A2 domain, reveals that it lies approximately 90 Å above the putative membrane surface, whereas when the C domains are stacked on top of one another this site is only 75 Å above the membrane surface.

The structure of factor Vai, answers several important questions regarding factor Va function, including metal disposition, chain association, and membrane binding. It has been demonstrated that the Ca²⁺ is coordinated completely within the A1 domain and neither Ca²⁺ nor Cu²⁺ plays a direct role in chain association. Ca²⁺ may order a critical loop within the A1 domain to allow for constructive interactions between the A1 and A3 domains. This hypothesis is supported by mutational studies of residues within this loop as exemplified by the E96A mutation in factor Va, where the two chains remain associated in the presence of Ca²⁺ yet show a reduced cofactor activity (Zeibdawi, et al. (2004) Biochem J. 377, 141-148). In the factor Vai structure, Glu-96 does not participate in Ca²⁺ binding, but instead interacts with the A3 domain. Additionally, removal of the copper ion results in no loss of factor Va cofactor function within the prothrombinase complex. Since no particular function is attributed to copper binding, it may simply be a remnant of the cupredoxin-like protein fold.

The placement of the C domains adjacent to one another provides a platform that lifts the A domains to a height above the membrane surface appropriate for interaction with their physiologic partners (factor Xa, prothrombin, APC). The results disclosed herein, in combination with recent mutational studies, indicate that both C domains may contribute to the factor Va binding to the membrane surface. Membrane binding may be initiated by the C2 domain. The structural flexibility between this domain and the rest of the molecule would then allow the C1 domain to locate its cognate lipid within the membrane, thereby strengthening the overall affinity of factor Va for the platelet surface.

Due to its high degree of functional and structural homology to factor Va, the structure of factor Va, provides a basis for construction of a model of factor VIIIa. Since factor VIII deficiency is the causative agent of hemophilia A, modeling studies will be enhanced by the rich database of clinically relevant factor VIII mutations and provide a more coherent approach to the design of pharmaceuticals for the treatment of hemophilia as well as other thrombotic disorders.

Definitions:

Abbreviations: APC, activated protein C; rmsd, root mean square deviation

As used throughout the specification, it is to be understood that factor Vai is an inactivation product of factor Va and does not have the same crystal structure as Va.

The term “a” as used herein is not intended to limit any of the disclosed or claimed subject matter.

Crystals of factor Vai can be grown by a number of techniques including batch crystallization, vapor diffusion (either by sitting drop or hanging drop) and by microdialysis. Seeding of the crystals in some instances is required to obtain X-ray quality crystals. Standard micro and/or macro seeding of crystals may therefore be used. In addition, the crystals can be grown at a variety of temperatures with only a slight modification of the initial protein concentration and/or PEG concentration.

Once a crystal of the present invention is grown, X-ray diffraction data can be collected. Crystals can be characterized by using X-rays produced in a conventional source such as a sealed tube or a rotating anode or using a synchrontron surce. X-ray diffraction data can be collected using, for example, a MAR imaging plate detector and/or a CCD based detector.

Data processing and reduction can be carried out using programs such as HKL, DENZO, and SCALEPACK (Otwinowski & Minor (1997) in Methods in Enzymology, Part A, eds. Carter, C. W. & Sweet, R. M. (Academic Press, San Diego), 276, 307-326). In addition, X-PLOR, or CNS may be utilized for bulk solvent correction and B-factor scaling. Electron density maps can be calculated using fft in the CCP4 package or routines within X-PLOR or CNS. Molecular models can be built into this map using O (Jones, et al., ACM Crystallogr. A47:110-119 (1991), XTALVIEW or QUANTA96. Refinement can be done using CNS or REFMAC free R-value to monitor the course of refinement.

Once the three-dimensional structure of a crystal comprising factor Vai is determined, a potential ligand (antagonist or agonist) is evaluated through the use of computer modeling using a docking program such as FelxiDock (Tripos, St. Louis, Mo.), GRAM (Medical Univ. Of South Carolina), DOCK3.5 and 4.0 (Univ. Calif. San Francisco), Glide (Schrodinger, Portland, Oreg.), Gold (Cambridge Crystallographic Data Centre, UK), FLEX-X (BioSolvelT GmbH, Germany); AGDOCK, Hex, FTDOCK, or AUTODOCK (Scripps Research Institute). This procedure can include computer fitting of potential ligands to a selected substrate-binding domain to ascertain how well the shape and the chemical structure of the potential ligand will complement or interfere with the factor Va substrate-binding regions. Computer programs can also be employed to estimate the attraction, repulsion, and steric hindrance of ligands to such a region. Generally the tighter the fit (e.g., the lower the steric hindrance, and/or the greater the attractive force) the more potent the potential drug will be since these properties are consistent with a tighter-binding constant.

“Binding domain” also referred to as “binding region”, “binding cleft”, “substrate-binding site catalytic domain,” or “substrate-binding domain,” all refer to a region or regions of a molecule or molecular complex, that, as a result of its shape, can associate with another chemical entity or compound. Such regions are of significant utility in fields such as drug discovery. The association of natural ligands or substrates with binding regions of their corresponding receptors or enzymes is the basis of many biological mechanisms of action.

Similarly, many drugs exert their biological effects via an interaction with the binding clefts of a receptor or enzyme. Such interactions may occur with all or part of the binding cleft. An understanding of such interactions can lead to the design of drugs having more favorable and specific interactions with their target receptor or enzyme, and thus, improved biological effects. Therefore, information related to ligand binding with a factor Va substrate-binding region is valuable in designing potential modulators of factor Va. Further, the more specificity in the design of a potential drug the more likely that the drug will not interact with other similar proteins, thus, minimizing potential side effects due to unwanted cross interactions.

Initially, a potential ligand can be identified by screening a random chemical and/or small molecule library. A ligand selected in this manner is then be systematically modified by computer-modeling programs until one or more promising potential ligands are identified. Such analysis has been shown to be effective in the development of HIV protease inhibitors. Such computer modeling allows the selection of a finite number of rational chemical modifications, as opposed to the countless number of essentially random chemical modifications that could be made, and of which any one might lead to a useful drug. Each chemical modification requires additional chemical steps, which while being reasonable for the synthesis of a finite number of compounds, quickly becomes overwhelming if all possible modifications needed to be synthesized. Thus, through the use of the model coordinates disclosed herein and computer modeling, a large number of these compounds can be rapidly screened in silico, and a few likely candidates can be identified without the laborious synthesis of untold numbers of compounds.

Once a potential ligand (agonist or antagonist) is identified it can be either selected from commercial libraries of compounds or alternatively the potential ligand may be synthesized de novo. The de novo synthesis of one or even a relatively small group of specific compounds is reasonable in the art of drug design. The prospective drug can be tested in a suitable binding assay to test its ability to bind to the Va substrate binding region. The effect of the prospective drug on factor Va activity can also be determined using assays known in the art.

When a suitable compound is identified, a supplemental crystal can be grown which comprises a protein ligand complex formed between the factor Vai domain and the compound by co-crystallization. In addition, the compound may also be soaked into existing crystals. Preferably the crystal effectively diffracts X-rays allowing the determination of the atomic coordinates of the protein-ligand complex to a resolution value of at least 2.8 Å or less, more preferably about 2.0 Å or less. Molecular replacement can be used to determine the three-dimensional structure of such a supplemental crystal.

Molecular replacement involves using a known three-dimensional structure as a search model to determine the ligand complex in a new crystal form. The measured X-ray diffraction properties of the new crystal are compared with those calculated from a search model structure to compute the position and orientation of the protein in the new crystal. Computer programs that can be used for this purpose include: CNS, CCP4, X-PLOR, EPMR, and AMORE. Once the position and orientation are known an electron density map can be calculated using the search model to provide X-ray phases. Thereafter, the electron density is inspected for structural differences and the search model is modified to conform to the new data. Using this approach, it is possible to use the factor Vai structure to solve the three-dimensional structures of any such factor Vai polypeptide-ligand complex.

Further refinements to the structure of the drug will generally be necessary and can be made by the successive iterations of any and/or all of the steps provided by the methods and procedures discussed.

Structure coordinates generated from a factor Vai-ligand complex may be used to generate a three-dimensional shape. This is achieved through the use of commercially available software that is capable of generating three-dimensional graphical representations of molecules or portions thereof from a set of structure coordinates.

Materials and Methods

Bovine factor Va was purified using a modified procedure (Nesheim, et al. (1981) in Methods in Enzymology, Proteolytic Enzymes, Part C., ed. Lorand, L. (Academic Press Inc., New York), pp. 249-285). Bovine activated protein C (APC) was a generous gift from Haematologic Technologies (Essex Junction, Vt.).

Data for this study were measured at beamline X12C & X25 of the National Synchrotron Light Source and upon data collected at CHESS using the Macromolecular Diffraction at CHESS (MacCHESS) facility.

Data Processing and Structure Refinement All diffraction datasets were processed using DENZO and individual datasets were scaled and merged using SCALEPACK (Otwinowski & Minor (1997) in Methods in Enzymology, Part A, eds. Carter, C. W. & Sweet, R. M. (Academic Press, Szn Diego), 276, 307-326). All data were subsequently scaled to the native data using SCALEIT (Collaborative Computational Project, N. (1994) Acta Crystallographica D Biological Crystallography D50, 760-763) and heavy atom sites were determined by SOLVE (Terwilliger & Berendzen, J. (1999) Acta Crystallographica D Biological Crystallography. 55, 849-61). Heavy-atom refinement and phasing was carried out using the maximum likelihood program MLPHARE in the CCP4 program suite (Collaborative Computational Project, N. (1994) Acta Crystallographica D Biological Crystallography D50, 760-763). A single round of density modification using SOLOMON (Abrahams & Leslie (1996) Acta Crystallogr D Biol Crystallogr 52, 30-42) was followed by additional heavy-atom refinement and phasing yielded phase estimations at 3.7 Å with a final figure of merit of 0.83. The resulting map was not immediately interpretable. The partial phase information was used in a molecular replacement search using the 6D phased rotation/translation program BRUTEPTF* with the previously solved factor V C2 domain (PDB 1CZT) and factor Va A1 domain model (PDB 1FV4) as search models.

### EXAMPLES

### Example 1

The search results yielded two unique A domain solutions with correlation coefficients of 0.198 and 0.186 as well as two unique C domain solutions with correlation coefficients of 0.249 and 0.217. Model phases combined with experimental phases produced interpretable density allowing for manual model fitting and rebuilding of the molecular replacement solution. The structure was refined with alternating rounds of refinement including simulated annealing using CNS (Brunger, et al. (1998) Acta Crystallographica D Biological Crystallography. 54, 905-21) and model rebuilding in 0 (Jones, et al. (1991) Acta Crystallographica. A47, 110-119) (Table 1).

### Example 2

### Inactivation of Bovine Factor Va by Bovine APC

Bovine factor Va (40 μM) was extensively dialyzed against 20 mM HEPES, 150 mM NaCl, 2 mM CaCl², pH 7.4 (HBSCa). Factor Va was incubated with 100 μM phospholipid vesicles (75% phosphatidylcholine: 25% phosphatidylserine) at 37° C. for 1 hour. Bovine APC was added (250 nM) and the sample was incubated at 37° C. for 3 hours. Factor V activity was monitored by single-stage clotting assays. The sample was loaded onto a Poros HQ20 (4.6×100 mm) equilibrated in 20 mM HEPES, 2 mM CaCl₂ and eluted with a gradient elution of 0 to 500 mM NaCl in equilibration buffer over 10 minutes. Fractions identified by SDS-PAGE as containing A2-domainless factor Va., were pooled and analyzed for residual factor Va activity. Purified protein was stored in HBS-Ca at −20° C.

### Example 3

### Crystallization and Data Collection

Purified bovine factor Va., in 20 mM HEPES, 150 mM NaCl, 2 mM CaCl₂ (pH 7.4) was crystallized at ˜6.5 mg/mL by the vapor diffusion sitting-drop method at 12° C. against 200 mM MgCl₂, 16% PEG 3350 (pH 5.0). After 521 days, diffraction quality crystals appeared (Table 1). Three isomorphous heavy atom derivative crystals were identified from native crystals soaked in mother liquor containing either 10 mM tetrakismercuroxymethane (TAMM), 10 mM ethylmercury (EtHg) or 2.5 mM lead acetate (PbAc) Supporting data are shown in Table 2.

TABLE 1
Data Collection and Refinement Statistics

| | Native^{b} |
|---|---|
| resolution limits (Å) | 30-2.8 |
| Space group | P212121 |
| Cell dimensions (Å) | a = 63.37 |
| | b = 86.56 |
| | c = 229.20 |
| Reflections | 30822 |
| completeness (%)^{a} | 97.4 (94.9) |
| Redundancy | 3.6 |
| I/o^{a} | 16.9 (4.1) |
| R_{syn t} (%)^{a} | 6.9 (29.4) |
| model details | 7012 |
| no. protein atoms | |
| no. solvent molecules | 390 |
| additional ligands | 5 NAG, 1Cu²⁺, |
| | 1Ca²⁺ |
| Average B-factor (A²) | |
| protein main-chain | 46.2 |
| protein side-chain | 47.3 |
| solvent molecules | 50.9 |
| Rfactor (Rfee) (%) | 23.3 (29.2) |
| Rms deviation from ideal geometry | |
| bond lengths (A) | 0.008 |
| Bond angles (deg.) | 1.412 |
| Residues in allowed Ramachandran regions (%) | 98.5 |

^{a}Data in parenthesis represent the highest resolution shell 2.90-2.80 A.
^{b}Collected at CHESS A-1 beamline (.=0.935 A) using an ADSC Quantum-210 CCD detector.

TABLE 2
Data Collection and Phasing Statistics for Derivatives
TAMM^{C}

| | Inflection | Peak | Remote | PbAc^{d} | EtHg^{d} |
|---|---|---|---|---|---|
| resolution limits | | | | | |
| (A) space group | 30-3.5 | 30-3.5 | 30-3.6 | 30-4.0 | 30-3.65 |
| resolution limits (Å) | | | | | |
| cell dimensions (A) | | | | | |
| space group | P2₁2₁2₁ | P2₁2₁2₁ | P2₁2₁2₁ | P2₁2₁2₁ | P2₁2₁2₁ |
| cell dimensions (Å) | a = 63.175 | a = 63.251 | a = 63.291 | a = 63.396 | a = 63.195 |
| | b = 86.209 | b = 86.319 | b = 86.374 | b = 87.272 | b = 86.957 |
| | c = 228.187 | c = 229.039 | c = 229.170 | c = 229.354 | c = 230.016 |
| unique reflections | 23615 | 23653 | 23772 | 11346 | 13158 |
| mosaicity (deg) | 0.72 | 0.71 | 0.72 | 0.72 | 0.69 |
| R_{sym} (%)^{a} | 4.3 (23.1) | 4.3 (21.4) | 4.5 (23.9) | 8.0 (18.3) | 7.1 (13.1) |
| redundancy | 13.5 | 13.1 | 13.1 | 12.7 | 7.2 |
| I/σ^{a} | 24.7 (12.7) | 24.8 (16.5) | 23.2 (11.4) | 23.8 (10.8) | 16.5 (10.1) |
| completeness (%)^{a} | 90.2 (79.4) | 90.6 (79.8) | 89.3 (78.4) | 95.3 (88.1) | 91.6 (92.0) |
| wavelength (Å) | 1.0106 | 1.0097 | 0.9803 | 1.100 | 1.100 |
| no. of sites | 3 | 3 | 3 | 2 | 2 |
| R_{cullis}^{b} | 0.52 (0.65) | 0.52 (0.68) | 0.54 (0.70) | 0.86 (0.88) | 0.84 (0.91) |
| phasing power^{b} | 1.49 (2.71) | 1.42 (2.69) | 1.38 (2.56) | 0.65 (0.69) | 0.49 (0.56) |
| phasing resolution (Å) | 3.7 | 3.7 | 3.7 | 4.2 | 3.8 |

^{a}Data in parenthesis represent outermost shell statistics.
^{b}Data in parenthesis represent acentric reflection statistics.
^{c}Collected at Brookhaven National Laboratory NSLS beamline X12C using a Brandeis B1.2 CCD detector.
^{d}Collected at Brookhaven National Laboratory NSLS beamline X25 using a Brandeis B4 CCD detector.

The 2.8 Å crystal structure of bovine factor Vai contains 871 amino acids, 333 water molecules, a calcium atom, a copper atom, and 5 carbohydrates.

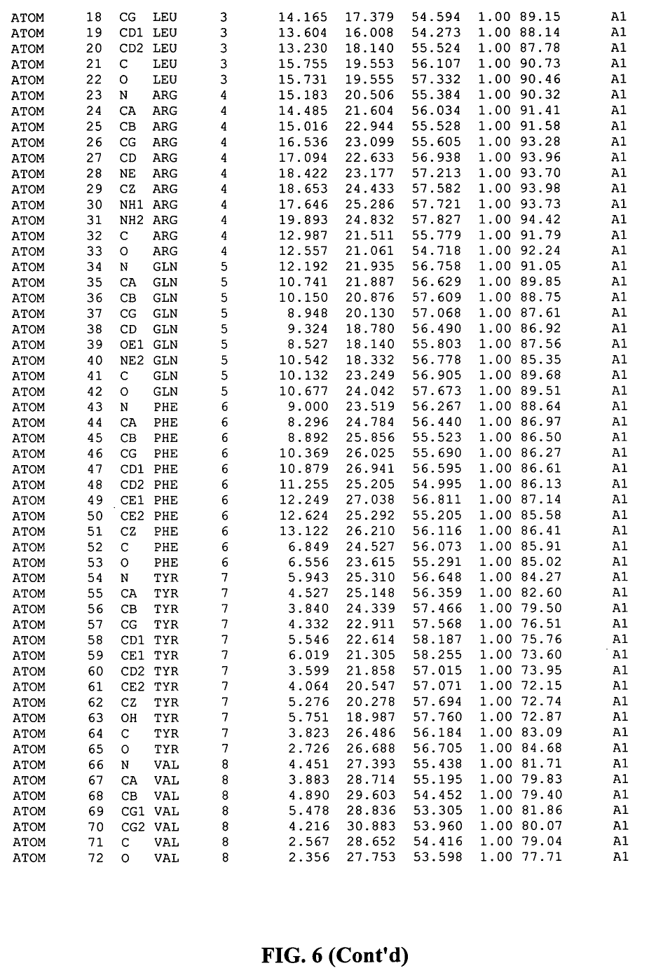

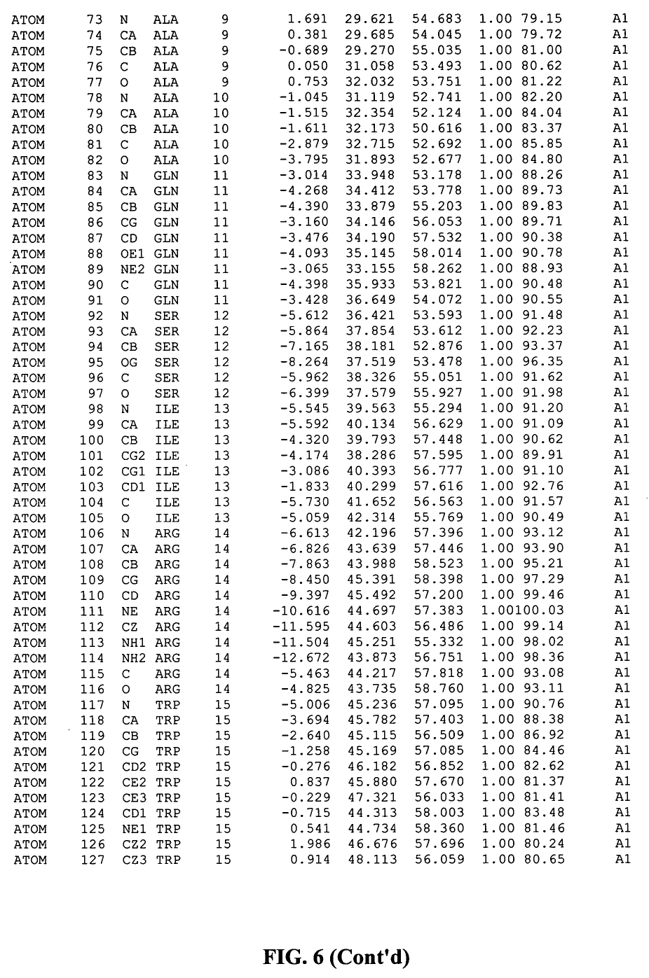

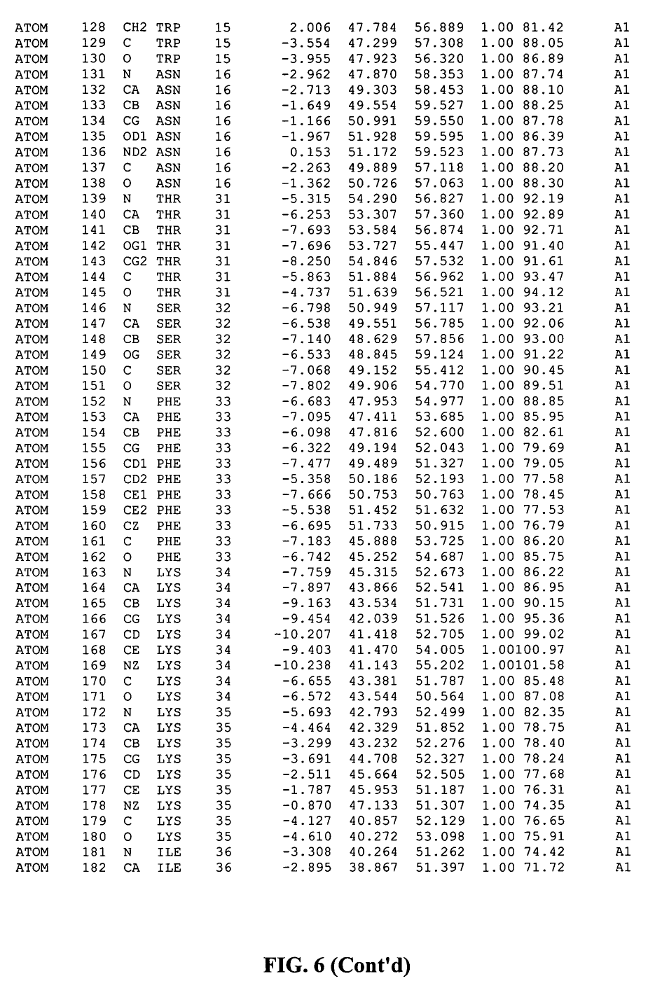

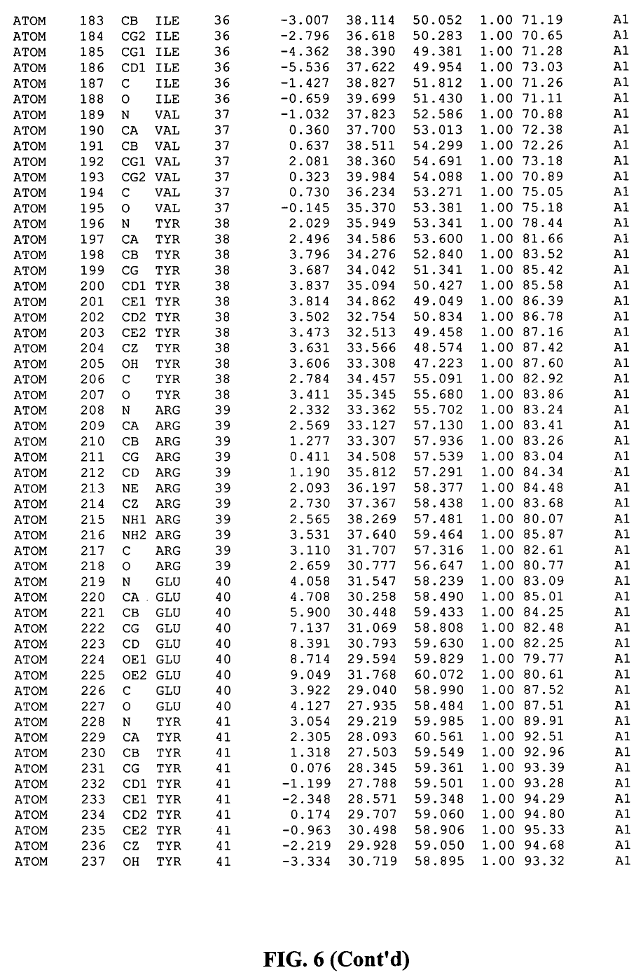

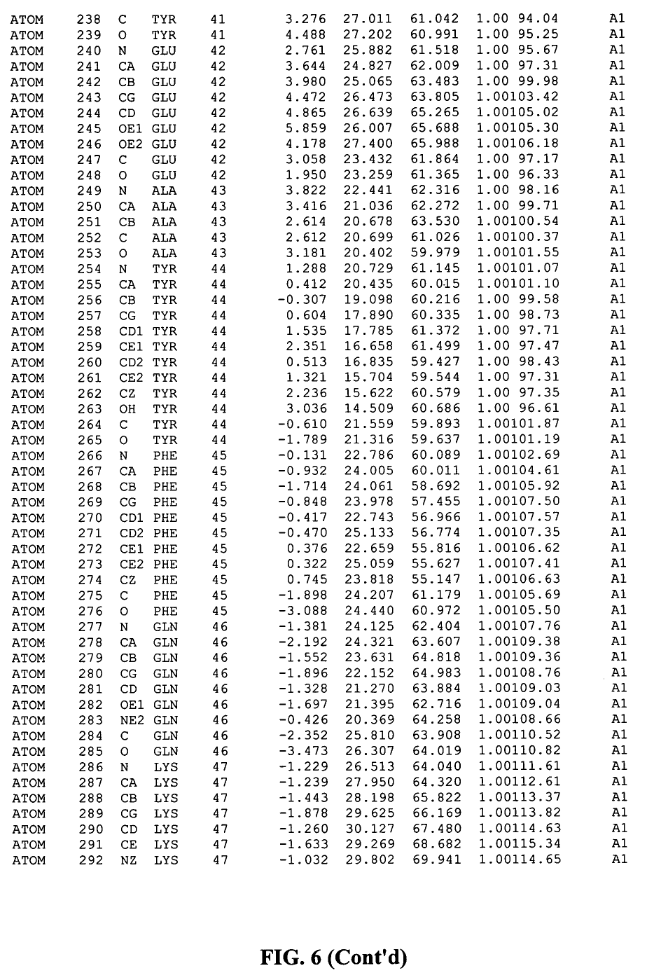

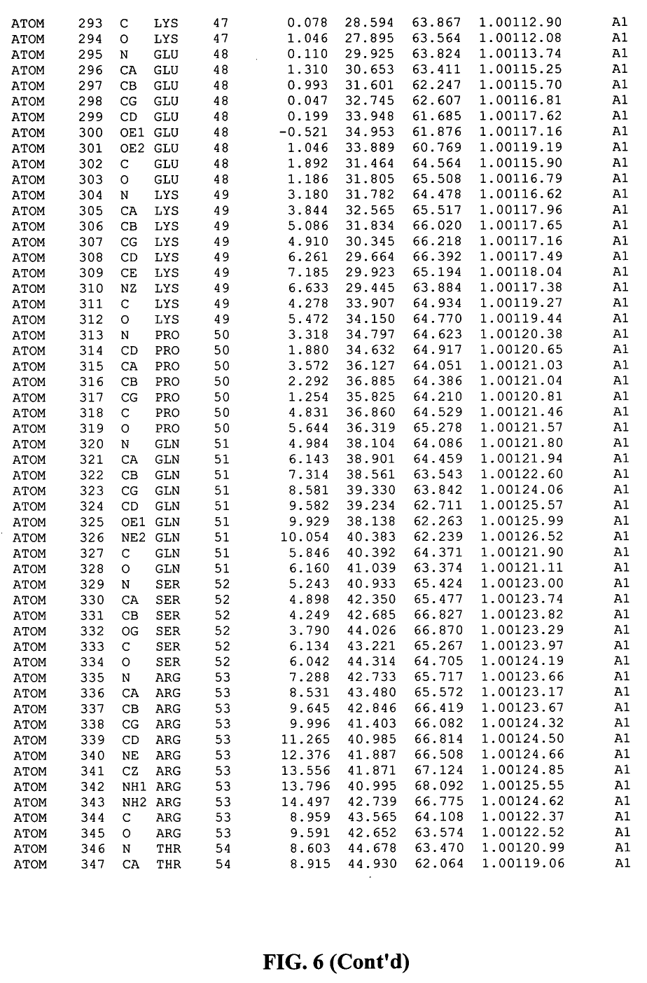

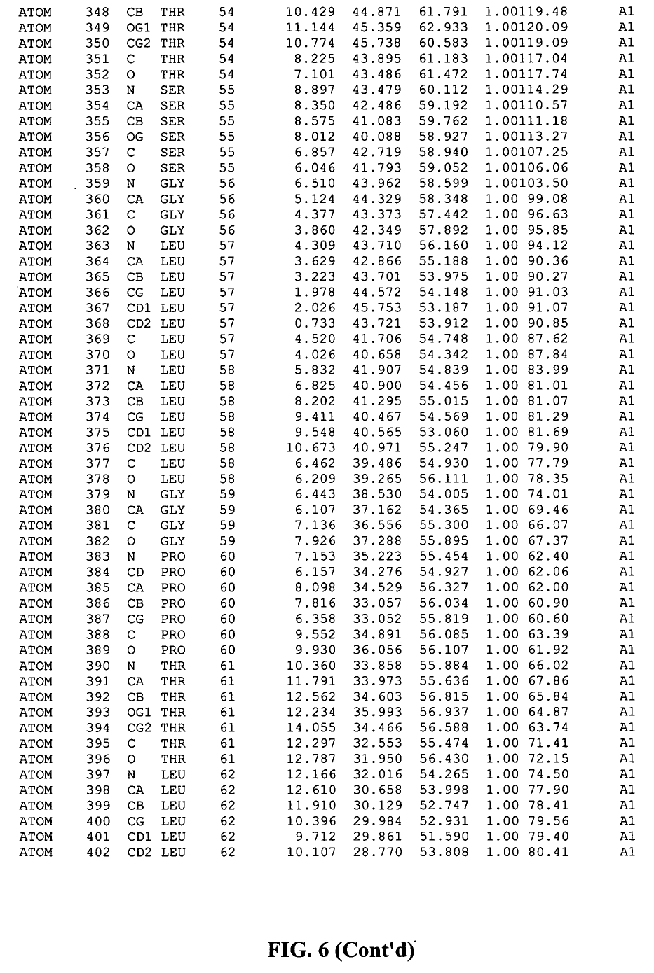

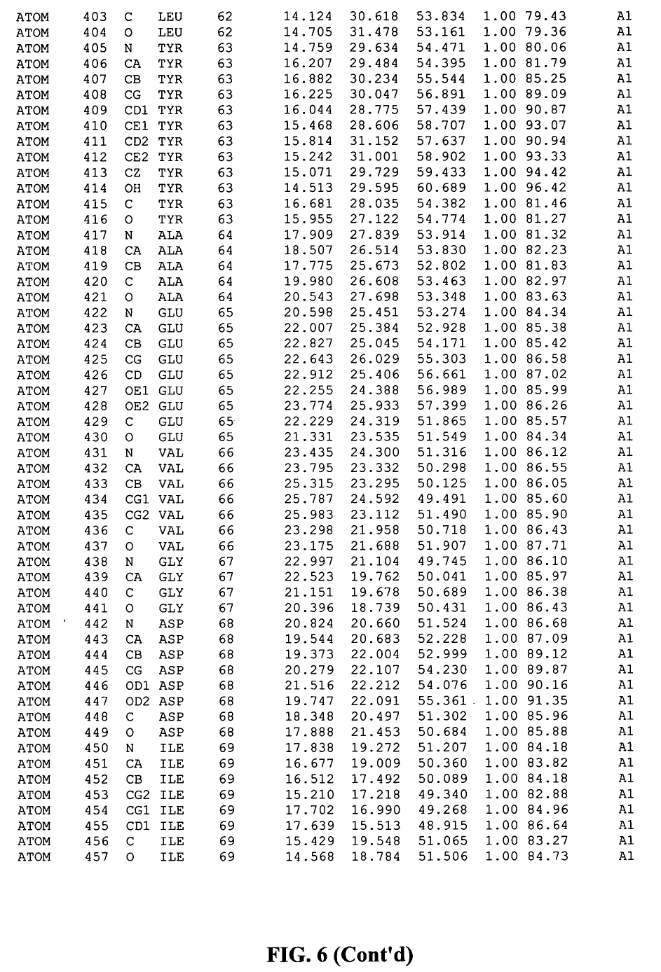

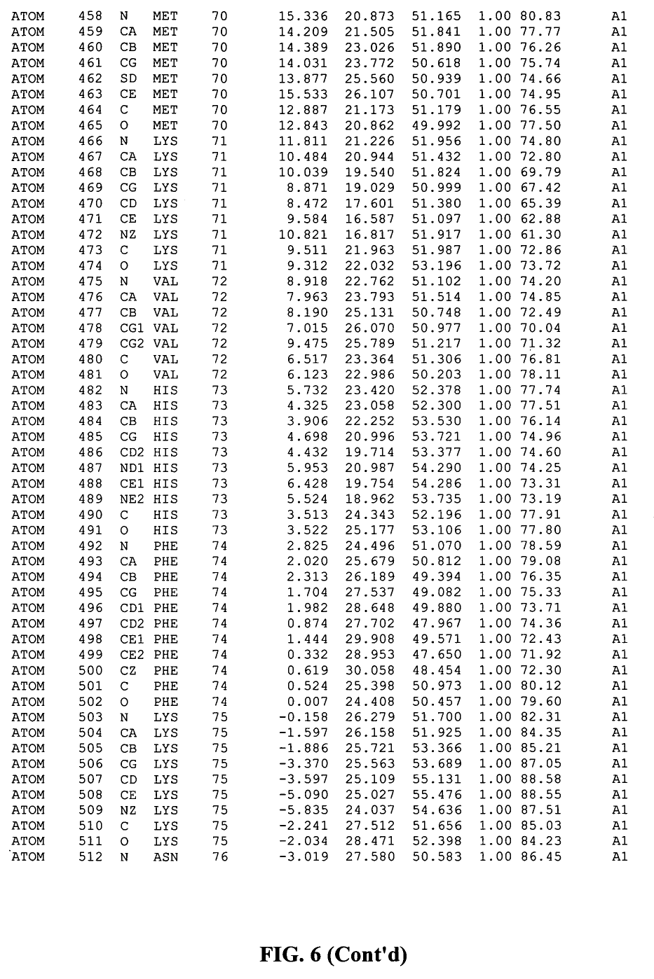

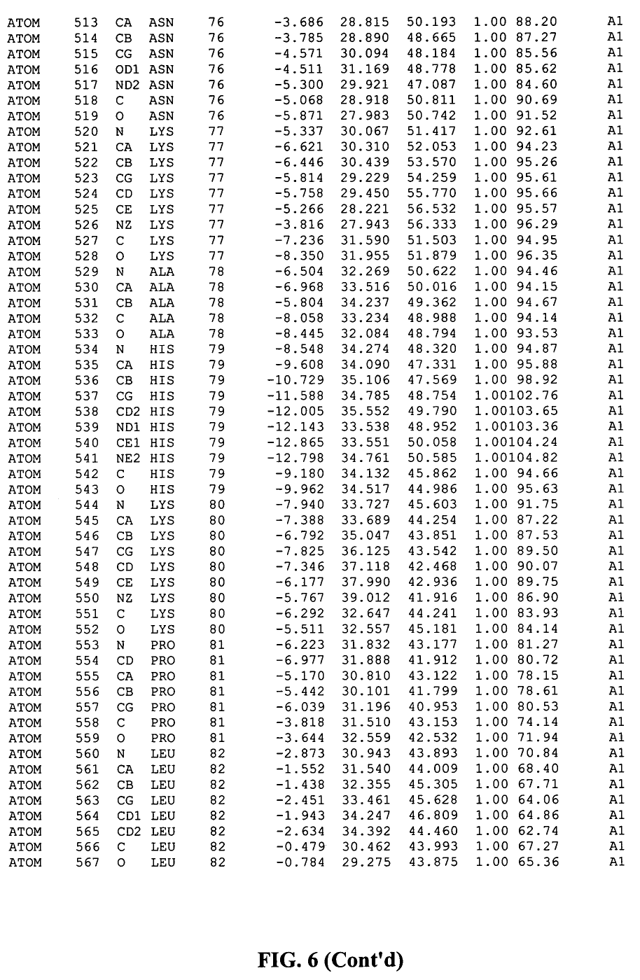

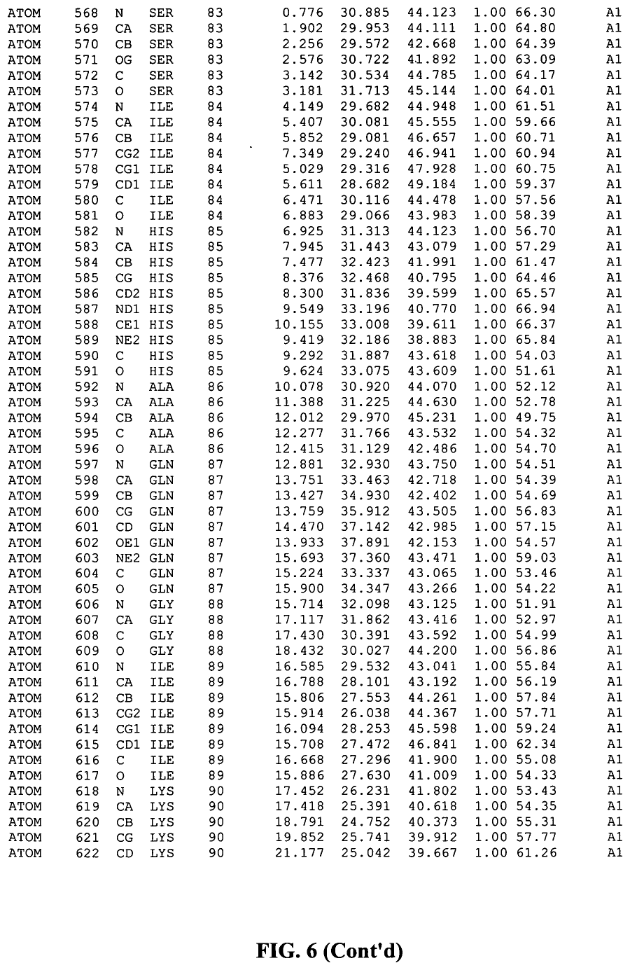

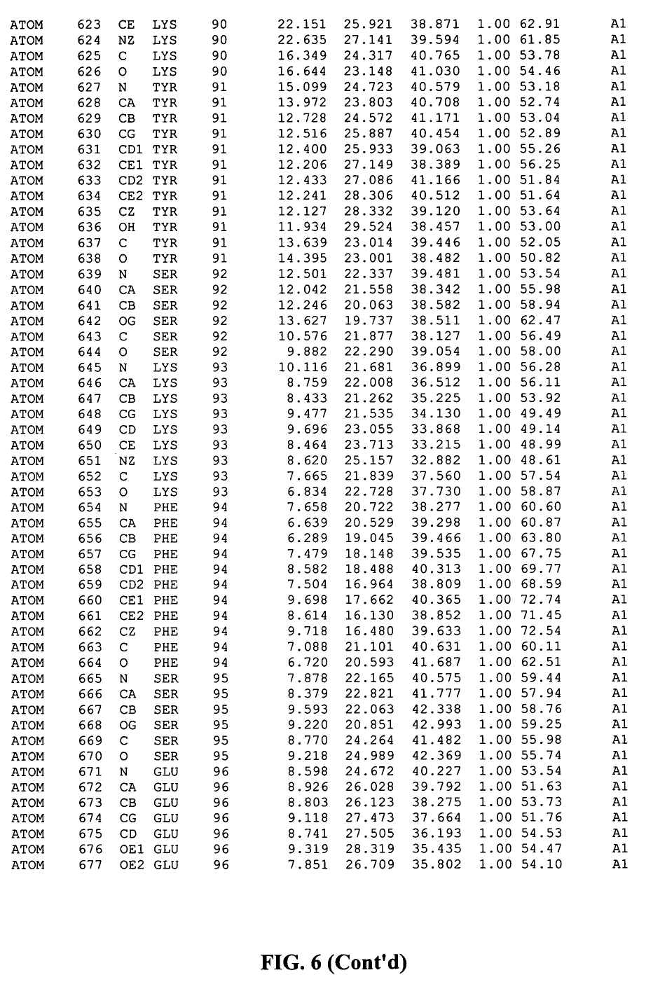

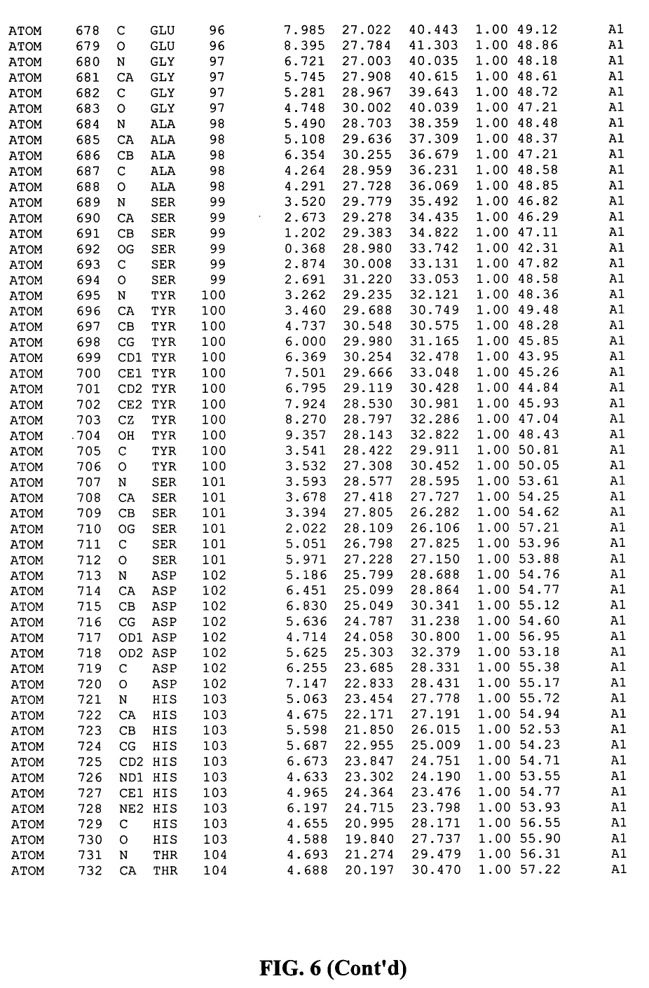

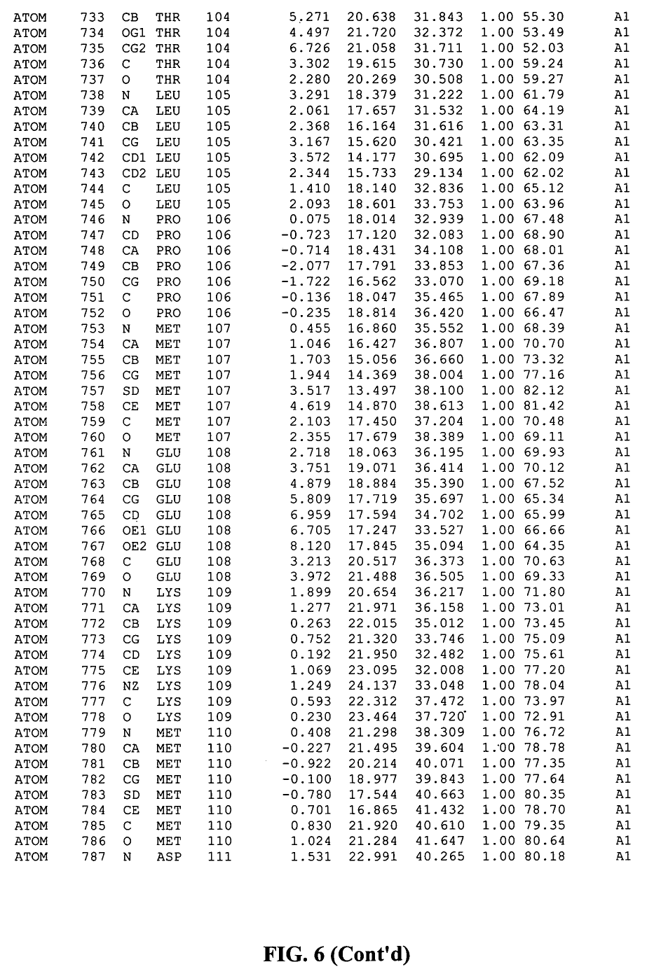

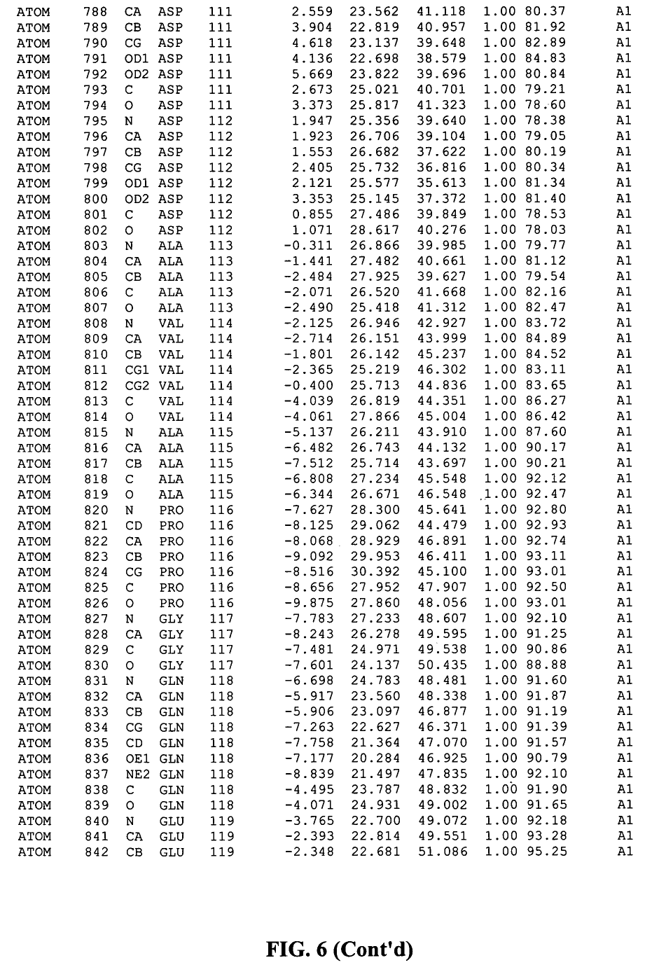

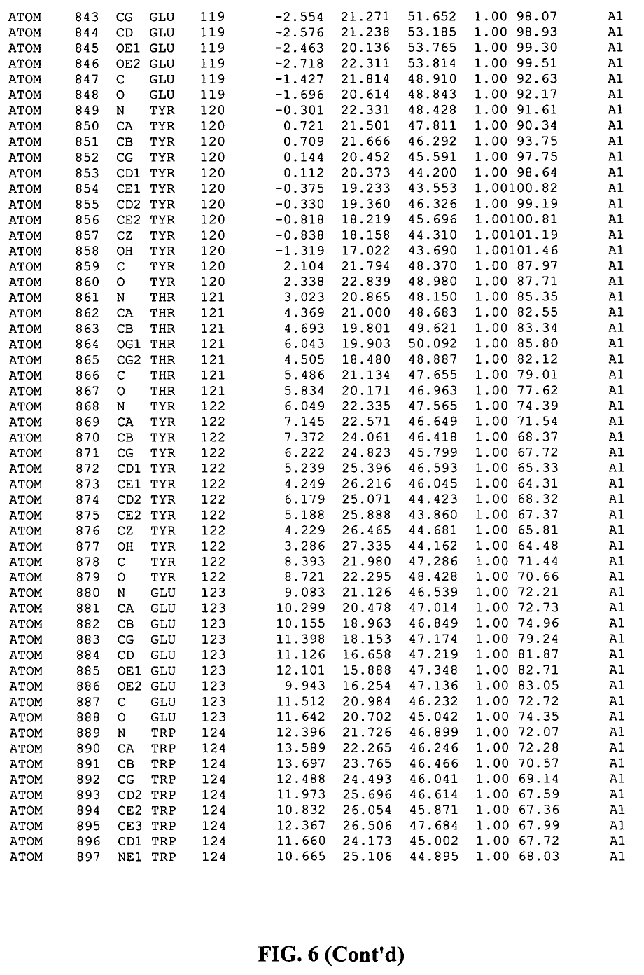

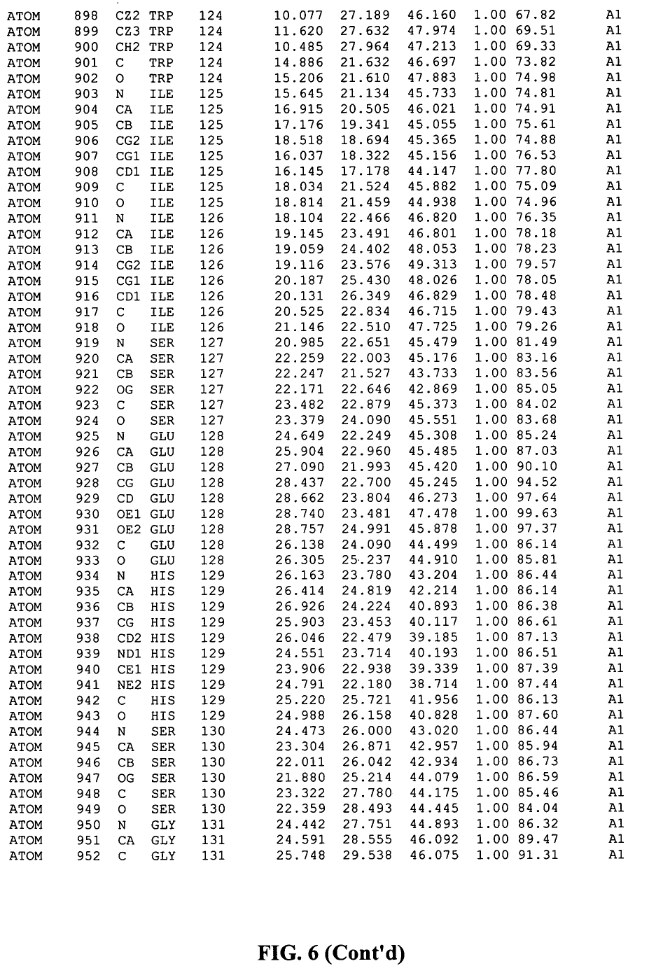

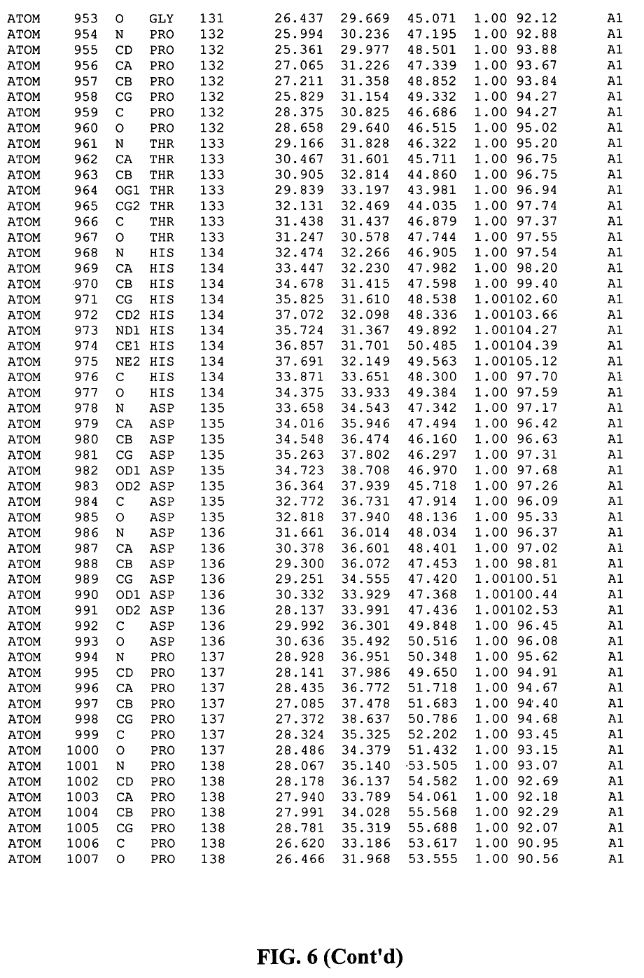

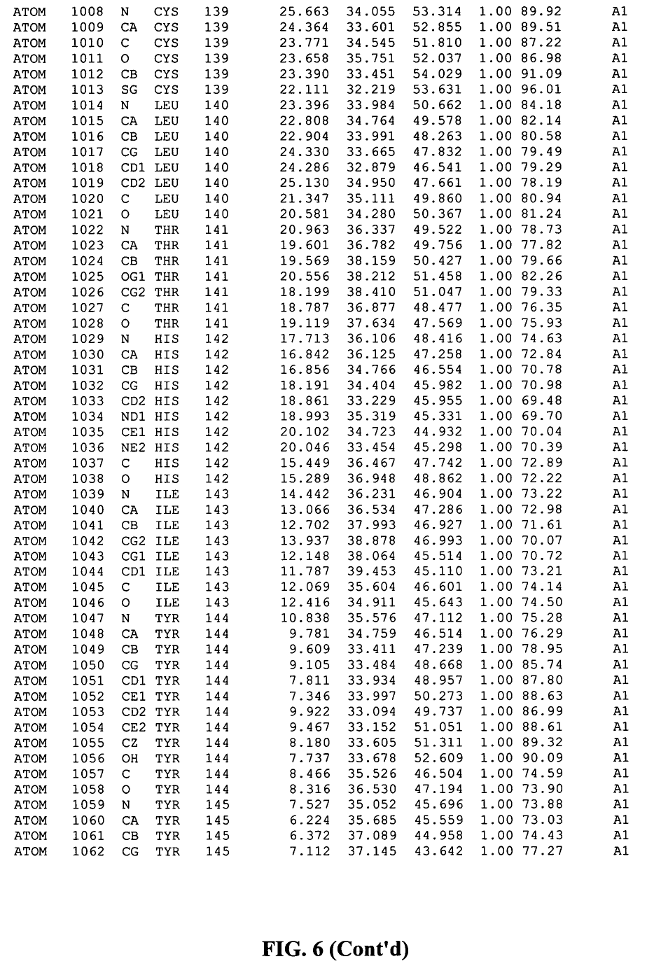

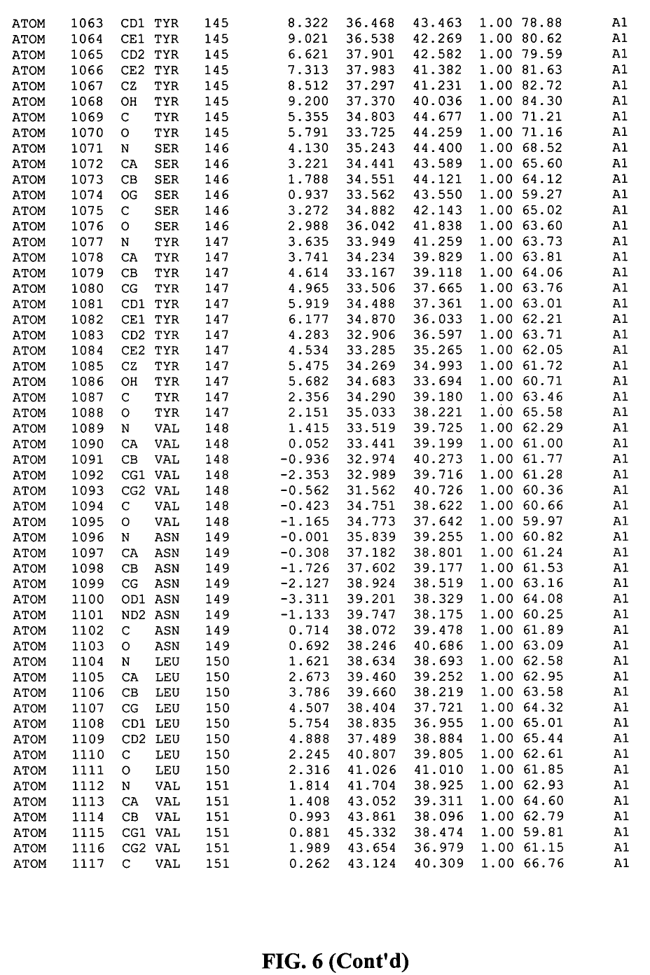

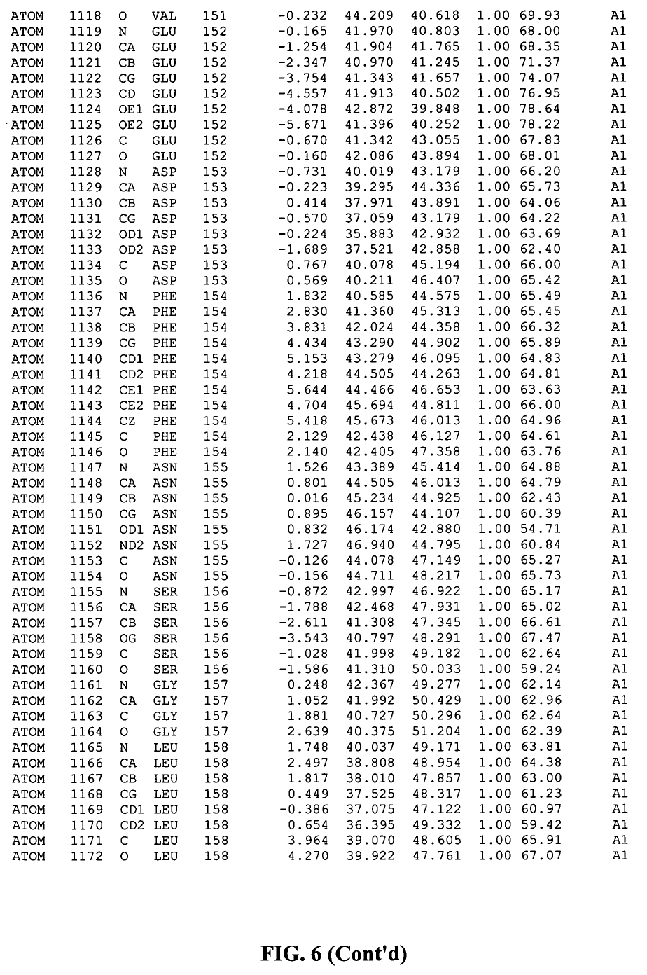

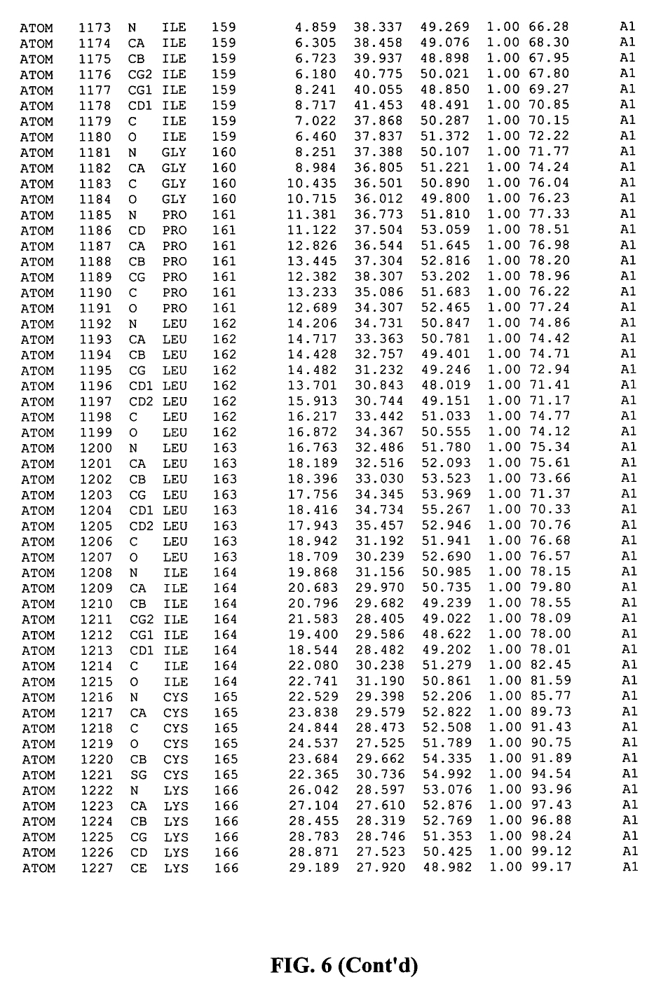

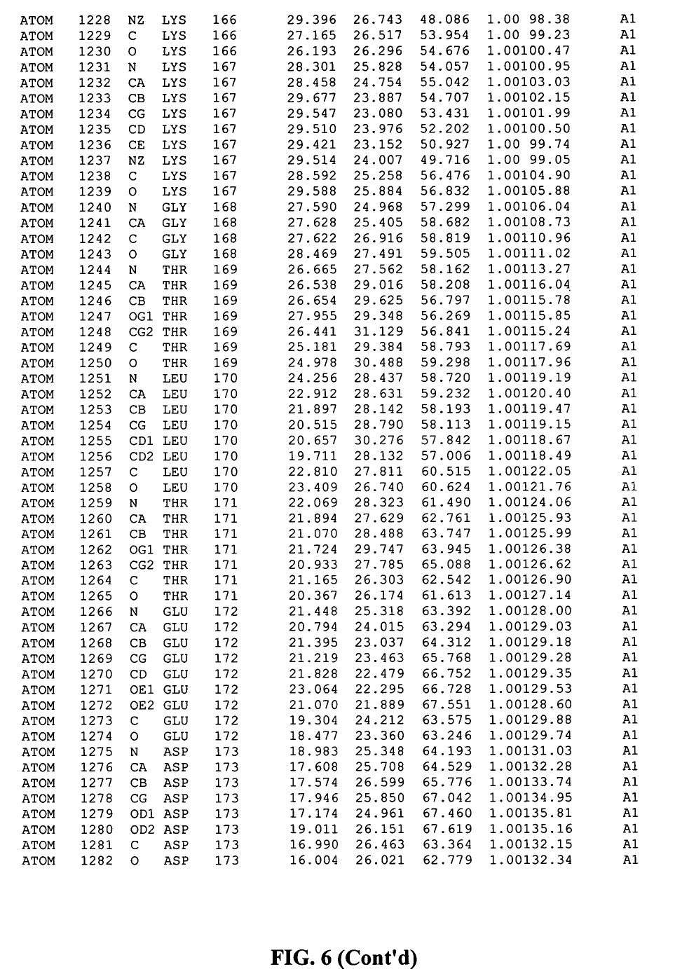

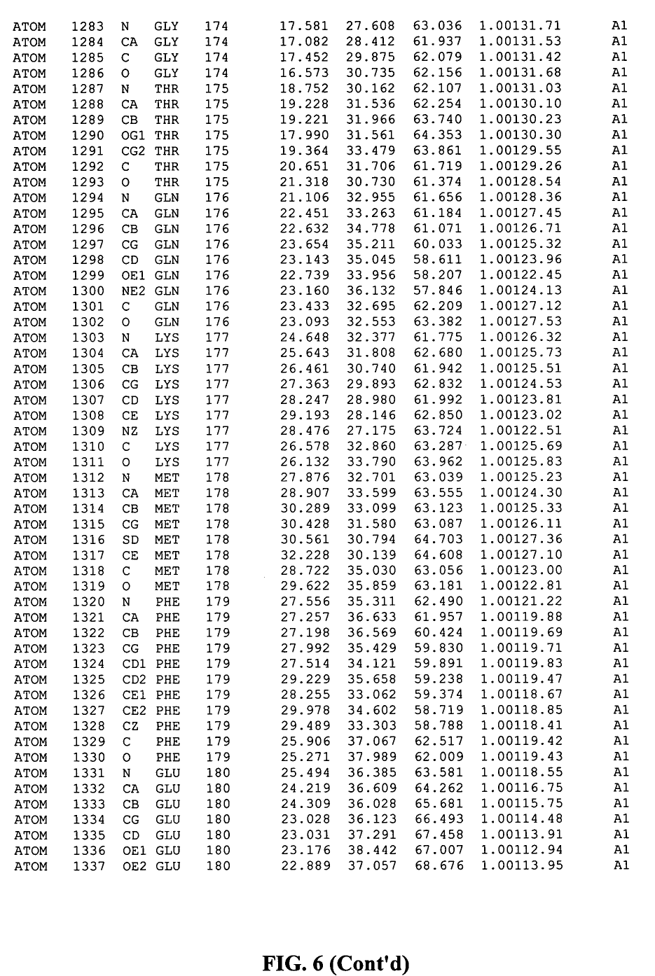

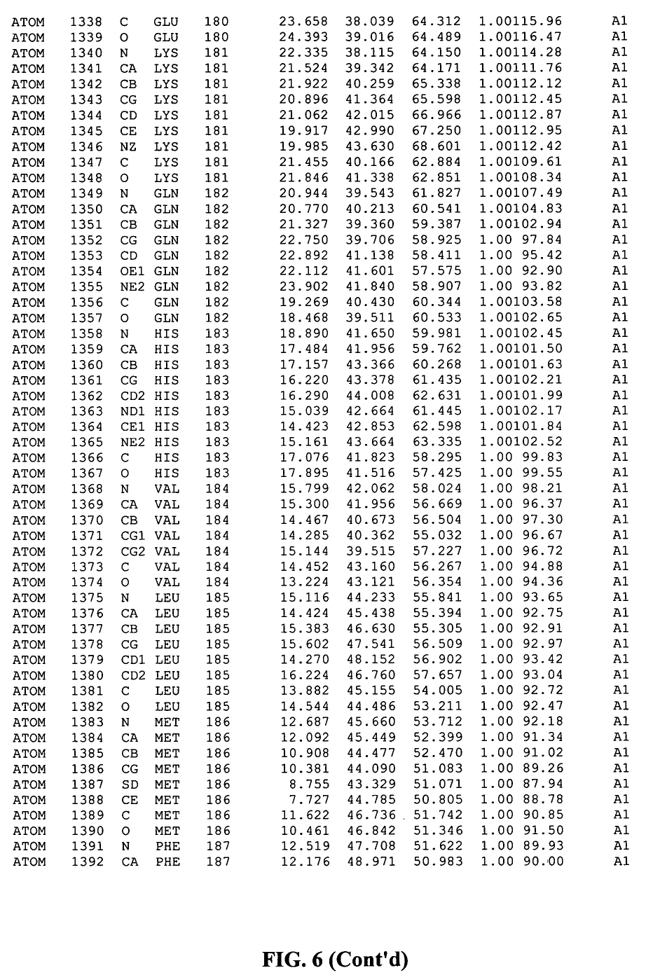

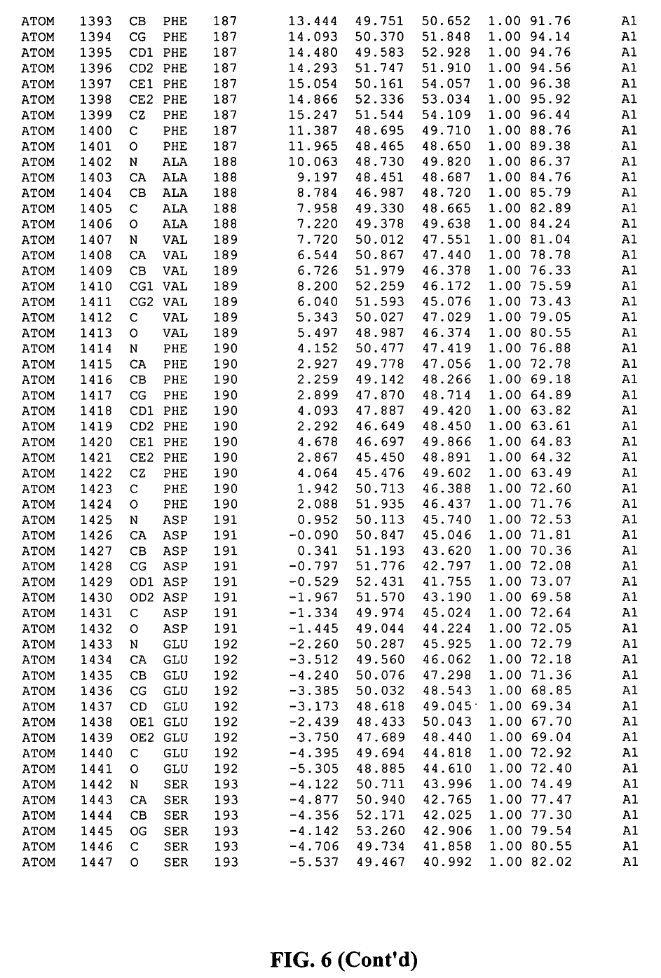

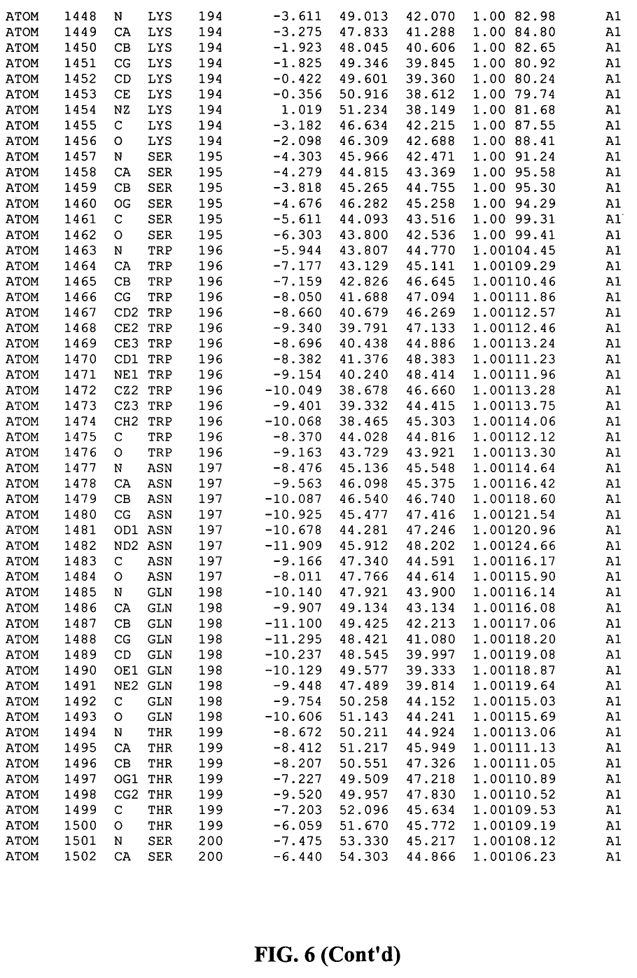

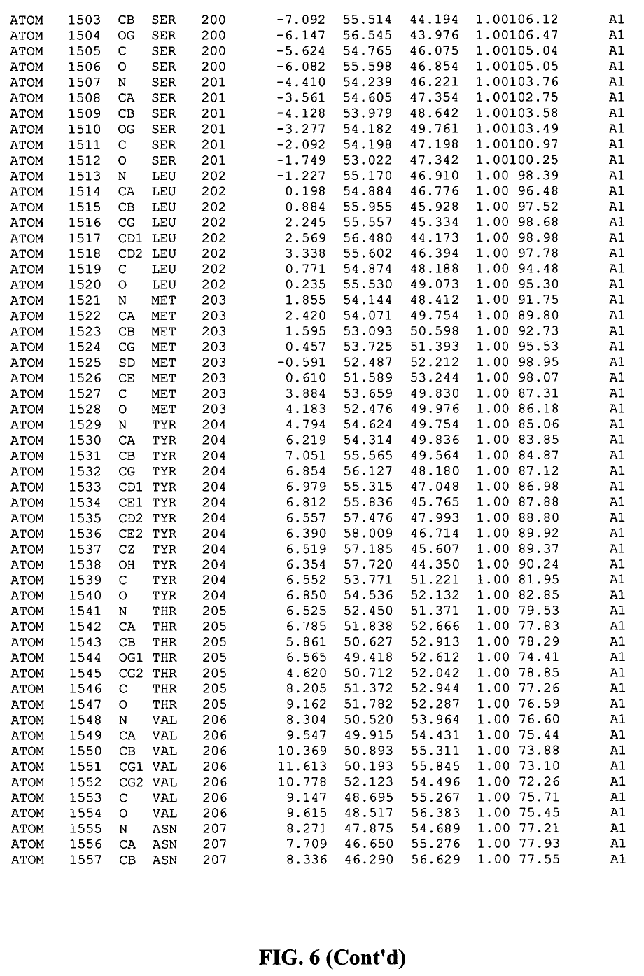

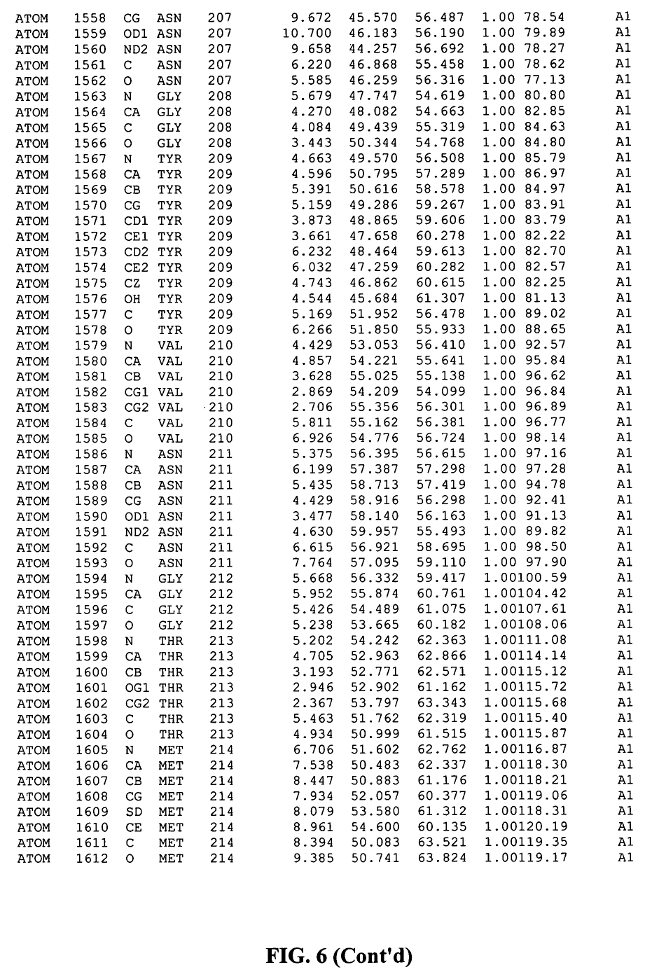

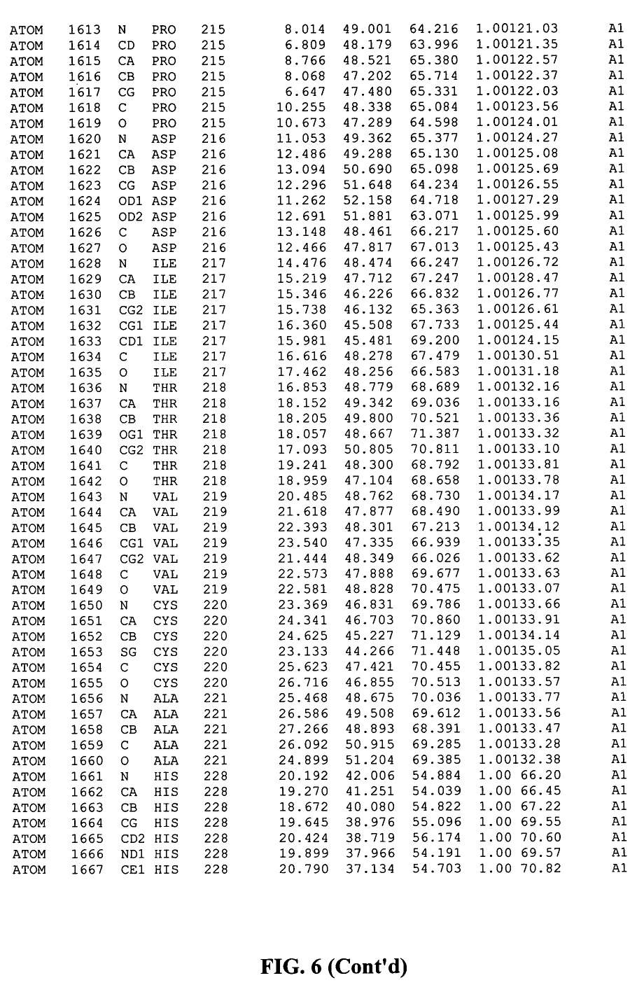

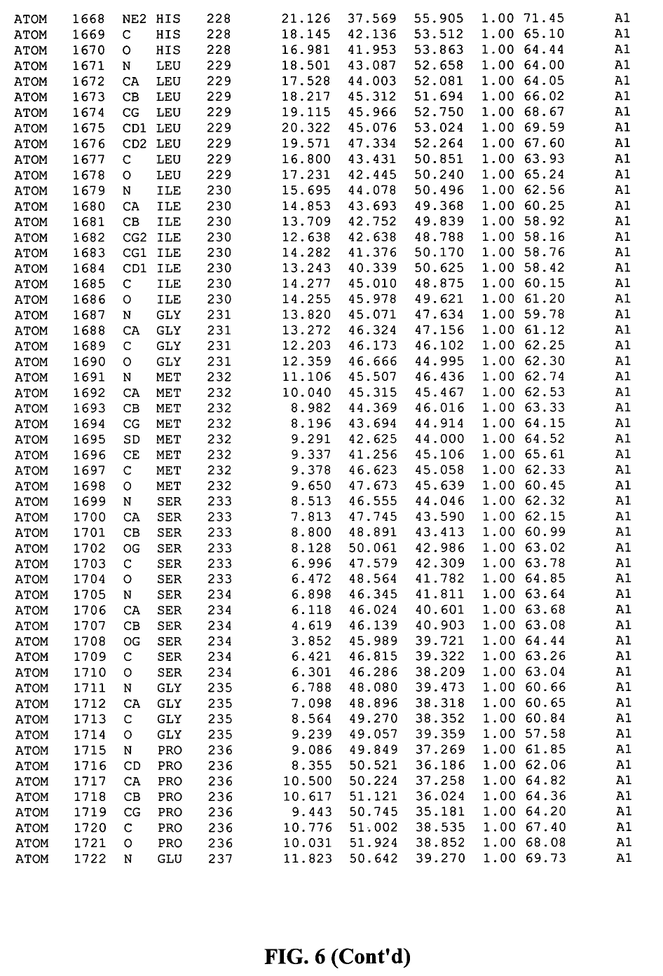

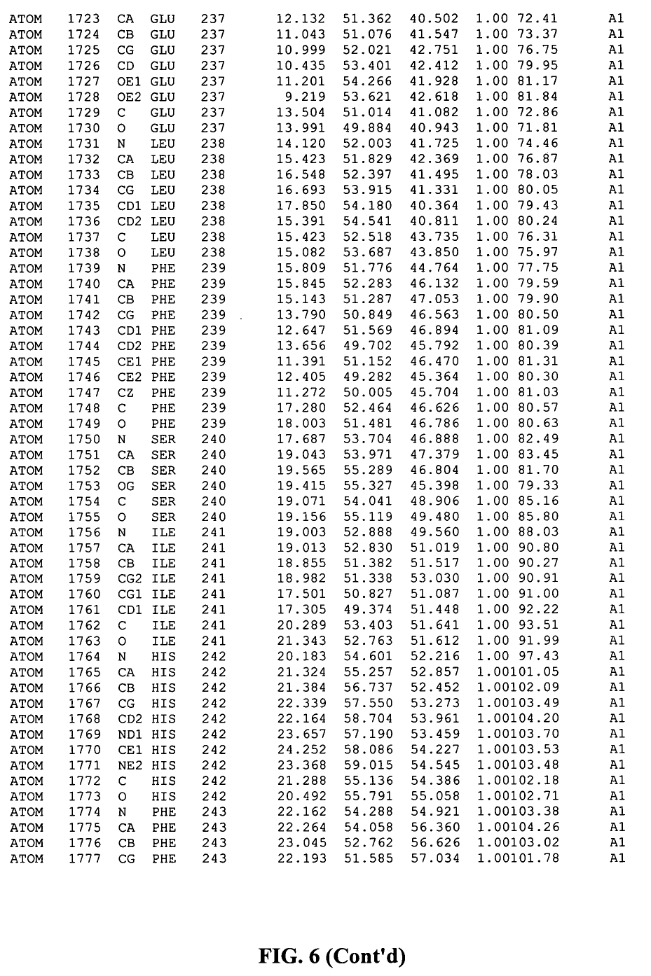

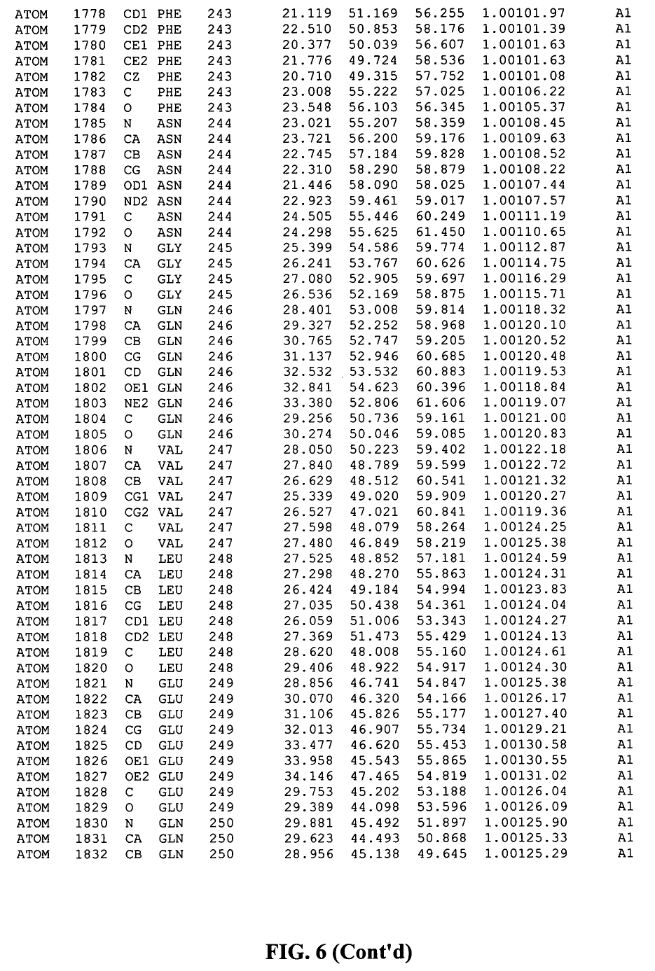

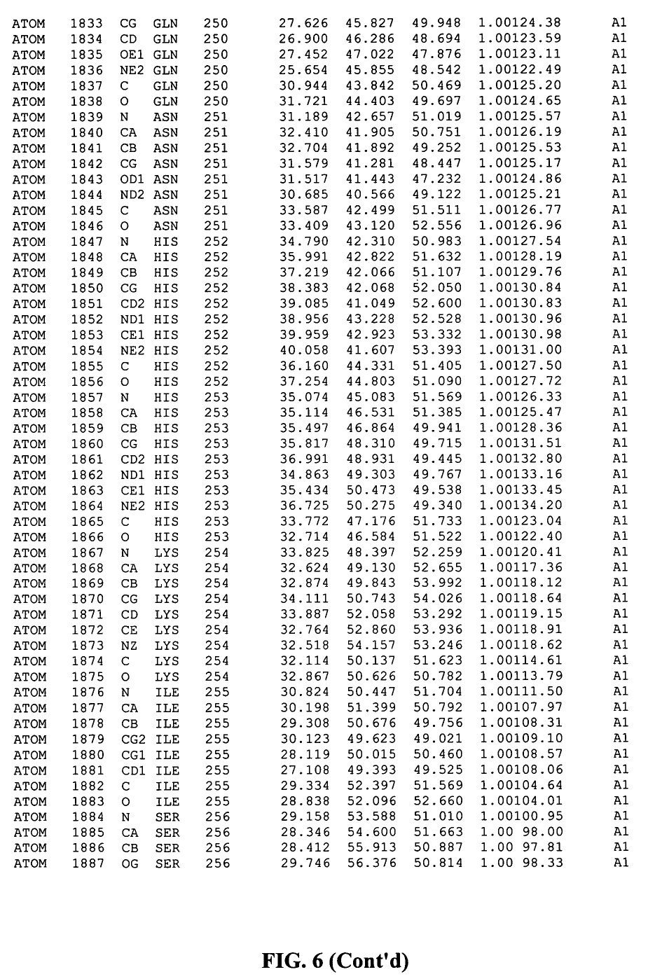

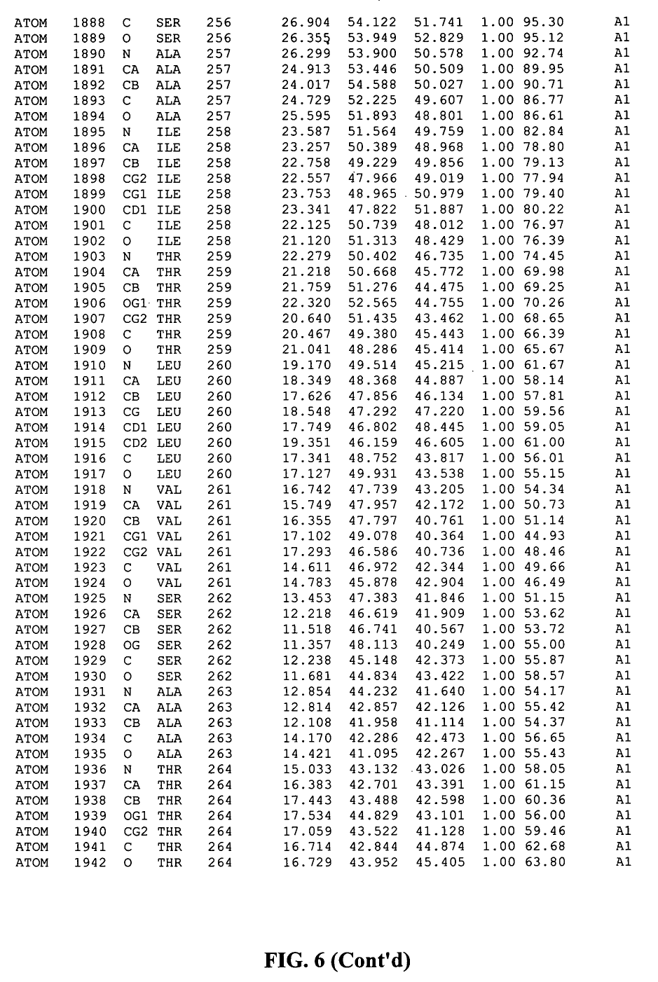

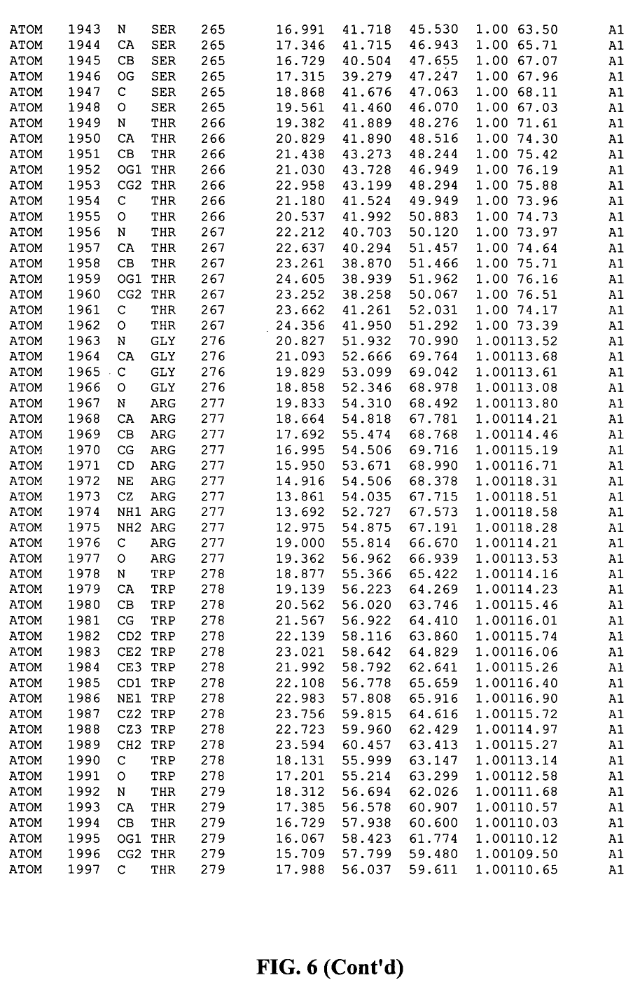

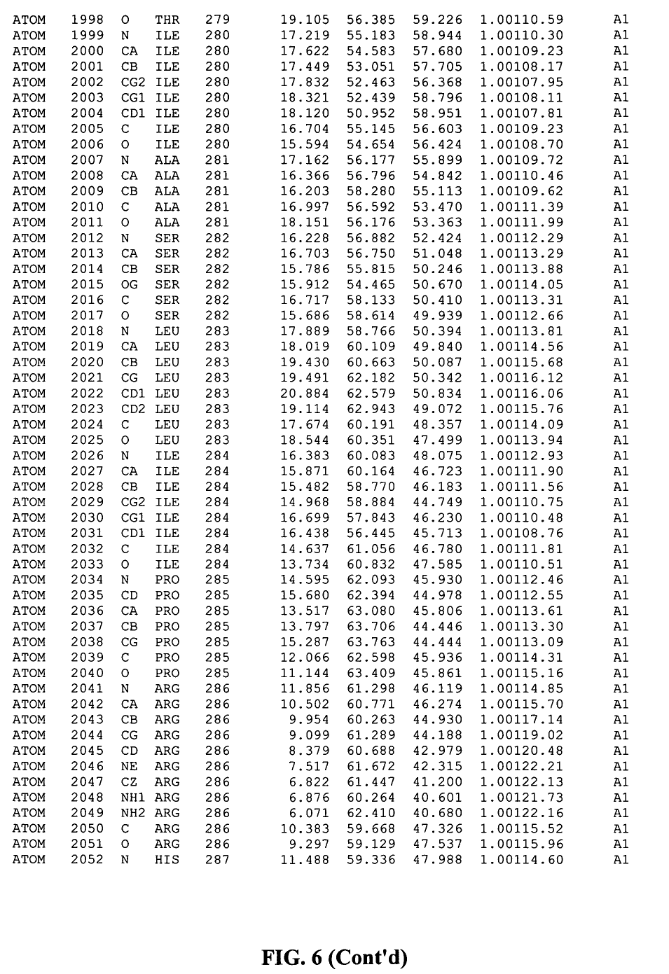

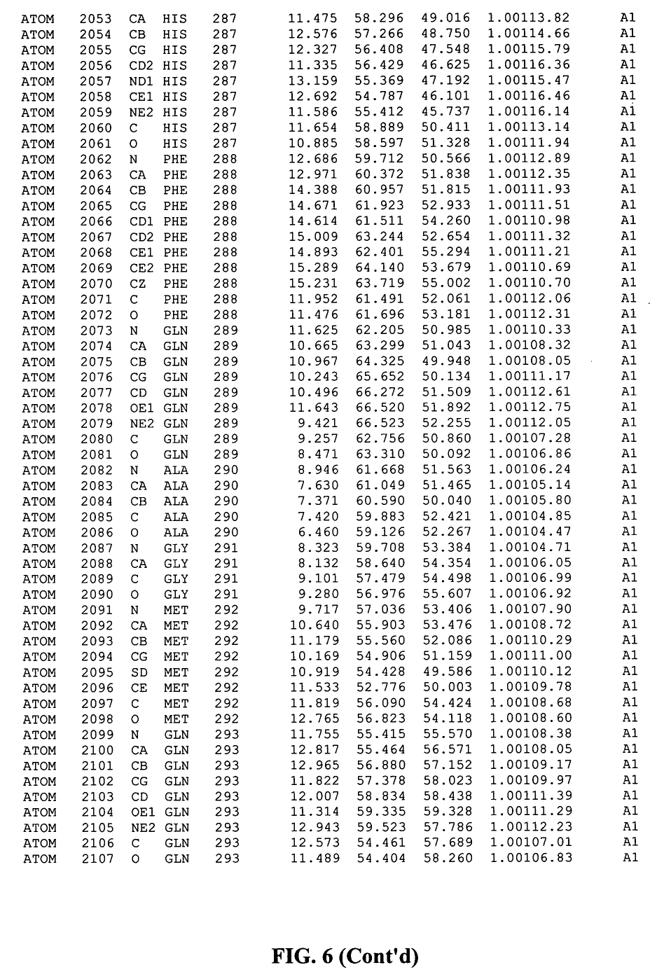

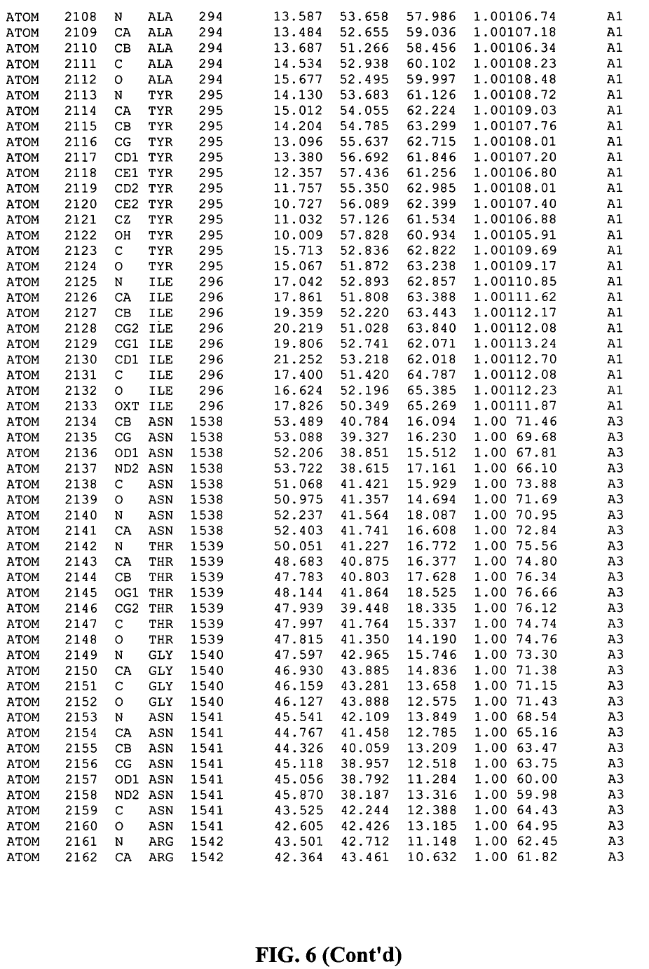

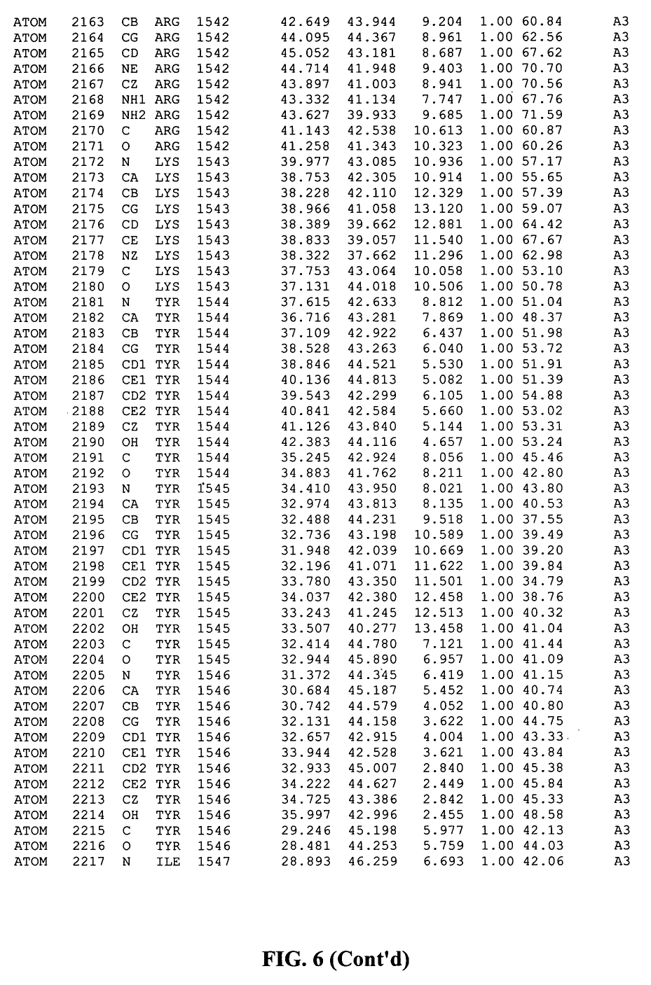

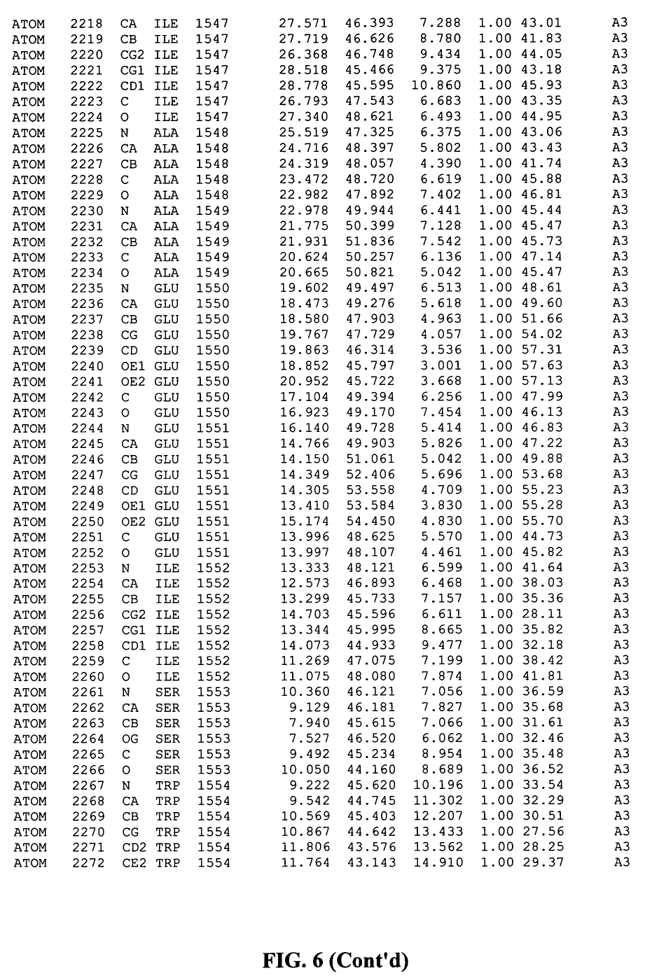

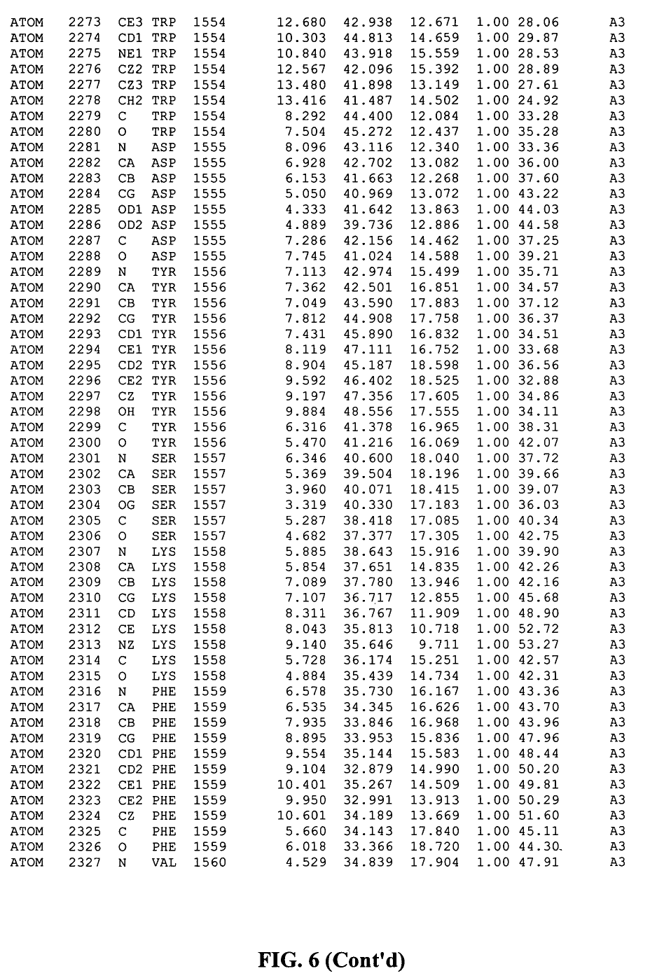

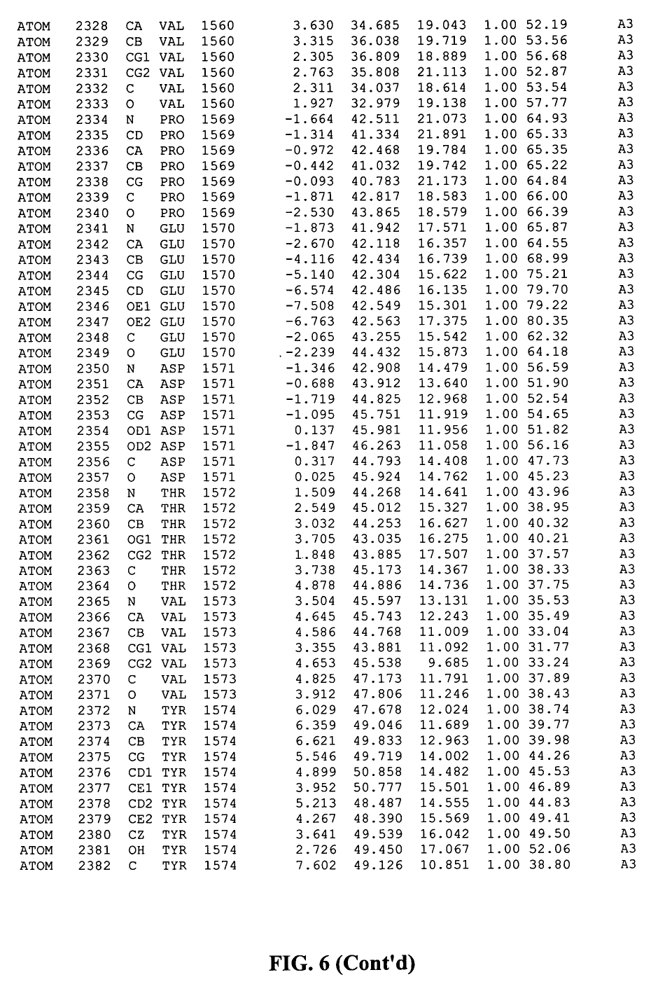

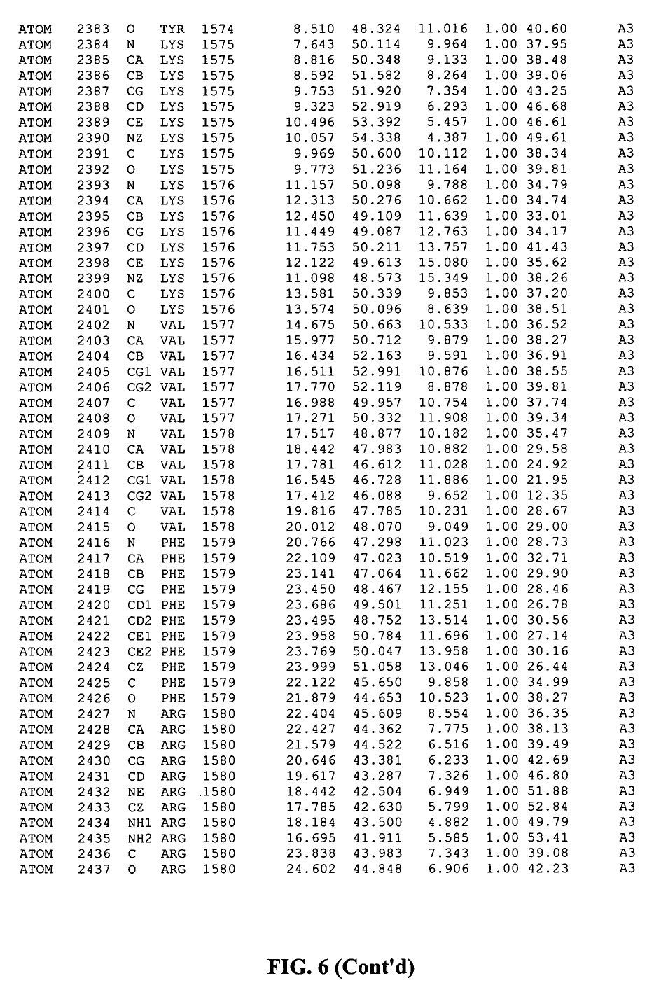

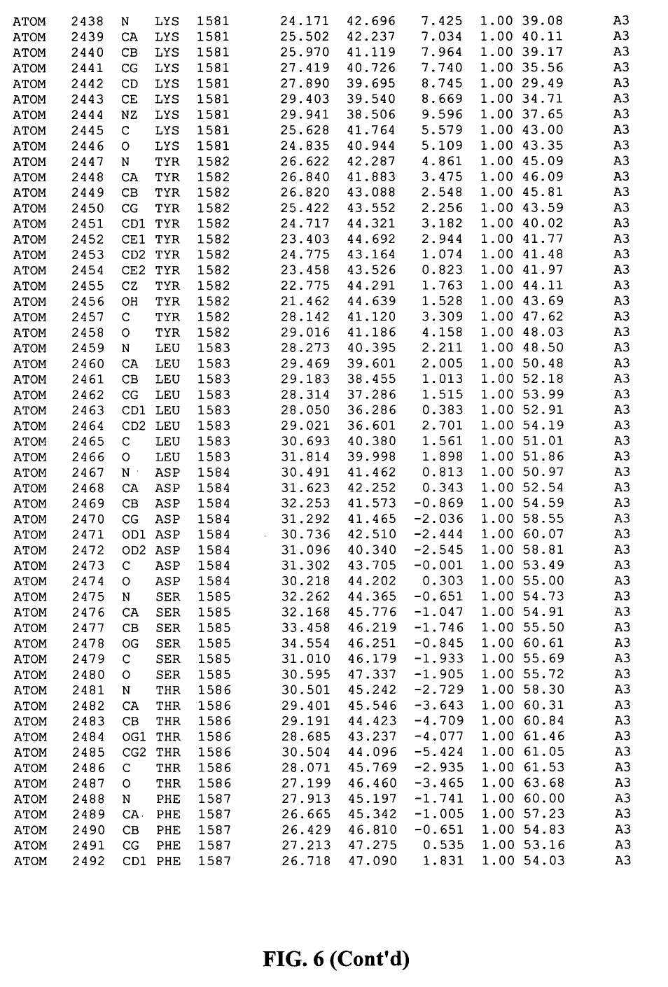

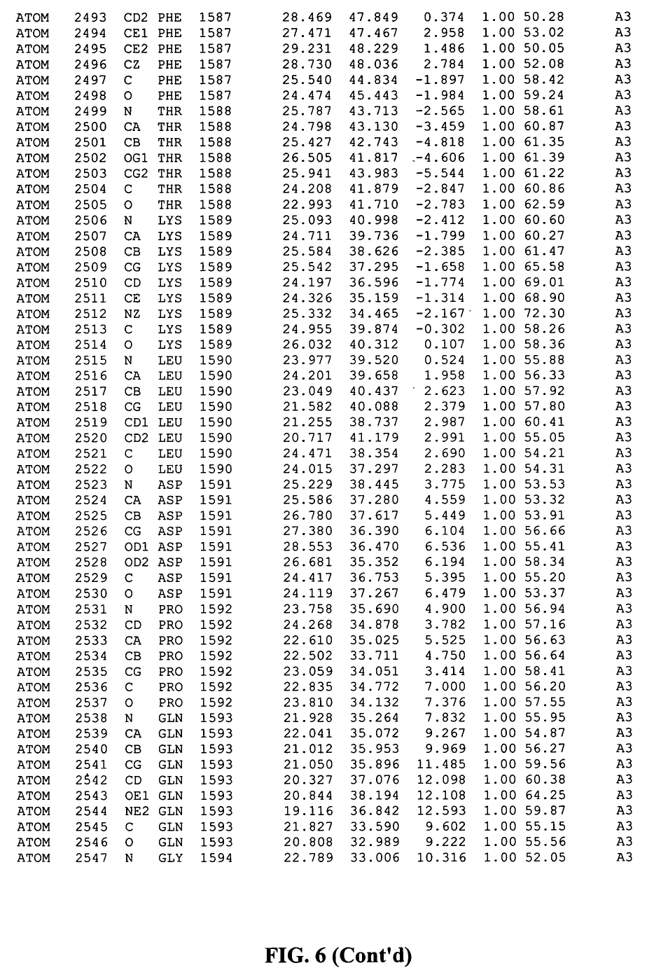

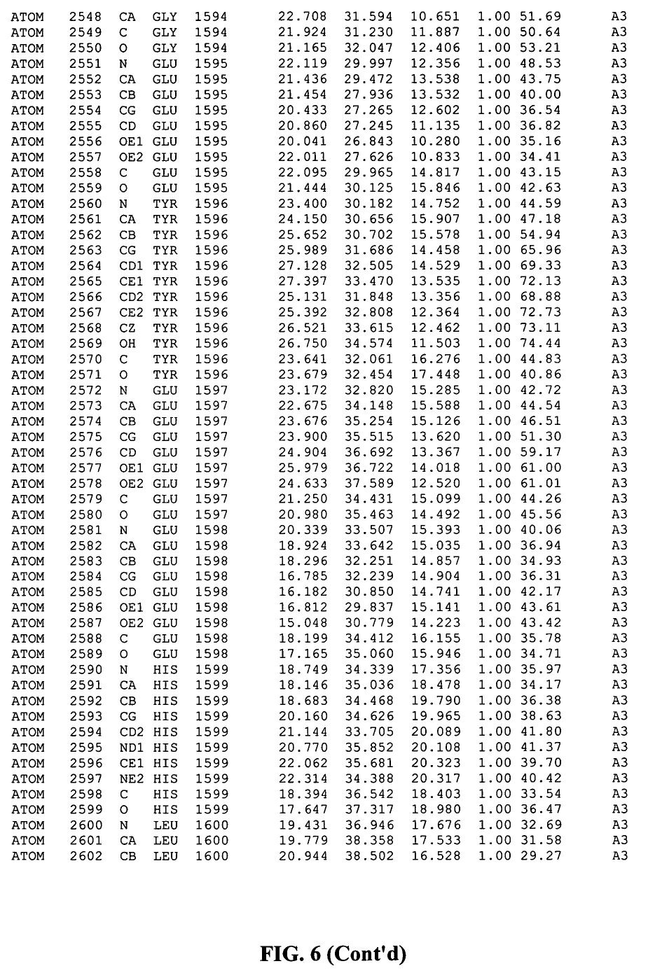

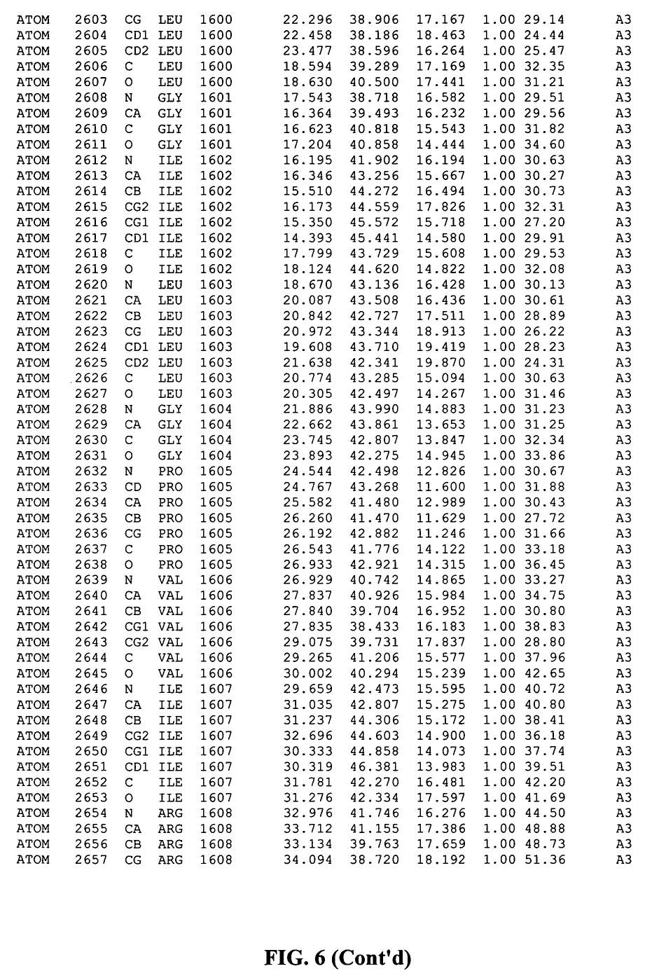

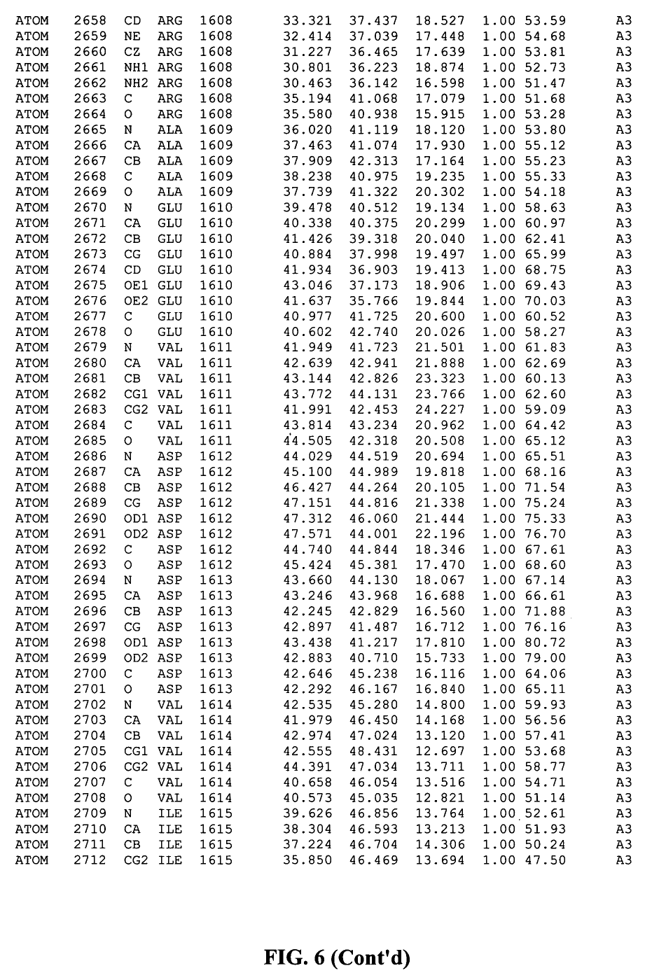

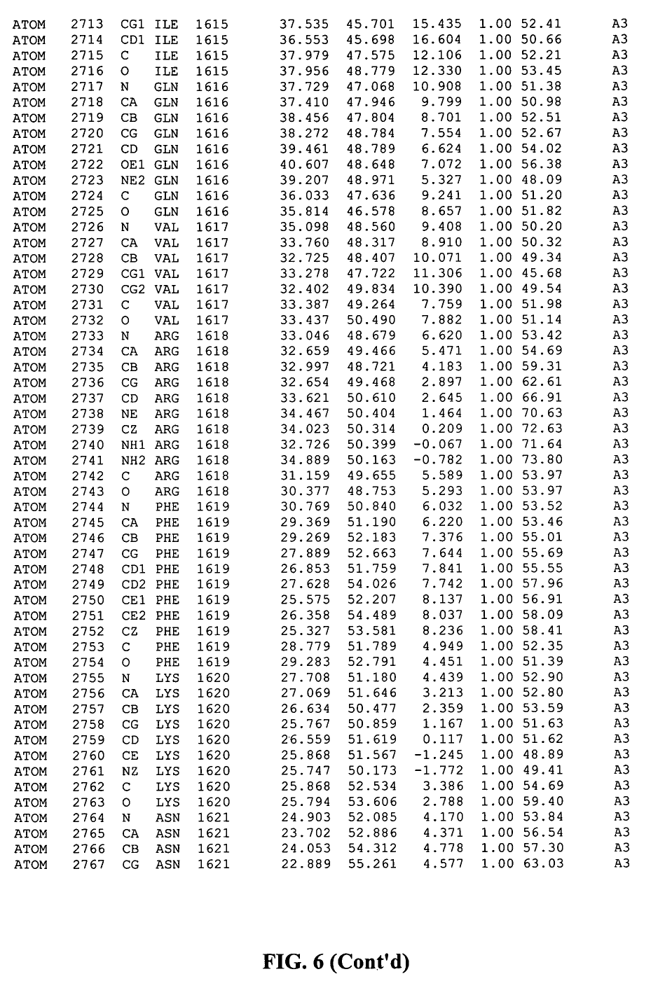

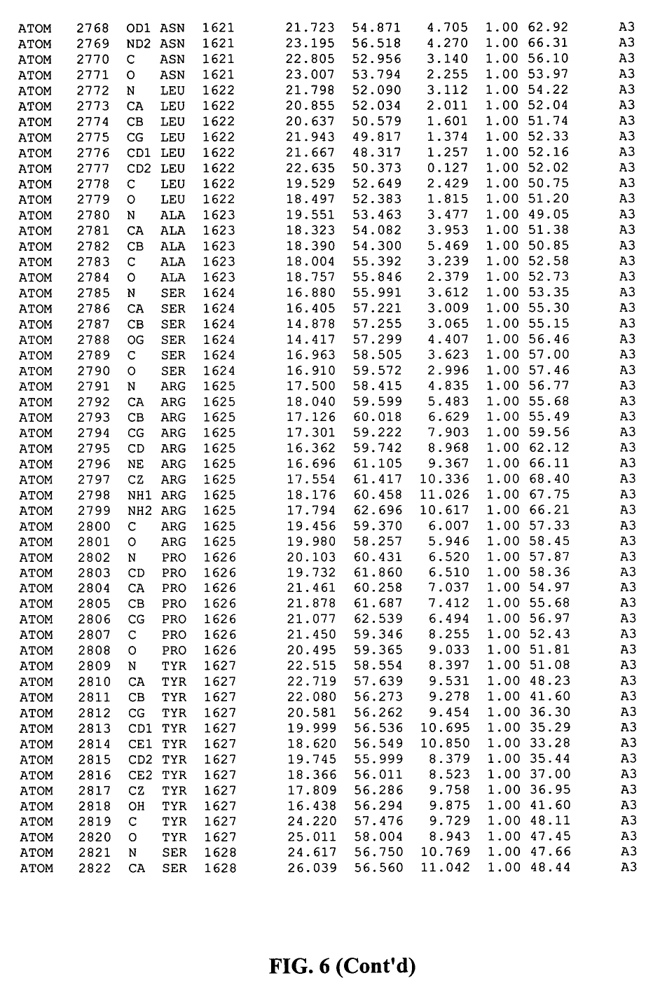

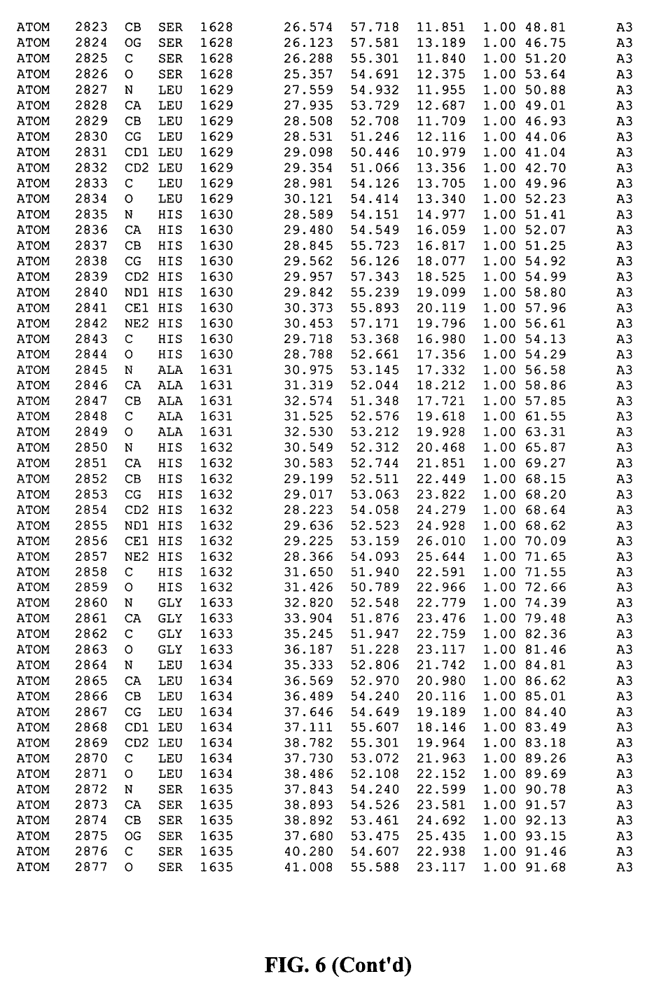

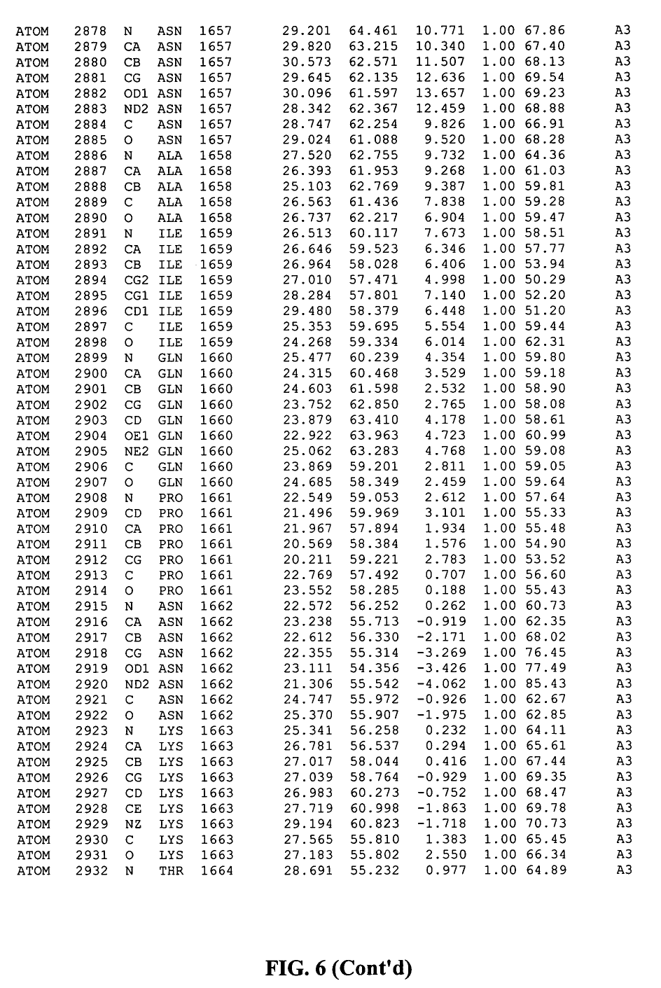

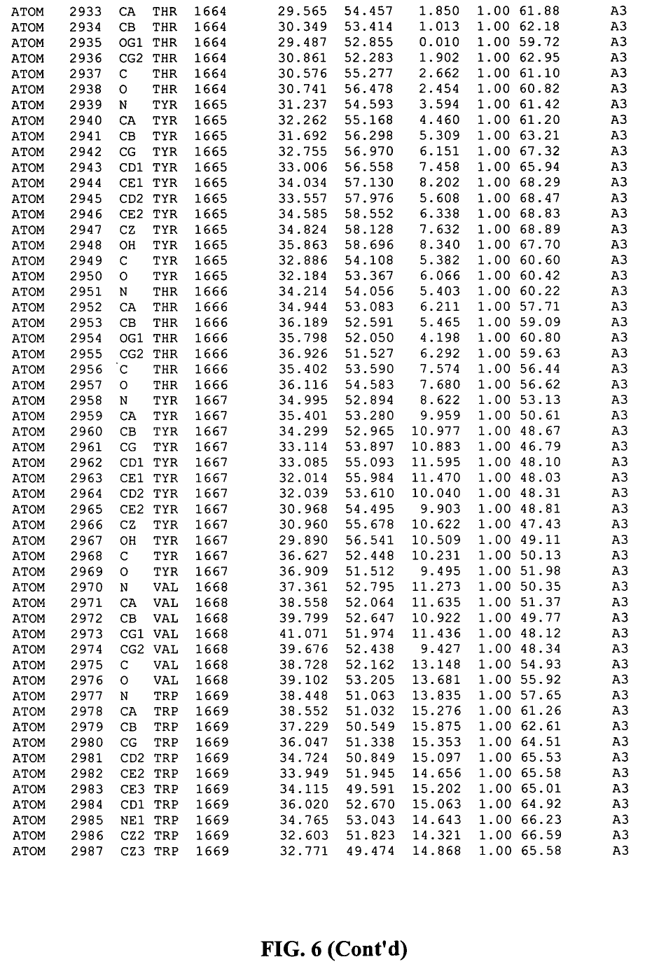

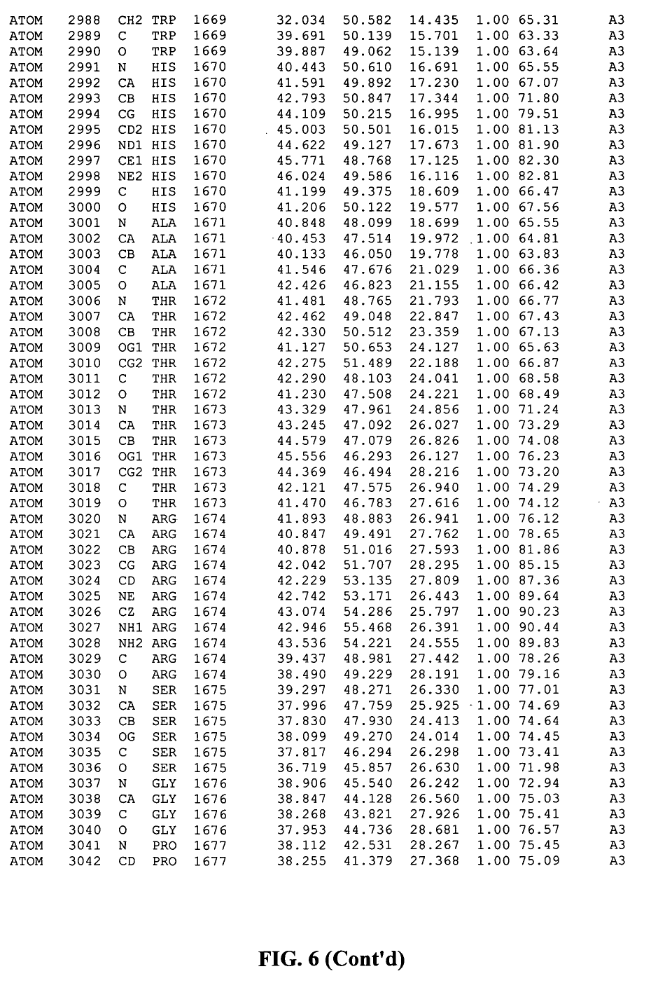

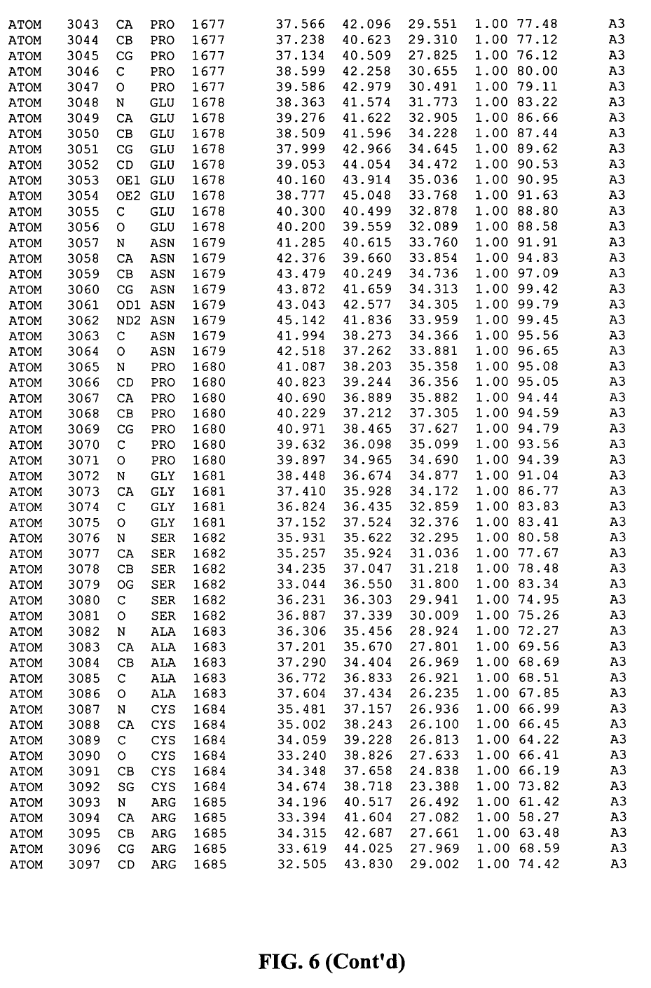

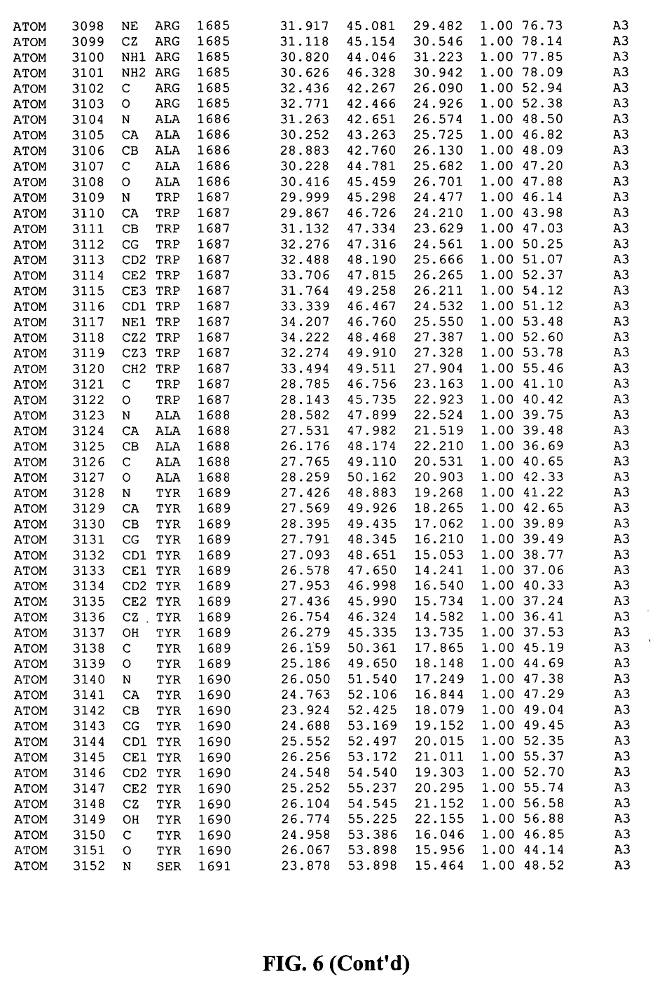

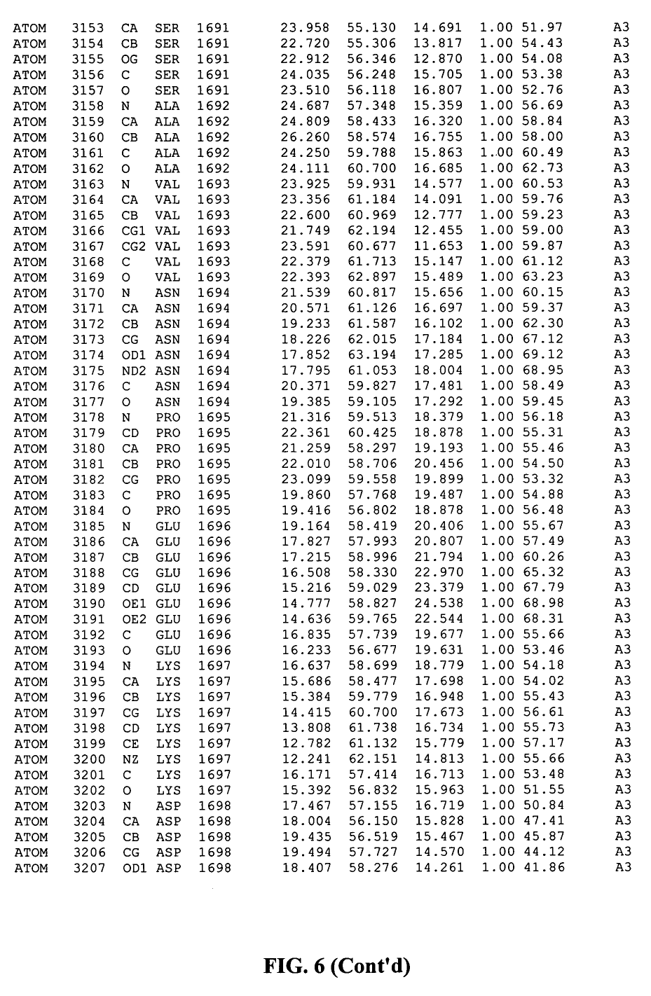

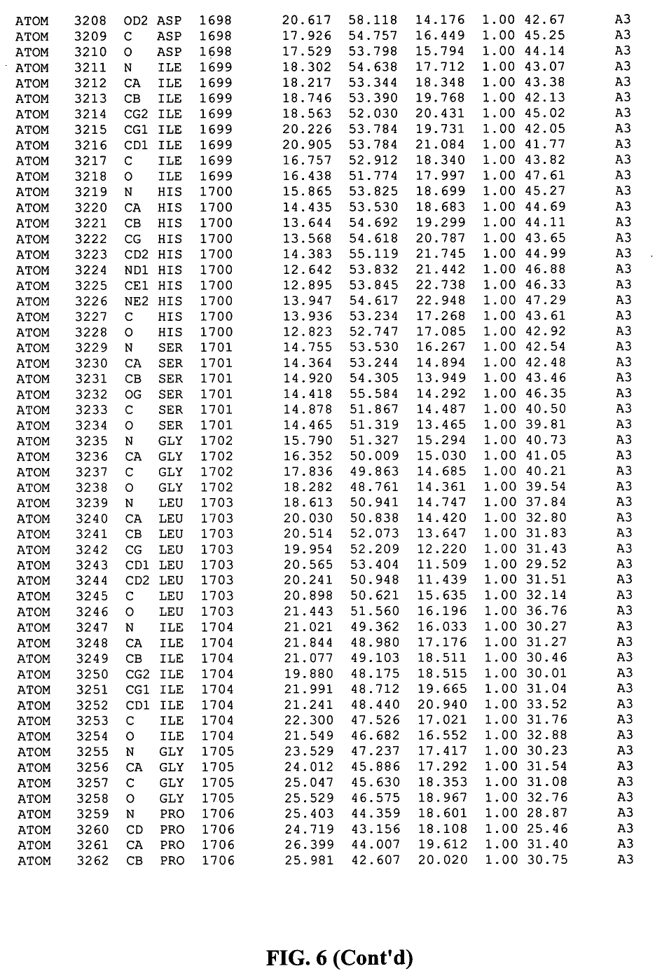

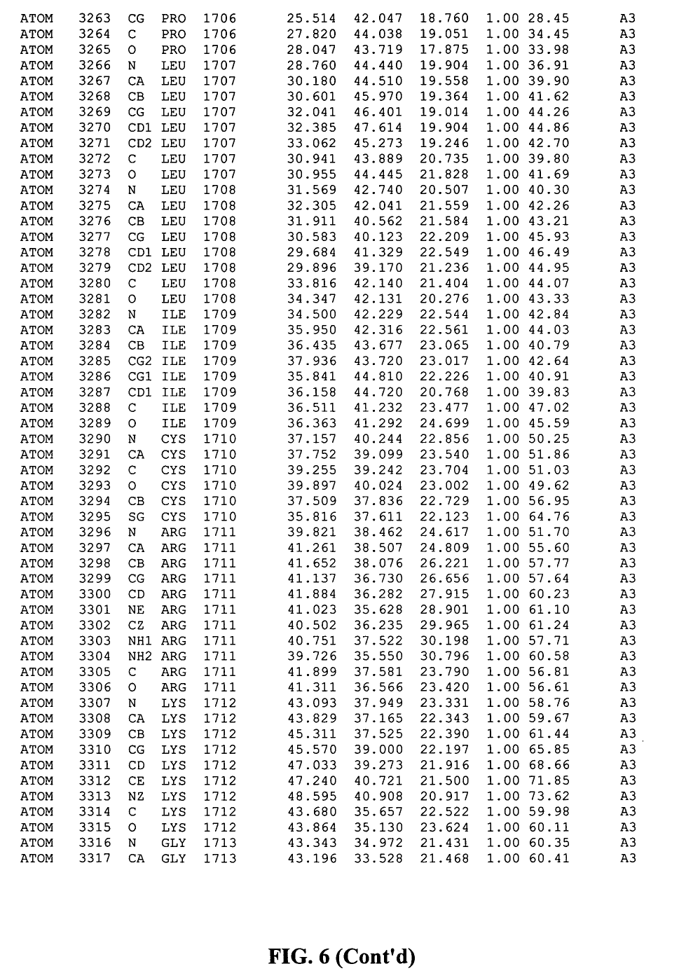

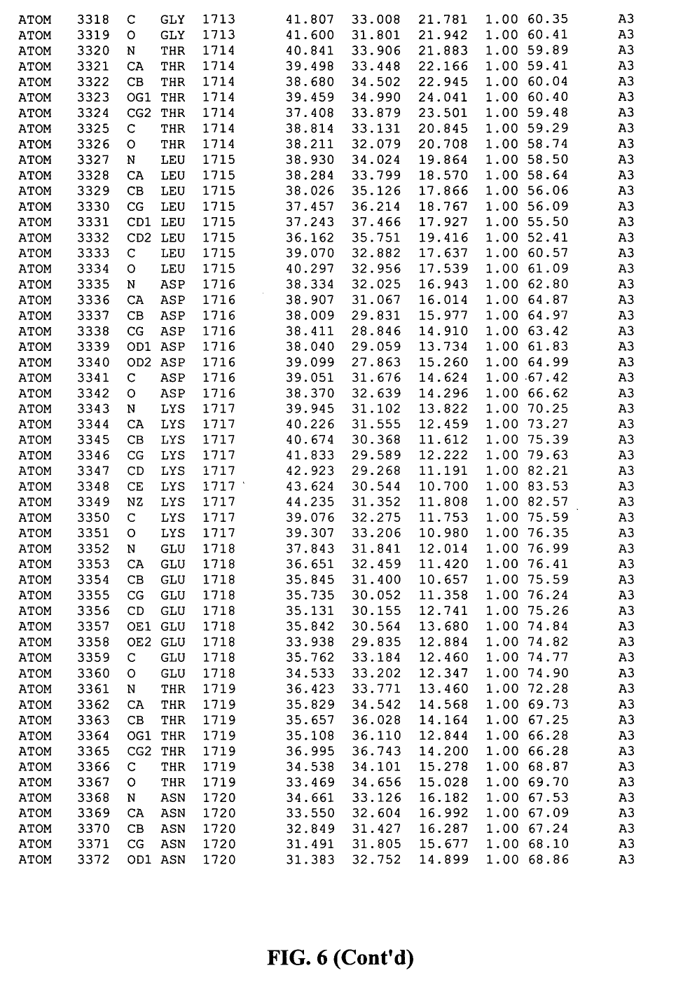

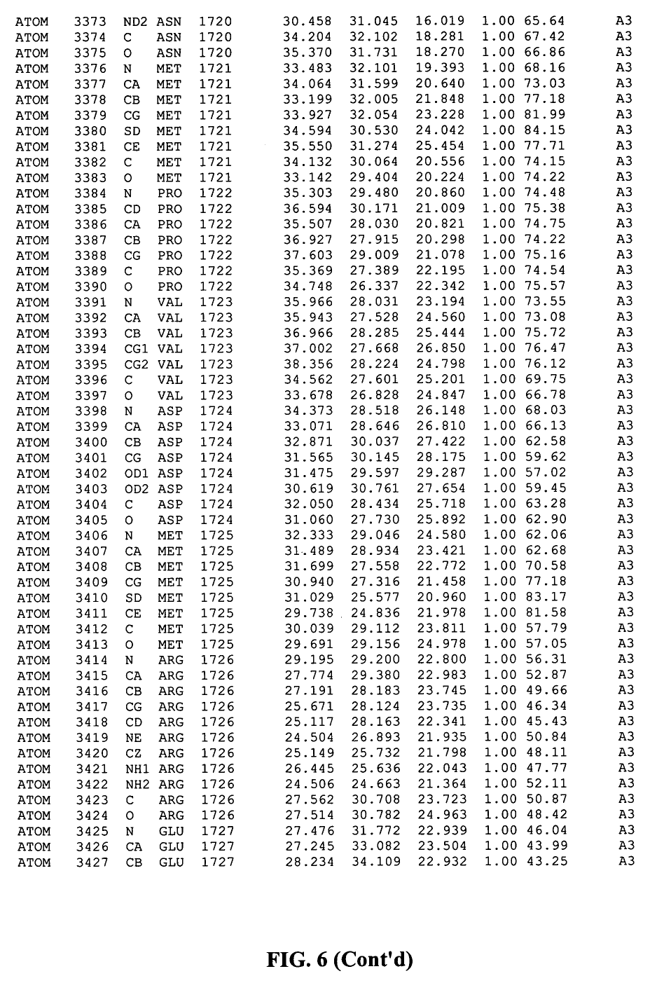

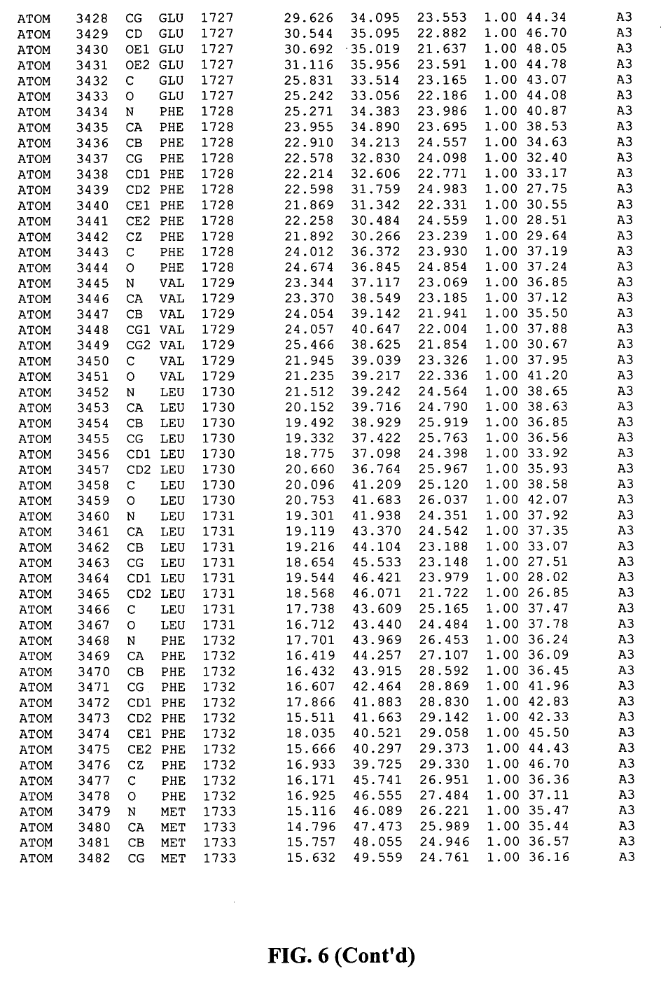

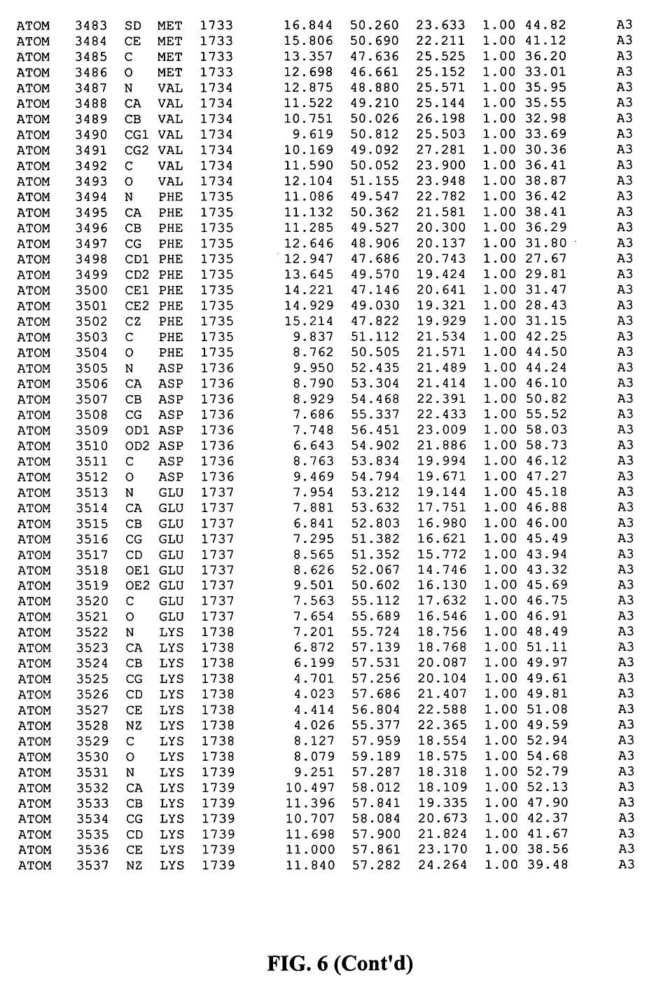

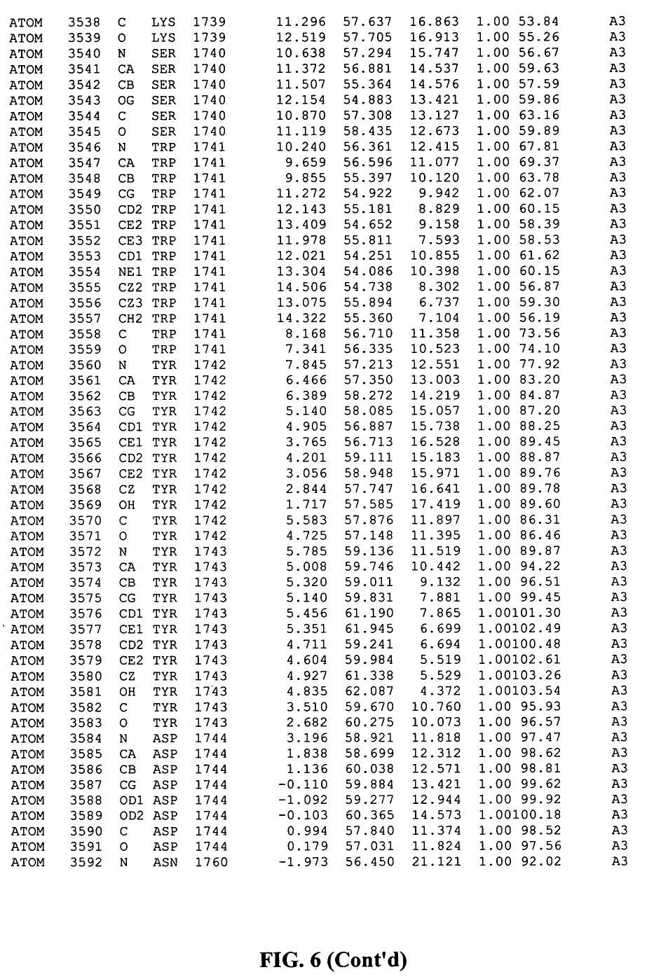

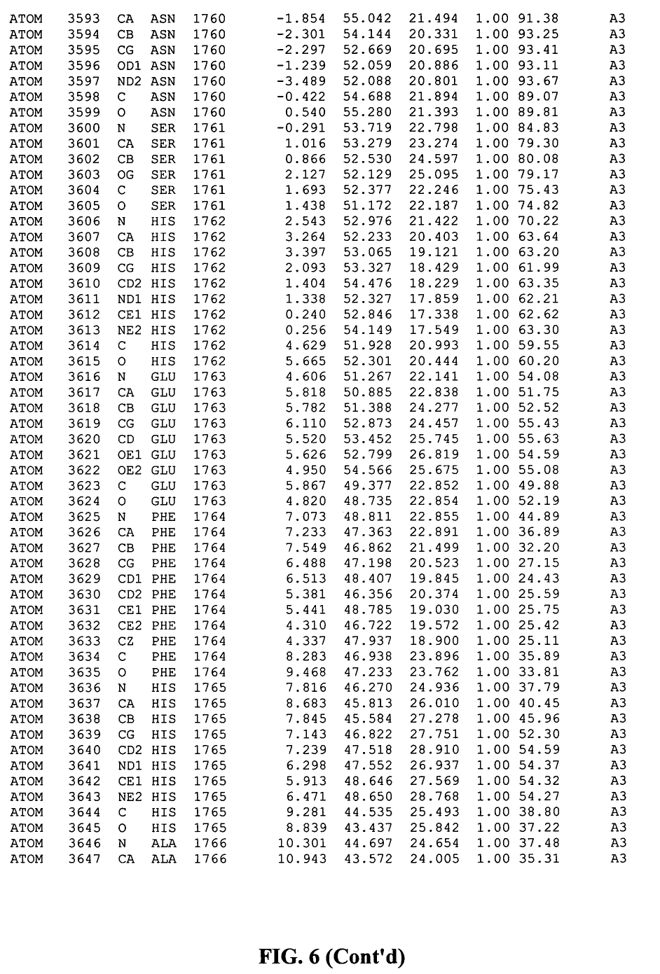

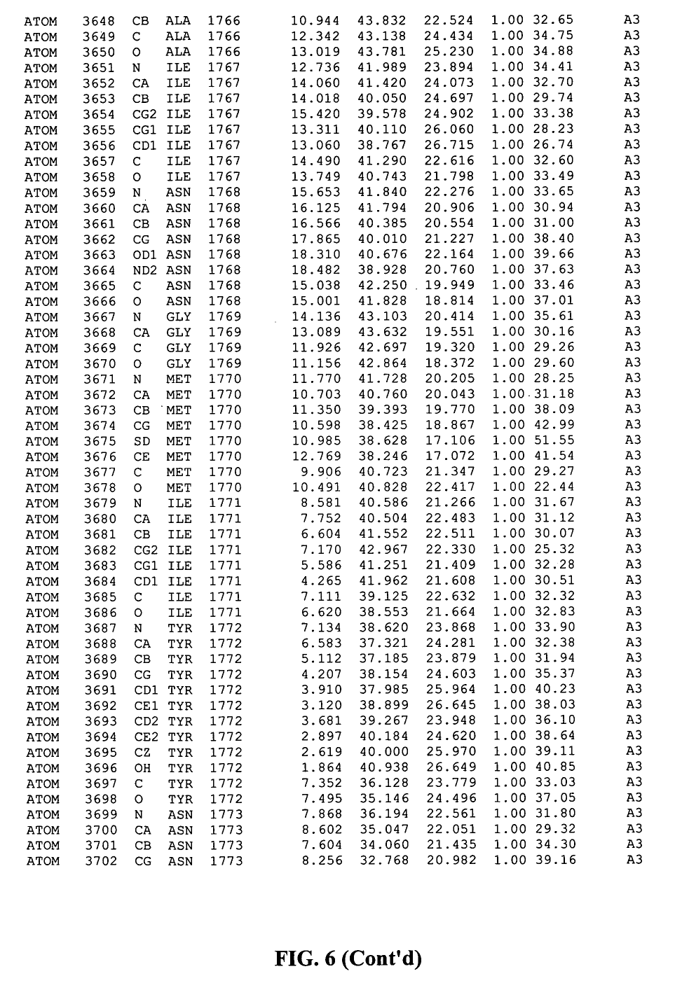

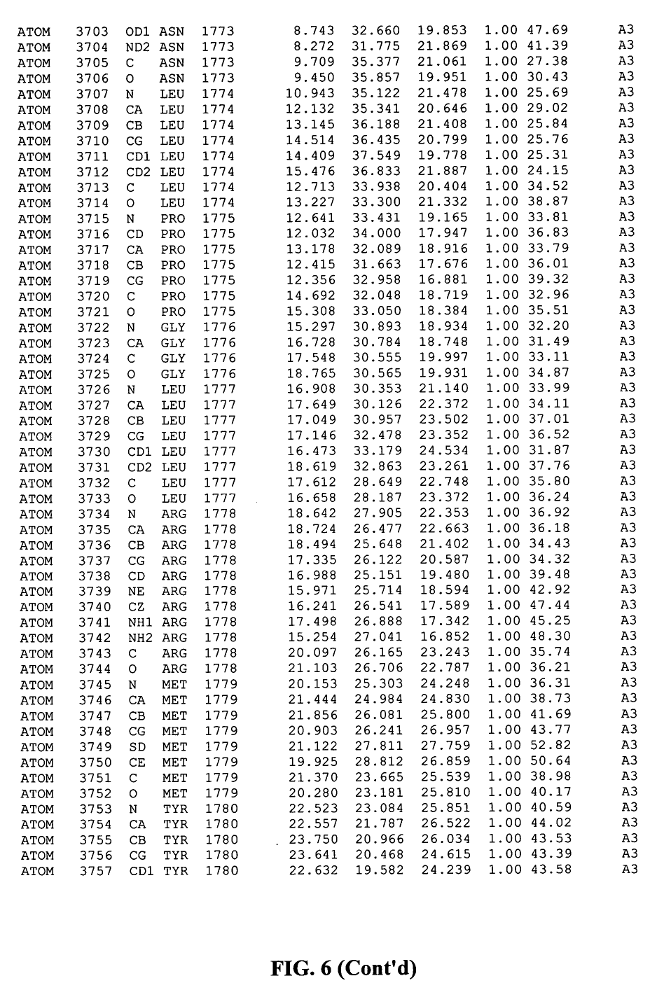

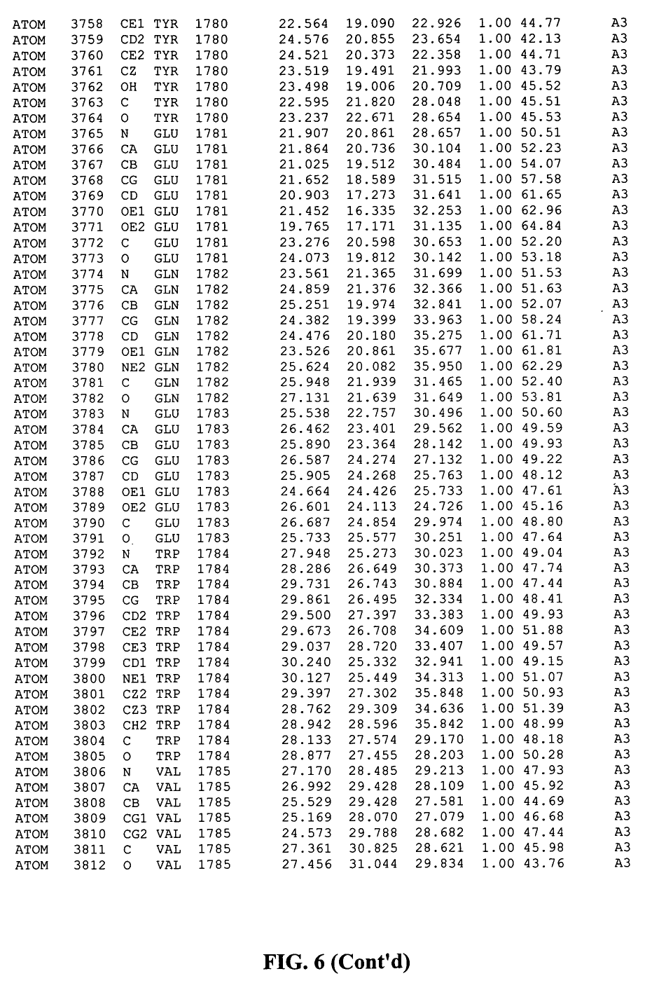

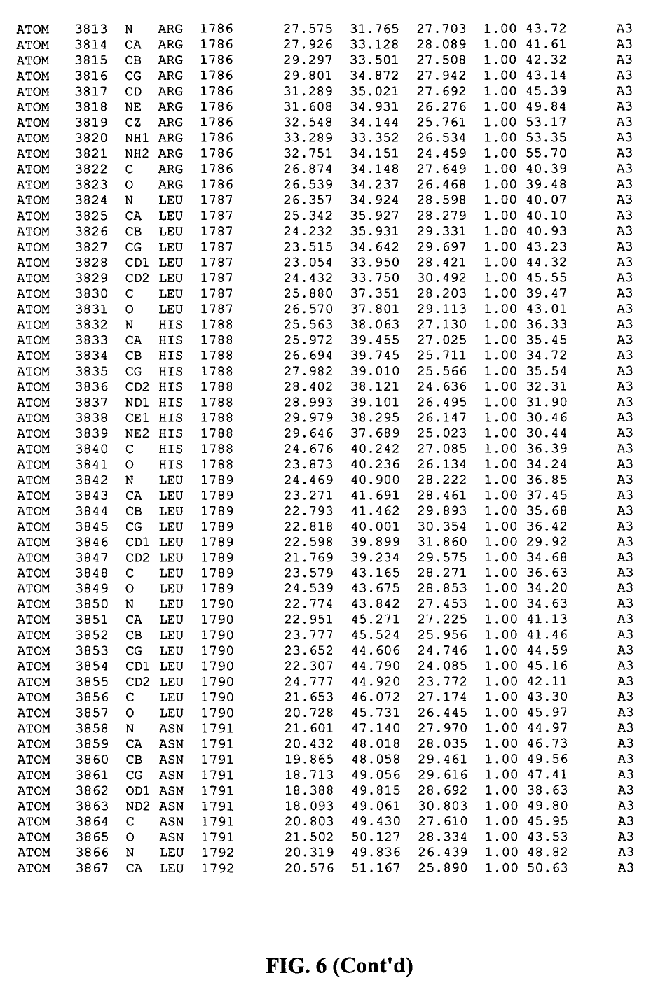

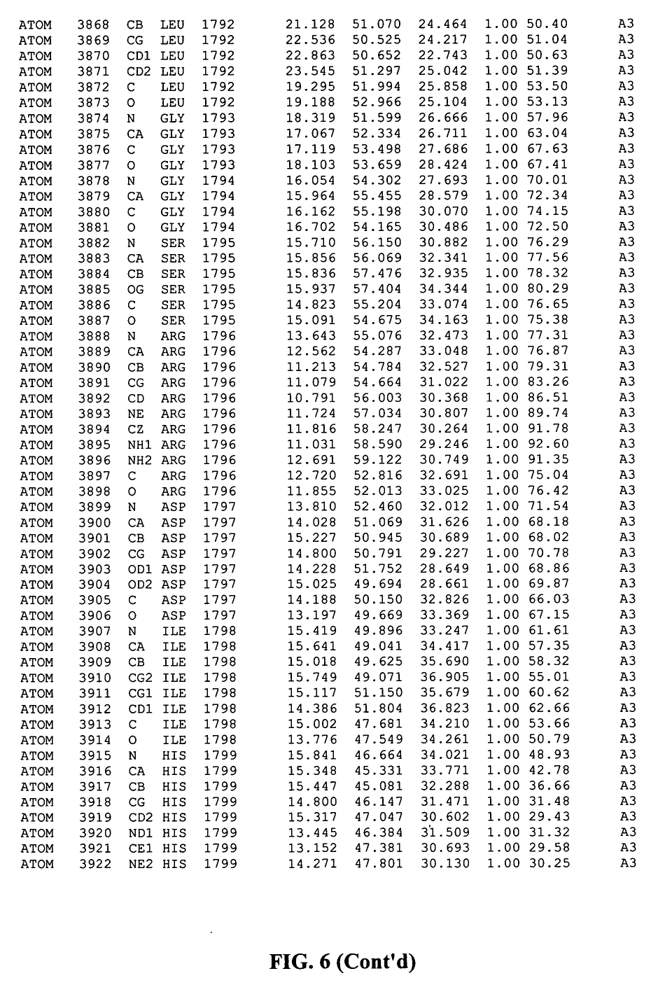

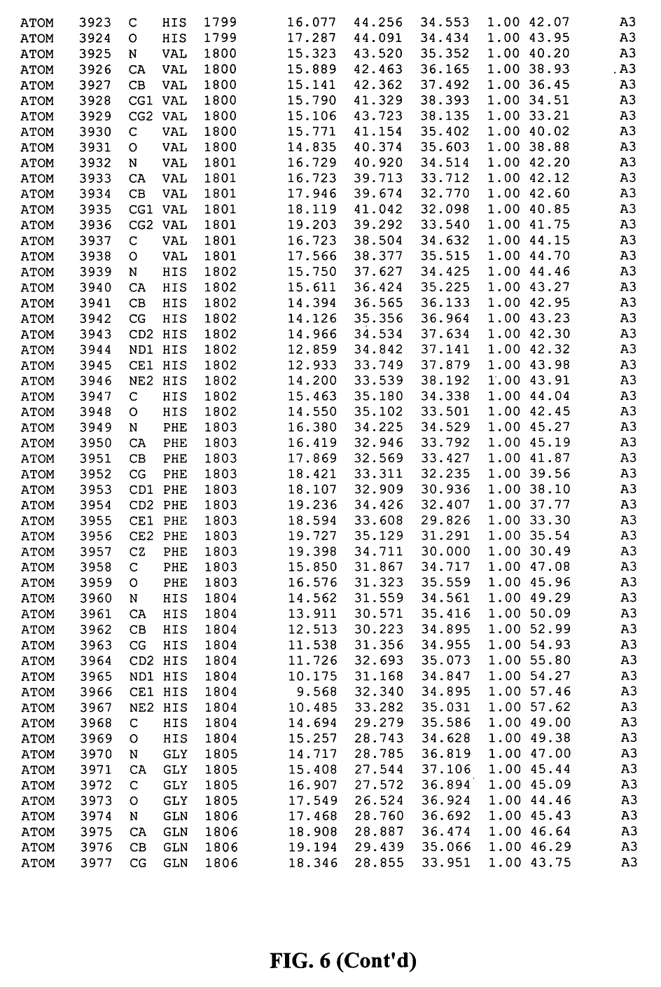

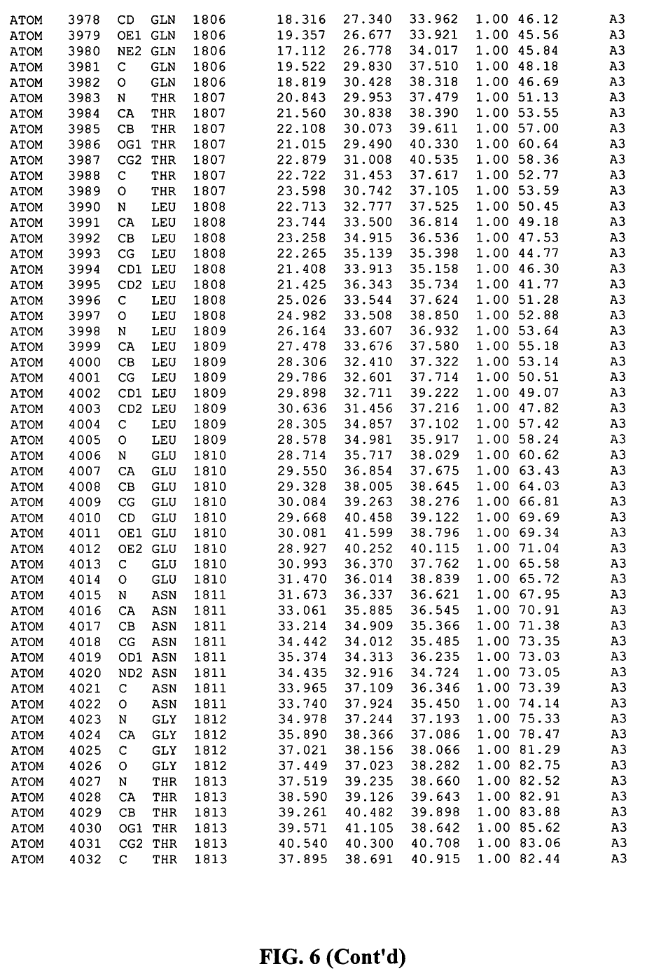

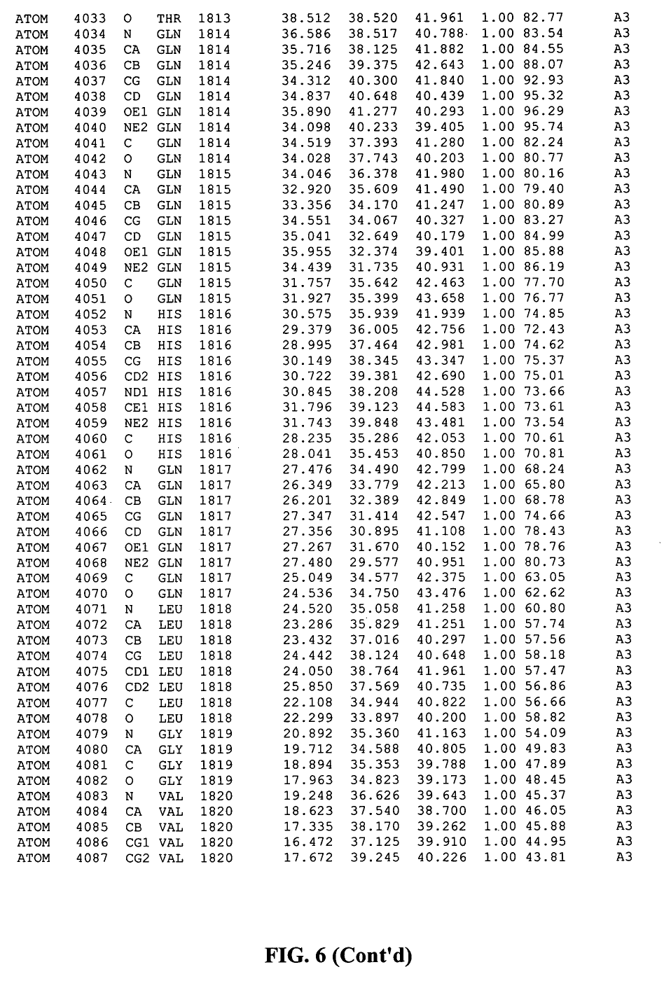

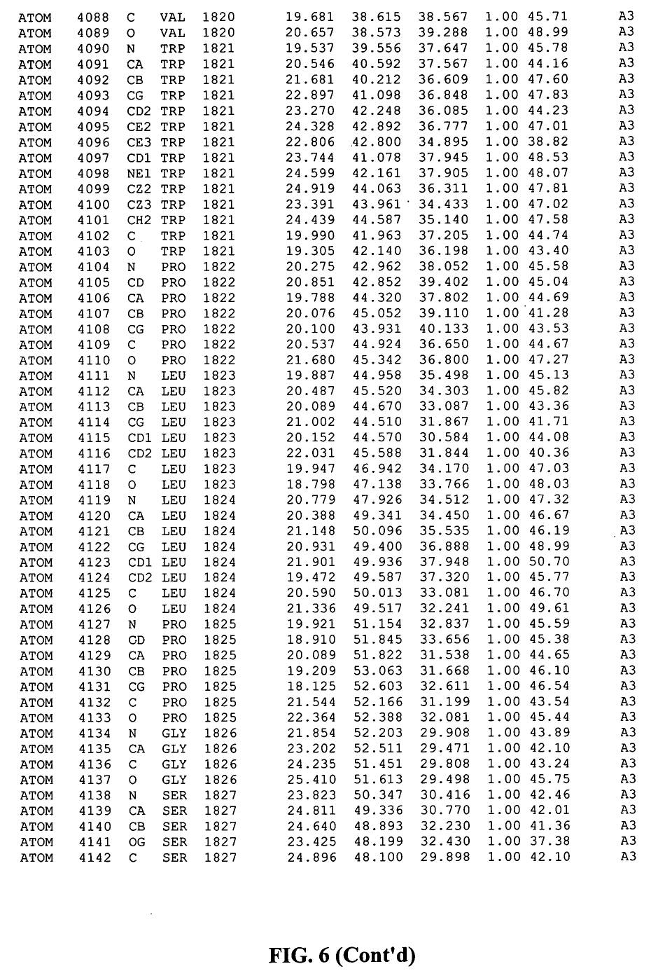

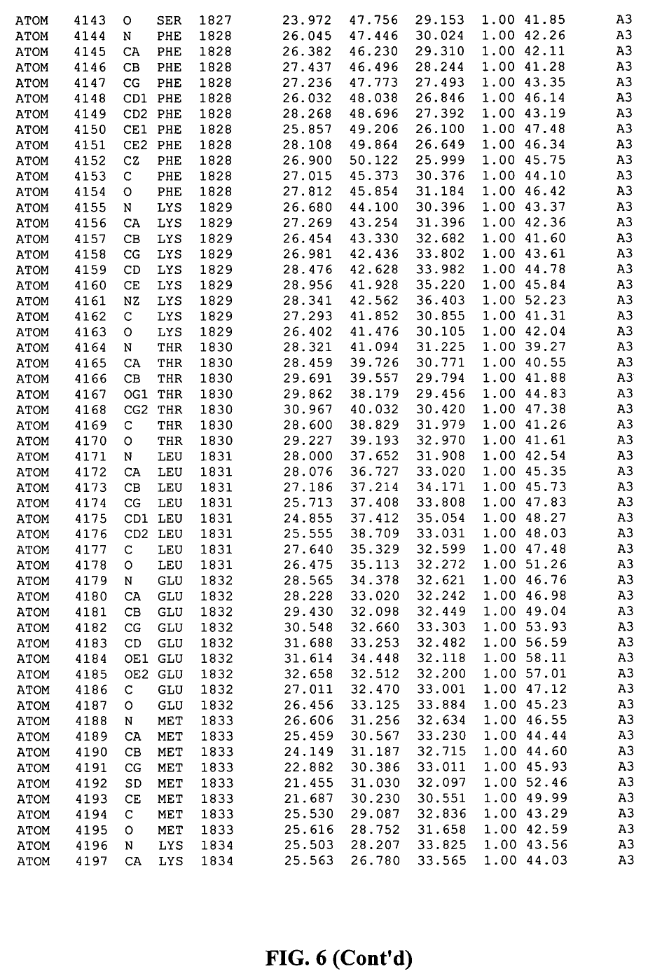

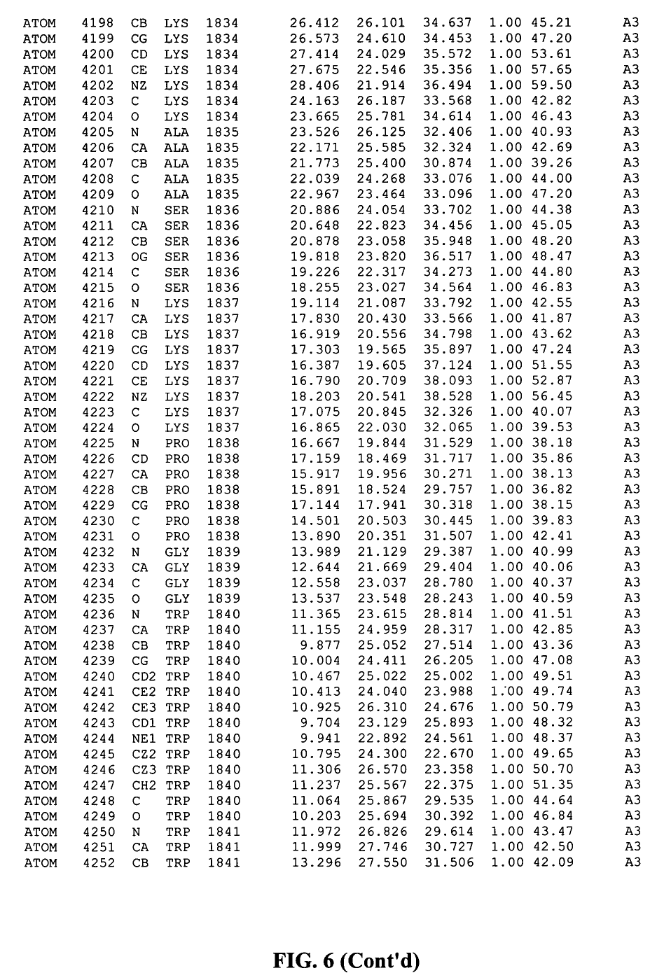

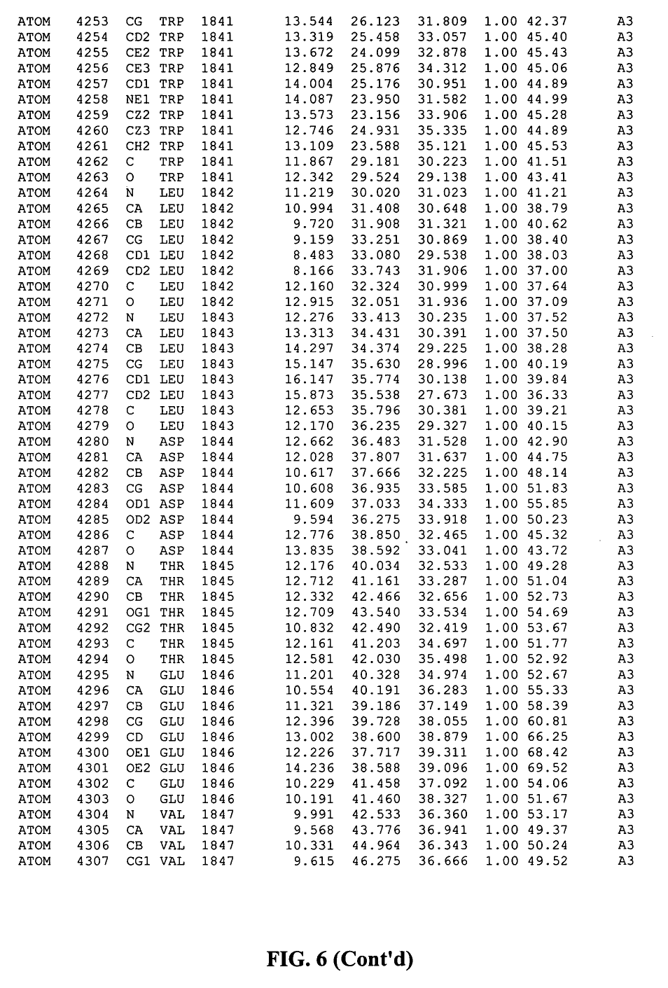

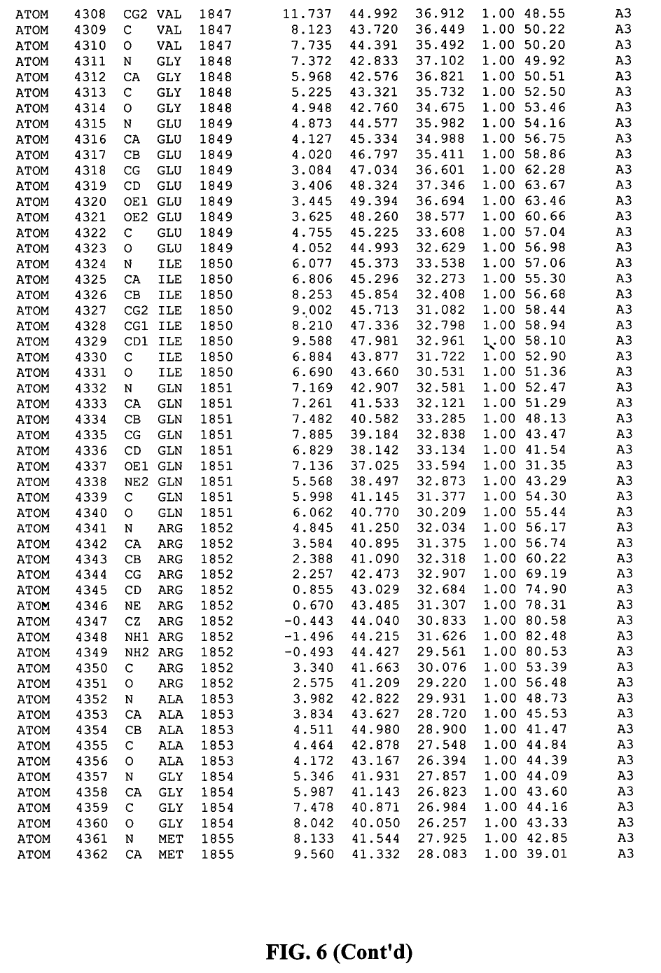

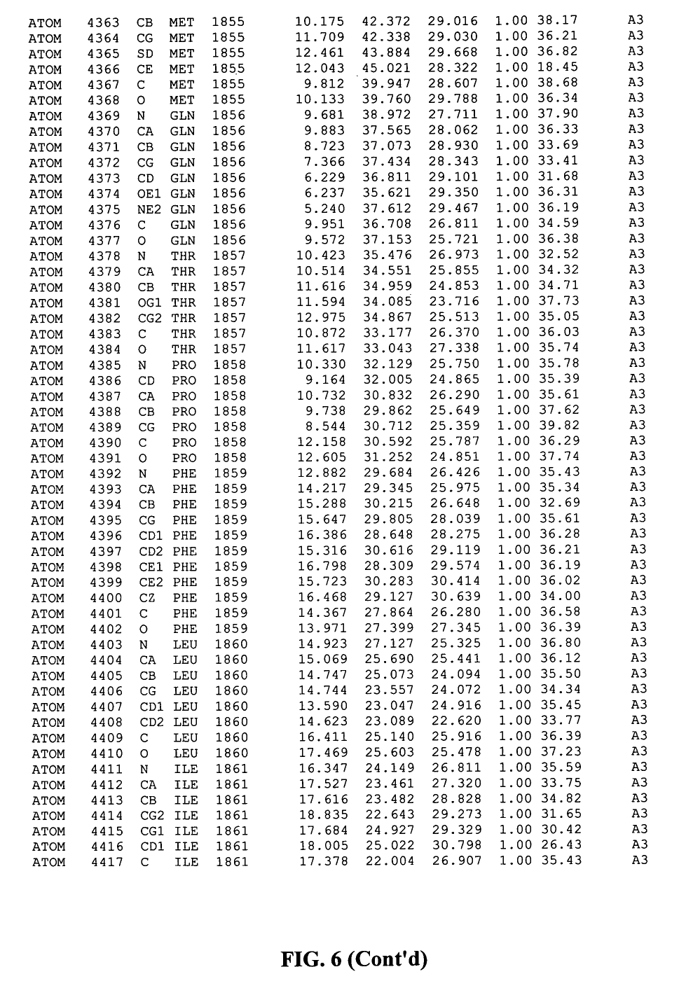

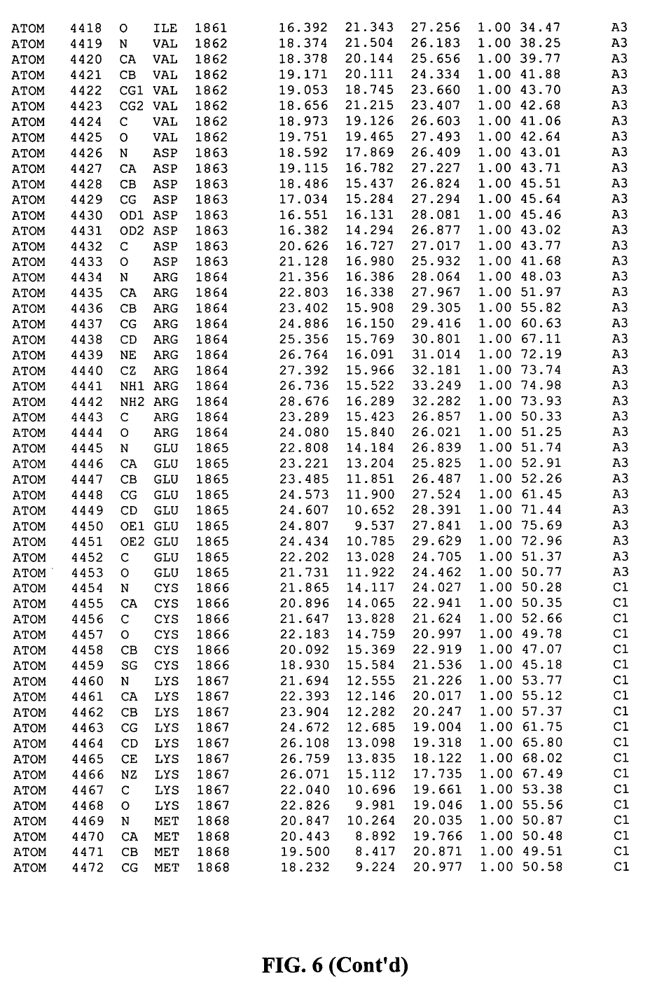

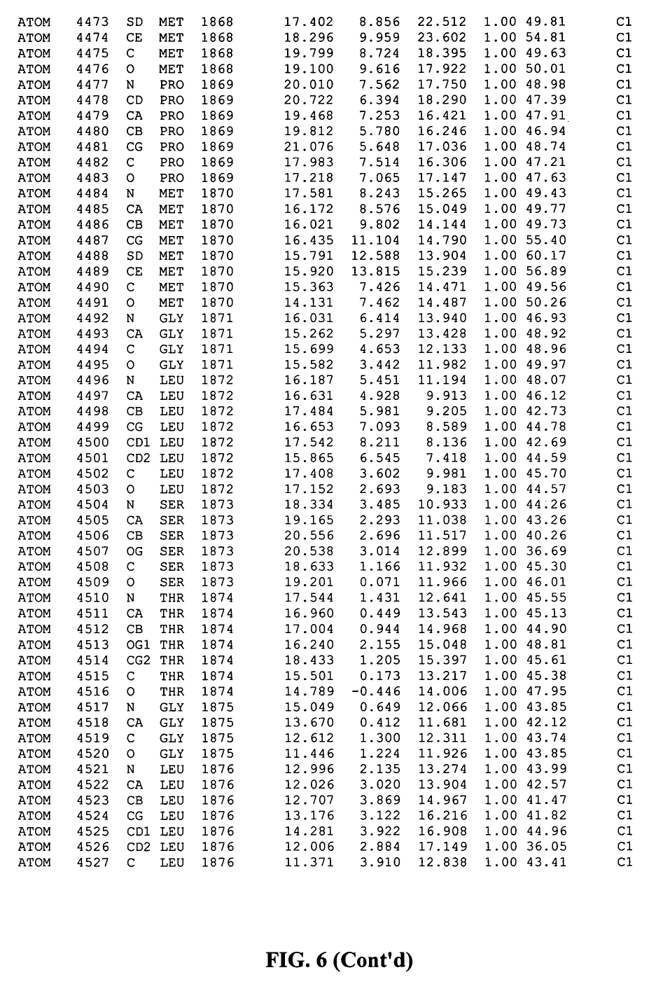

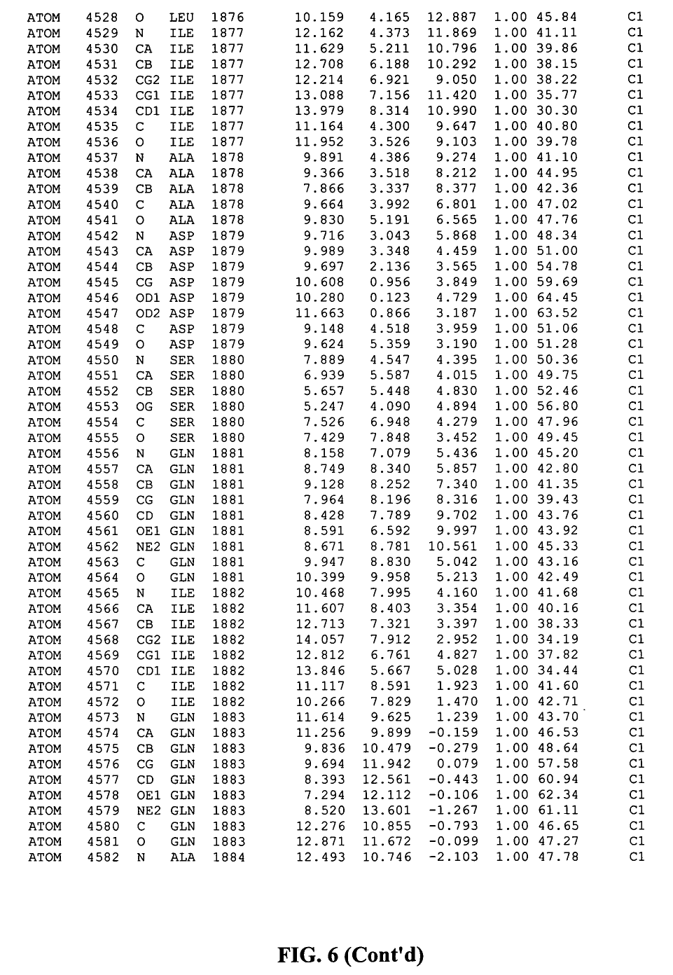

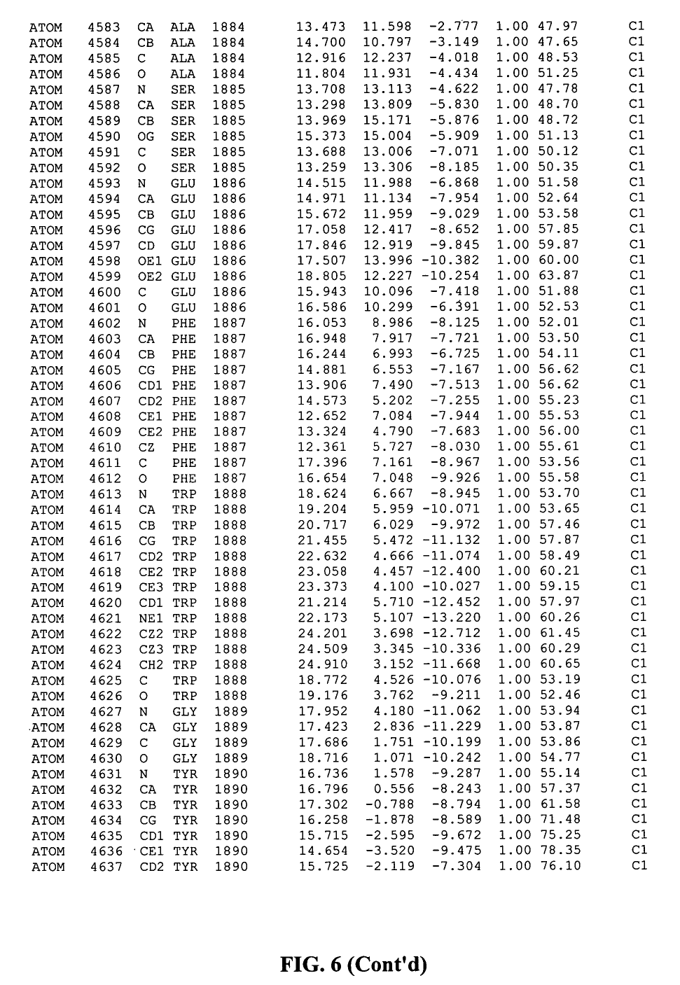

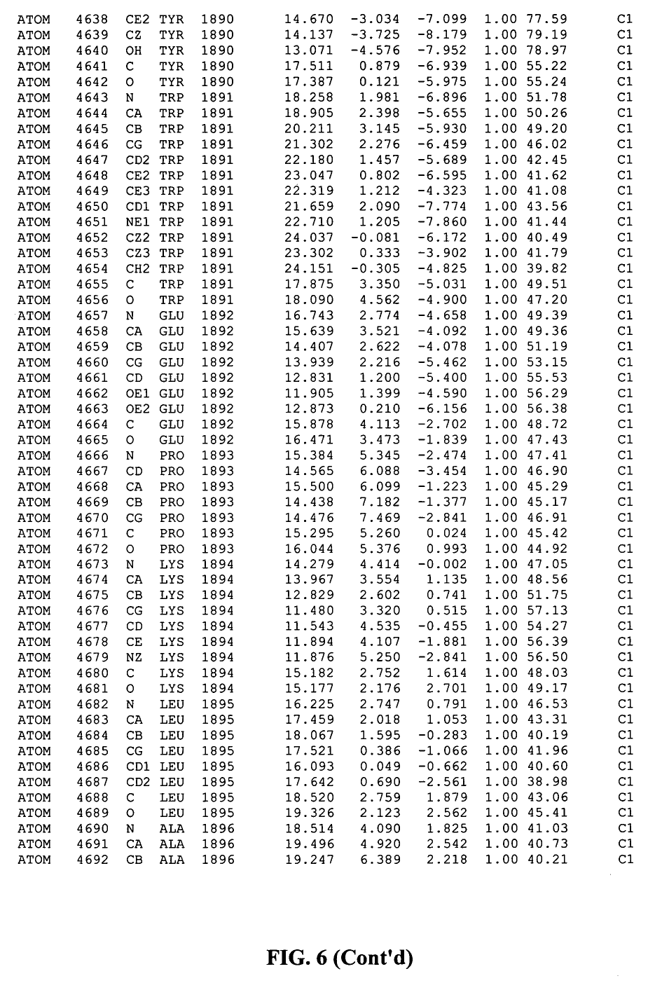

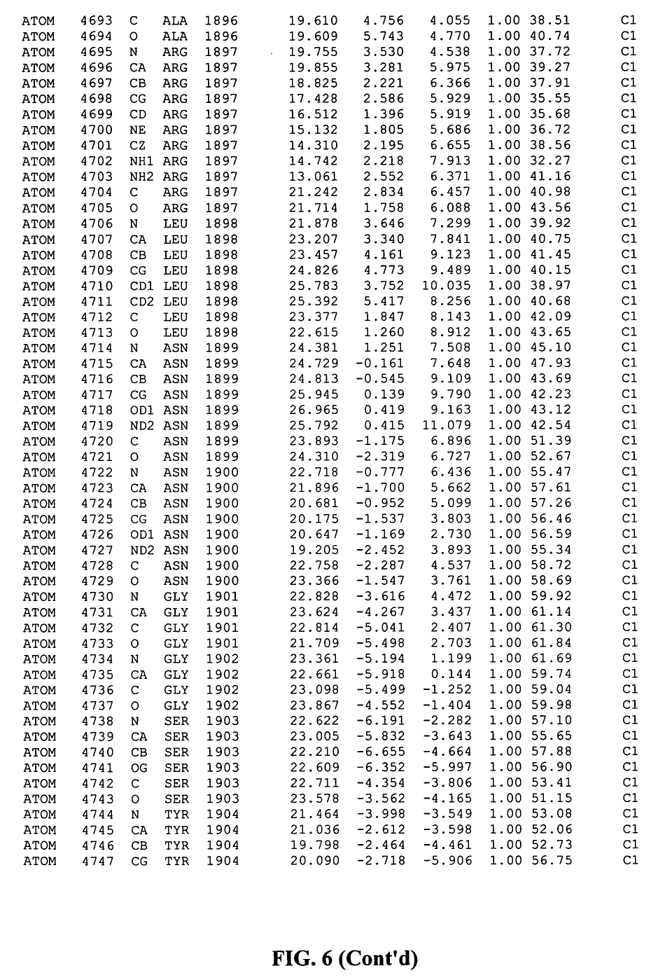

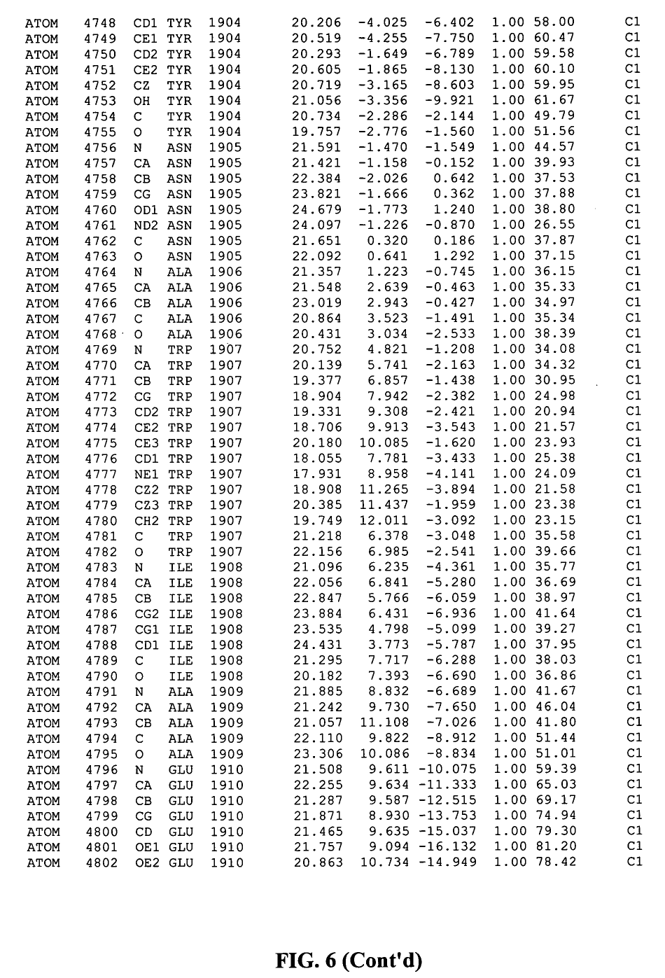

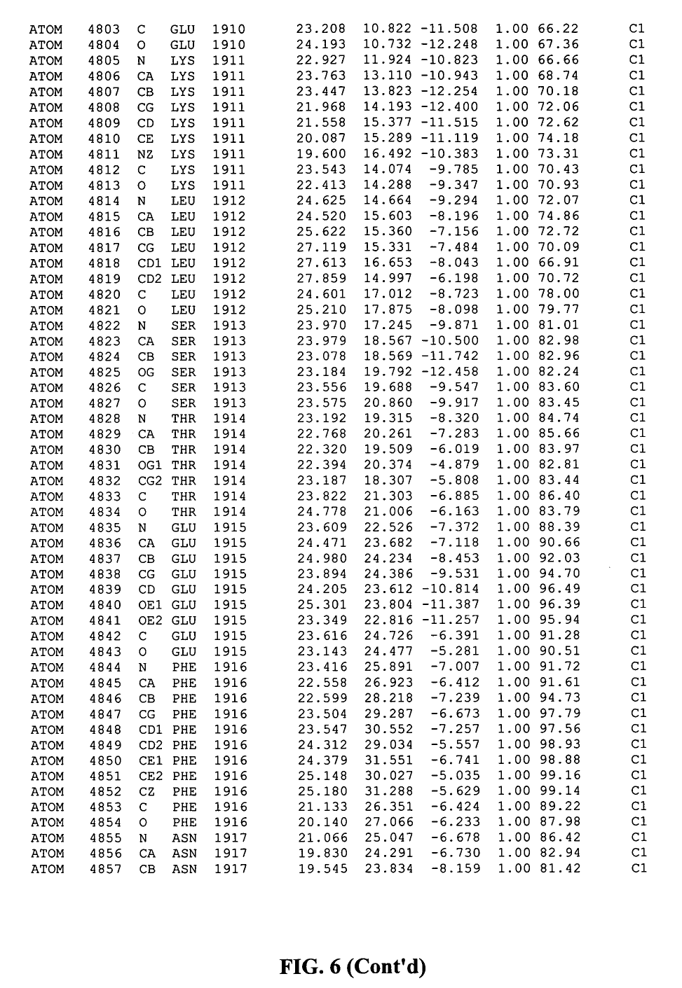

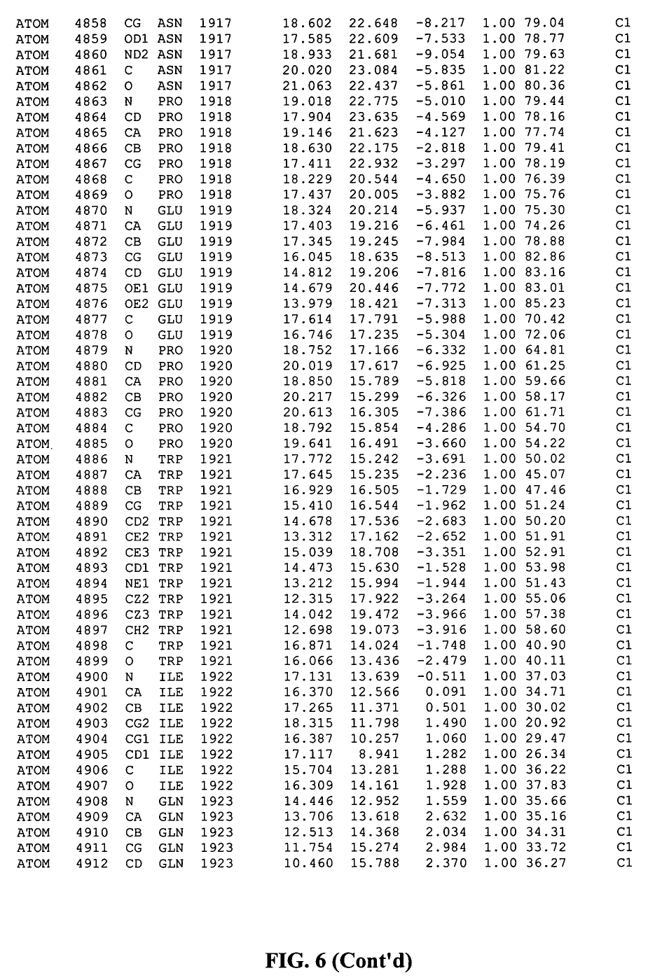

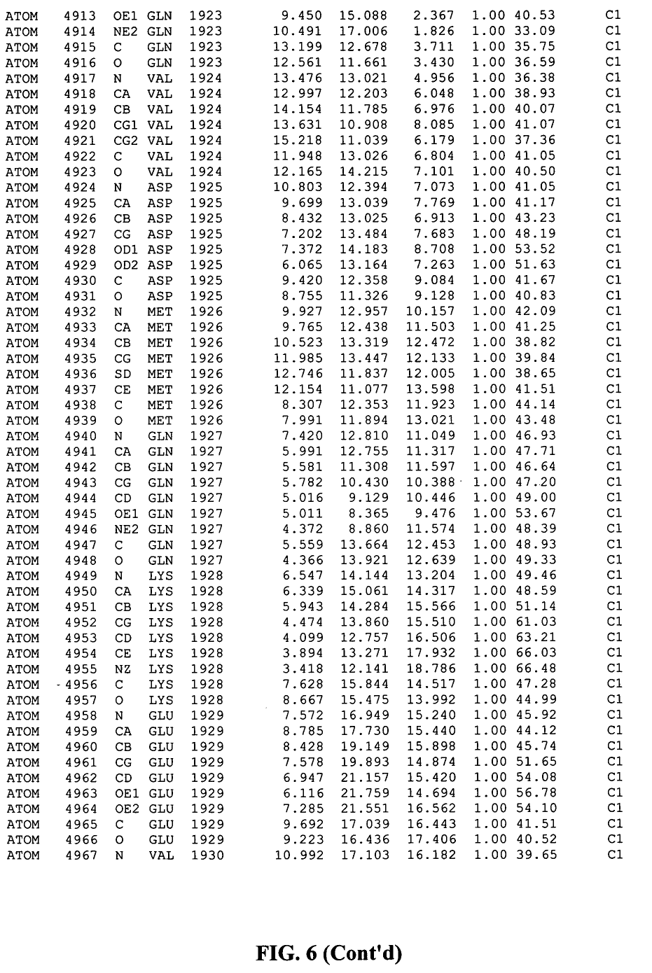

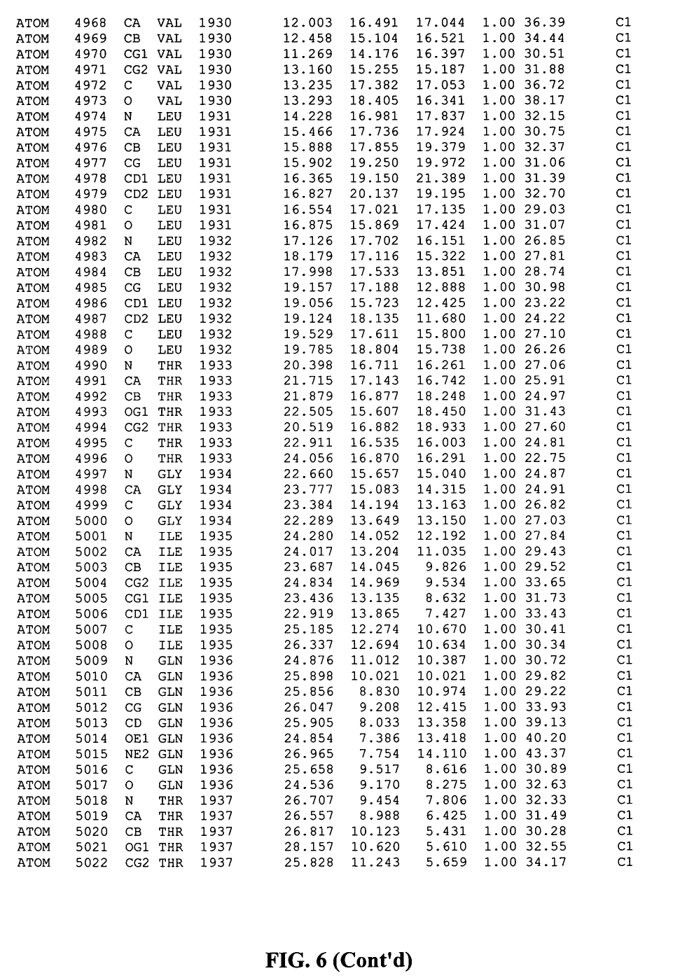

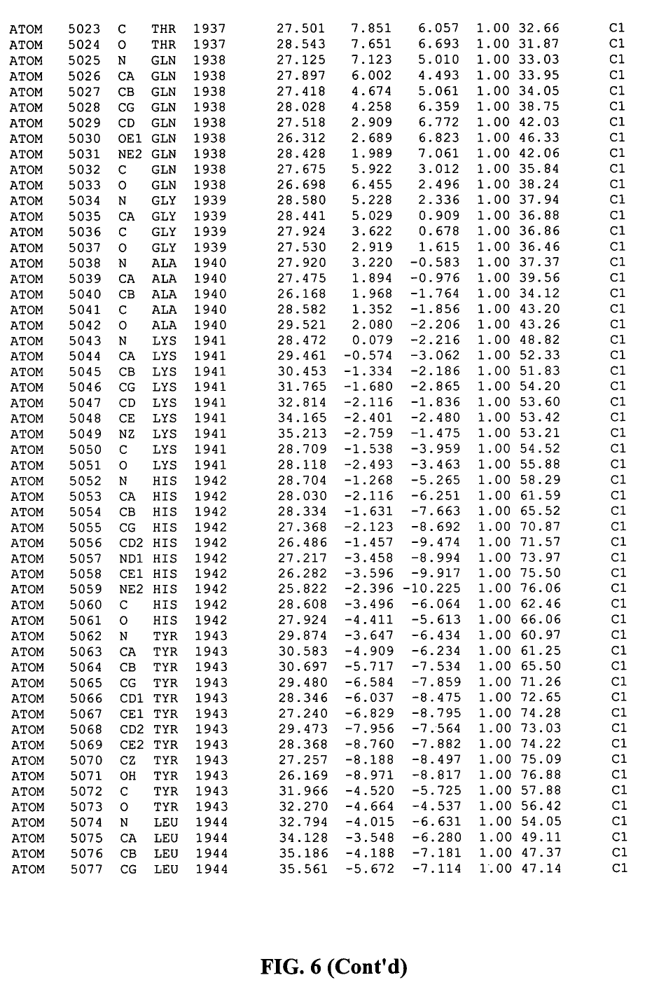

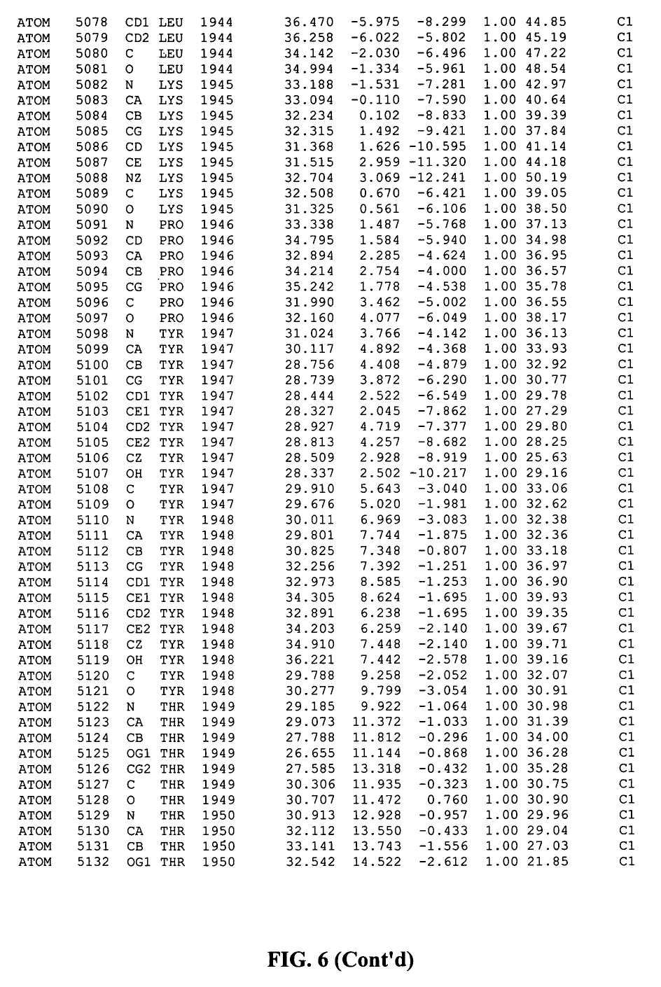

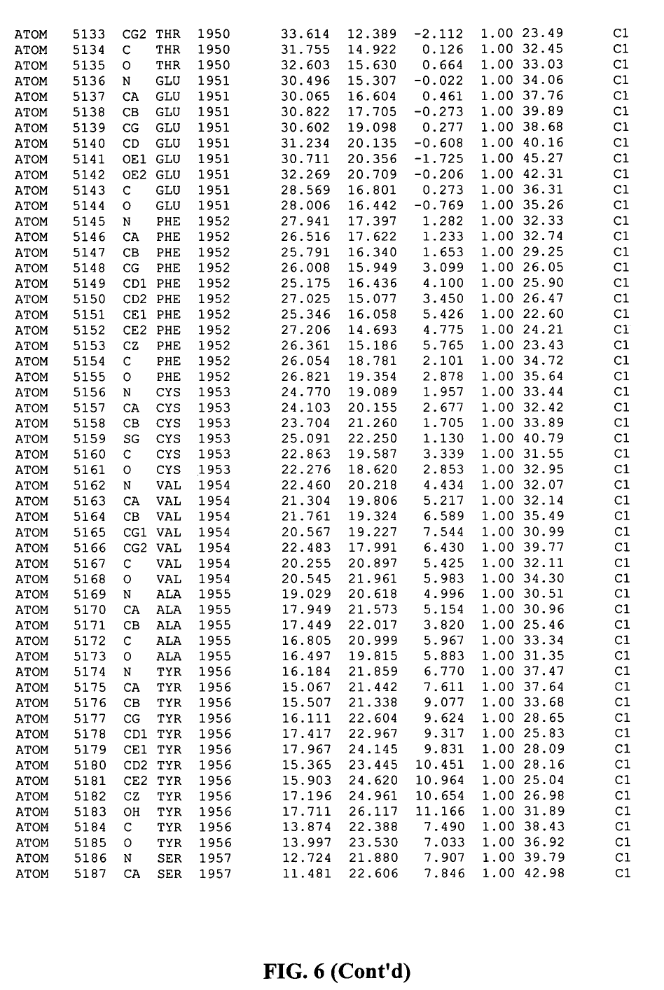

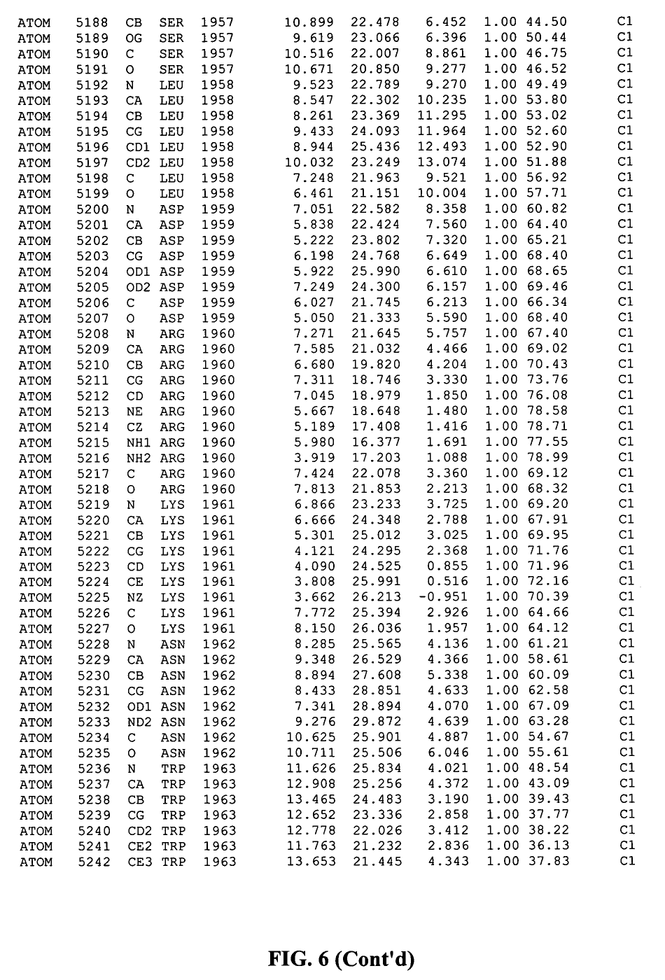

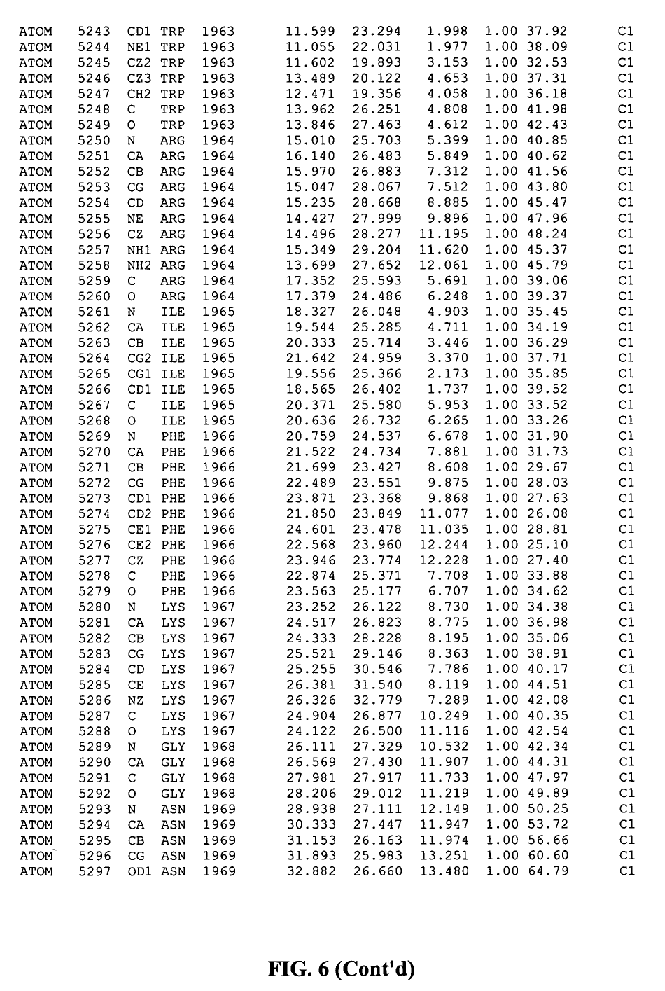

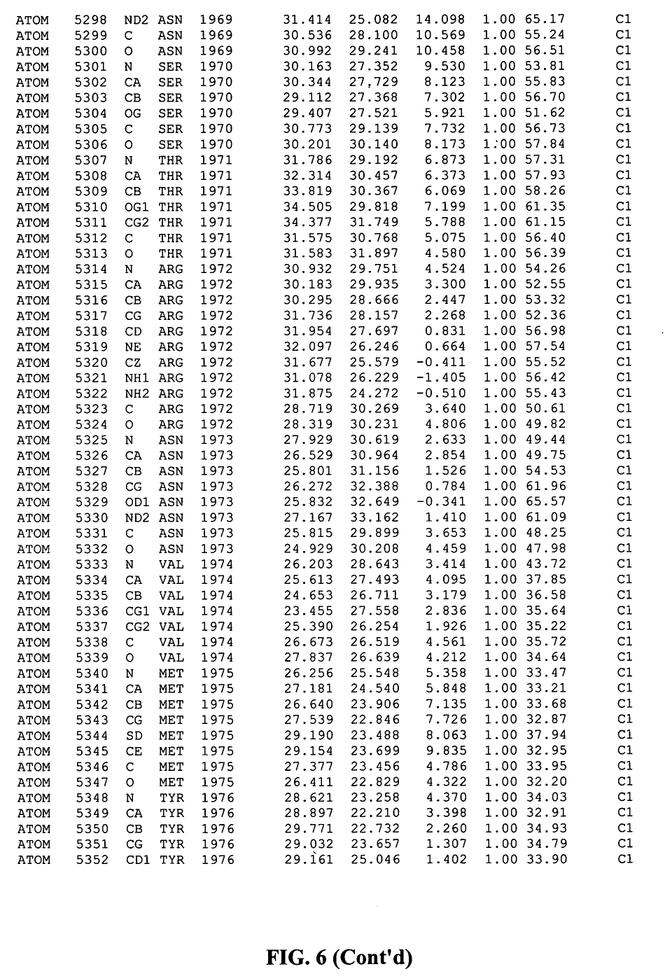

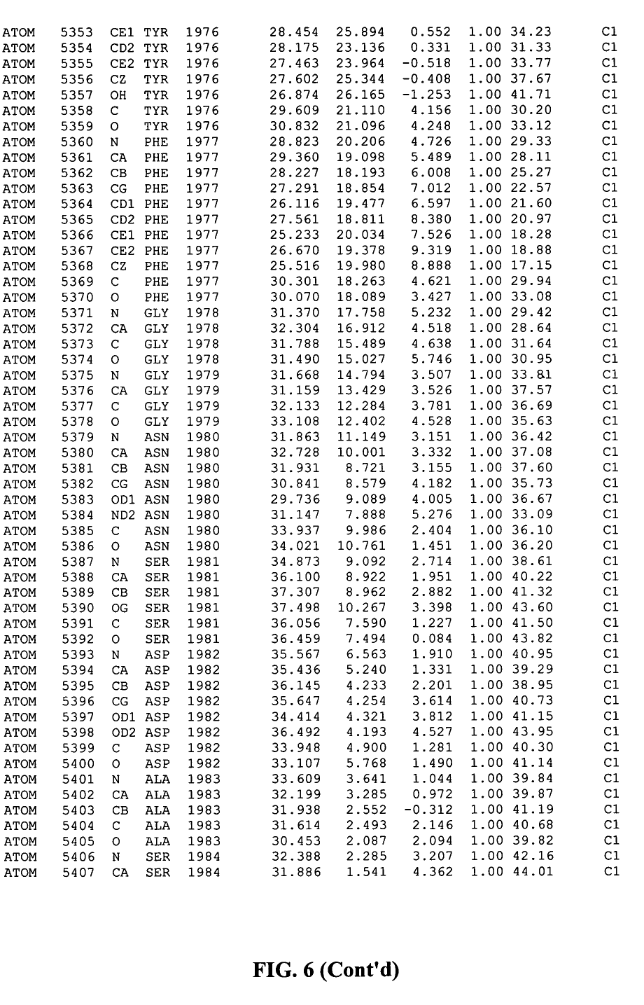

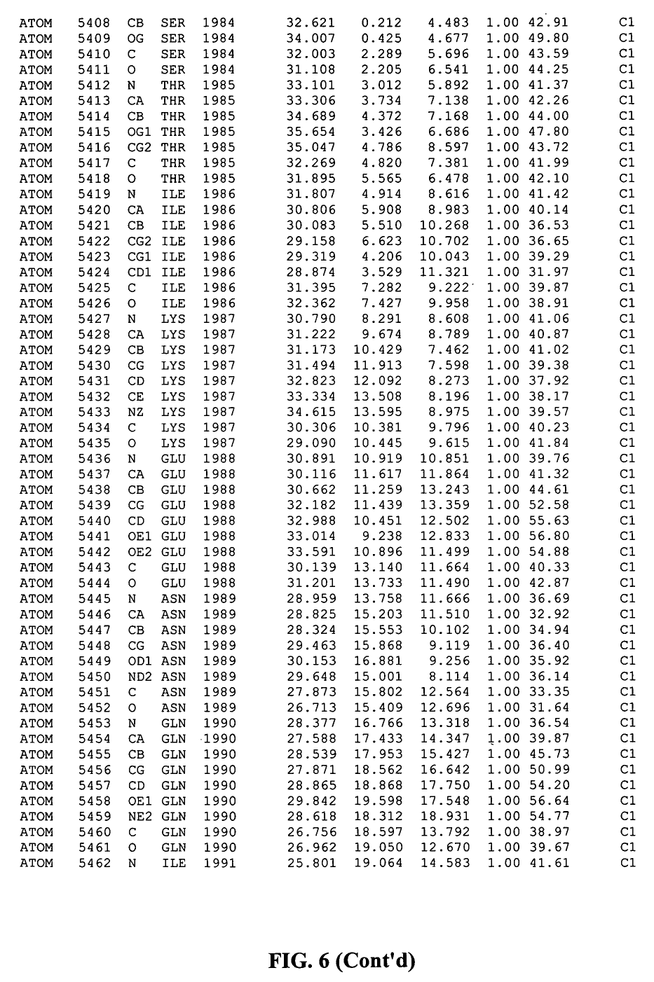

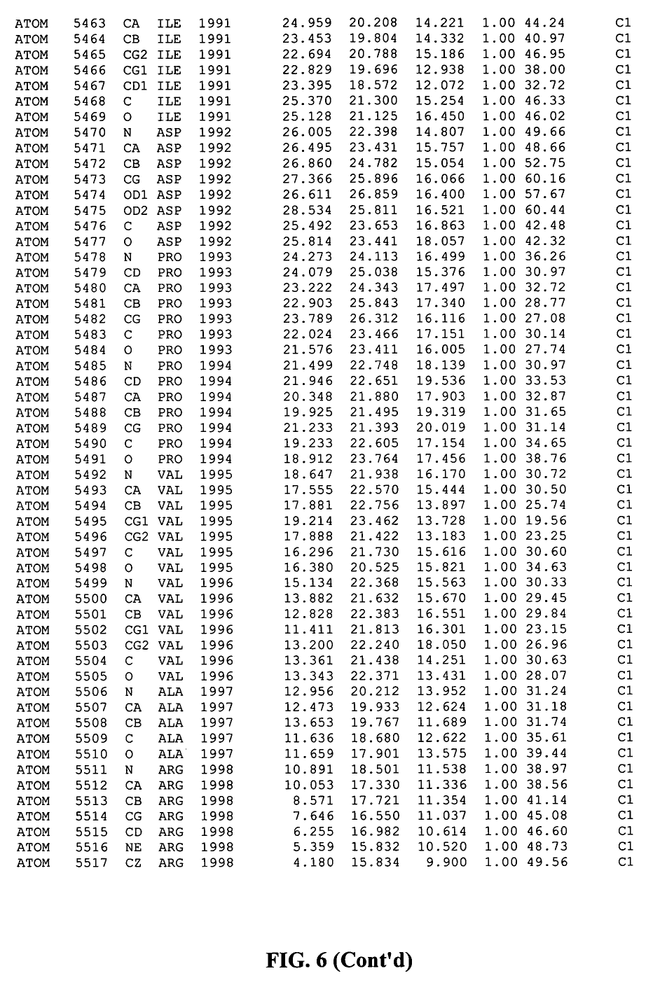

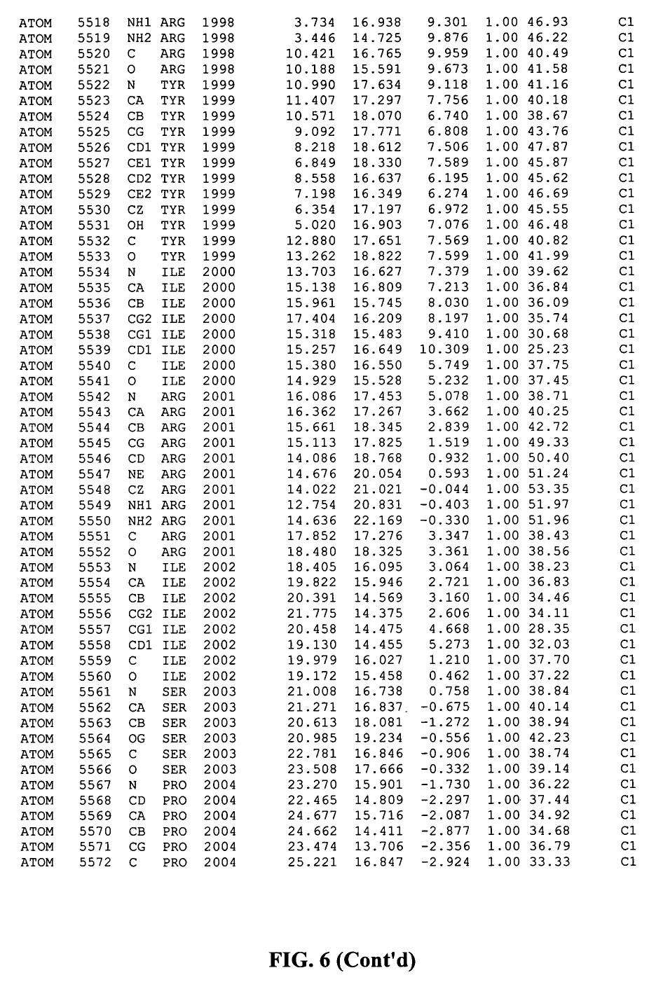

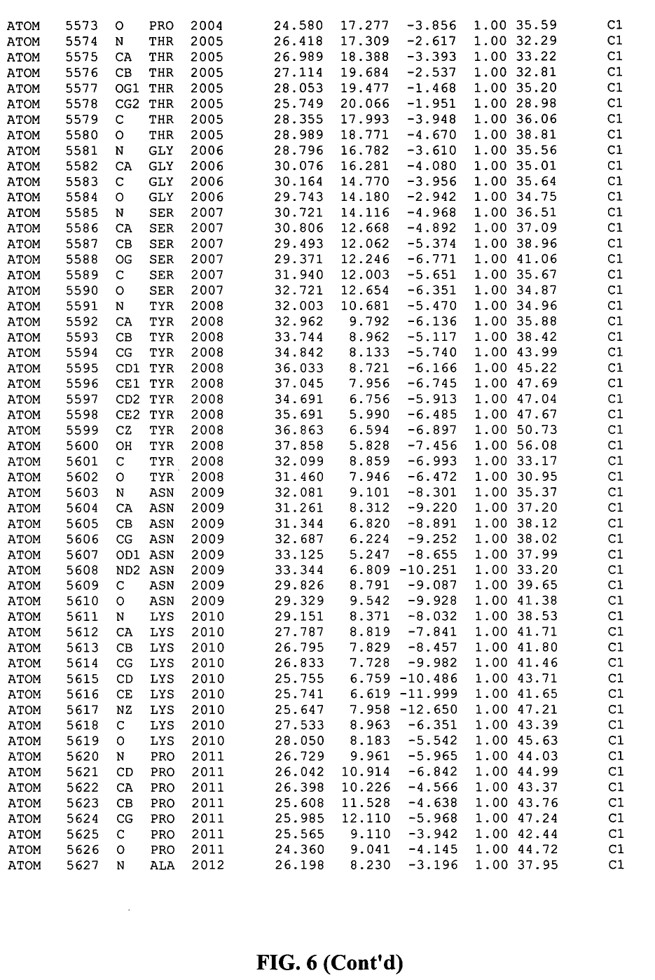

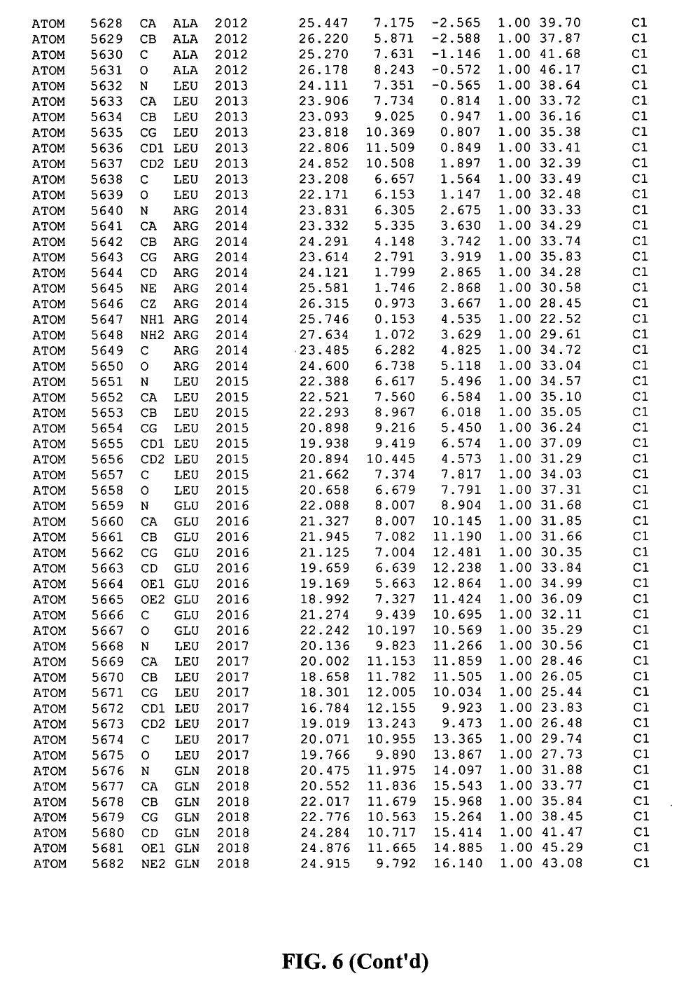

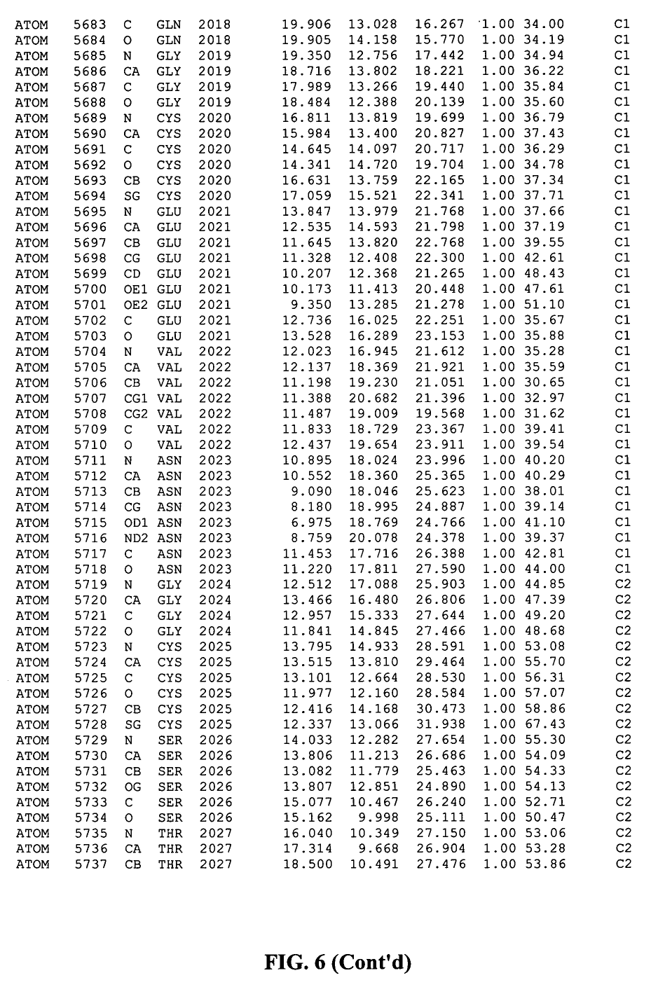

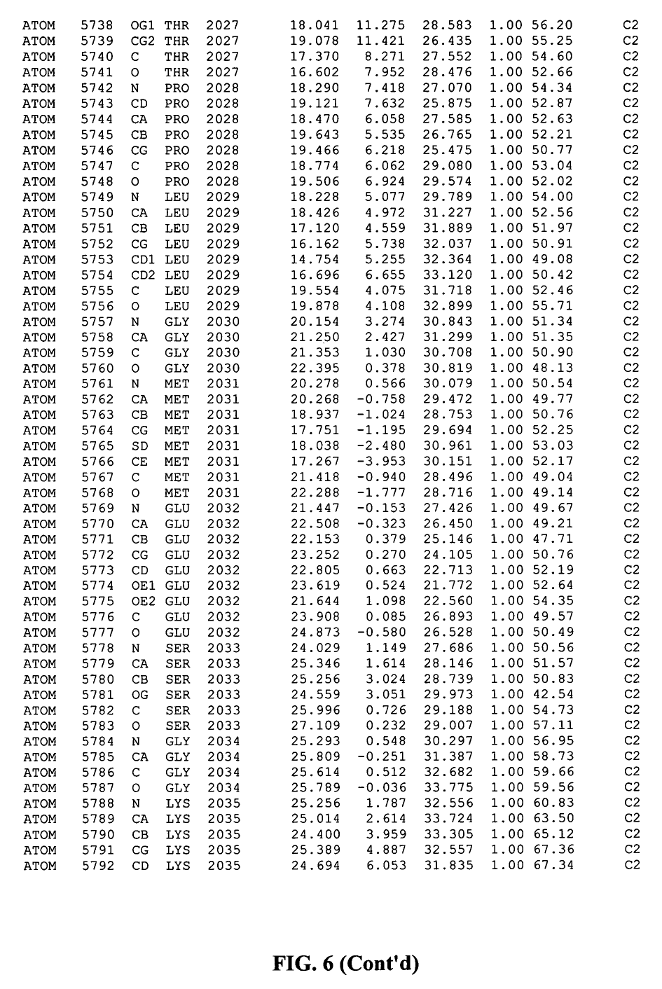

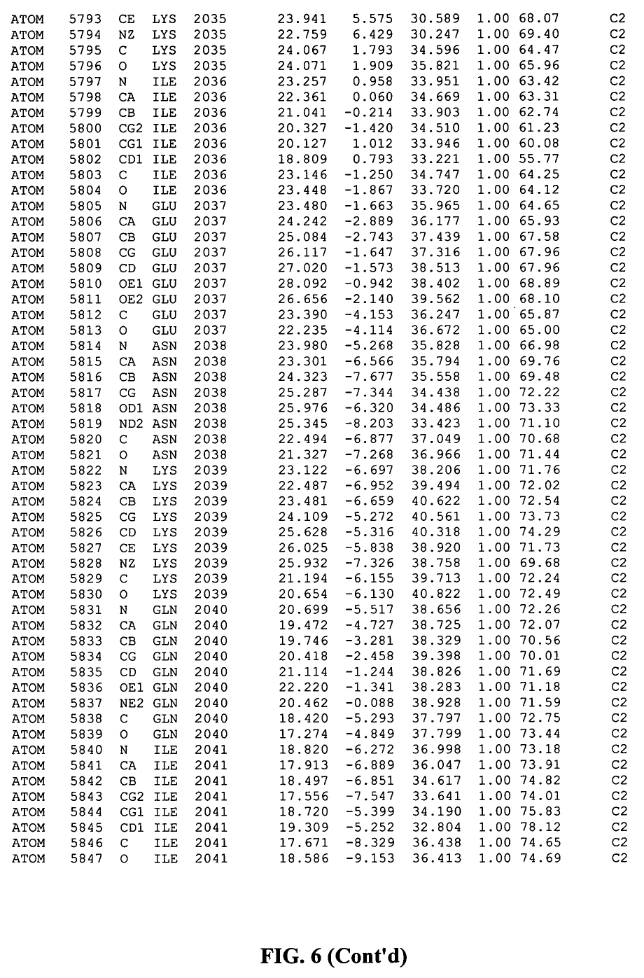

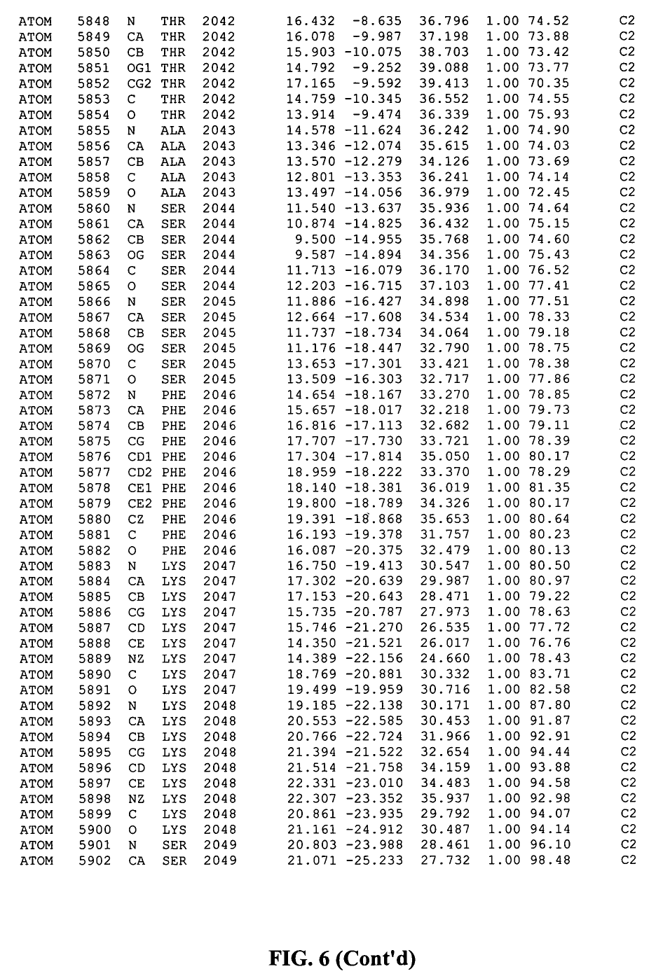

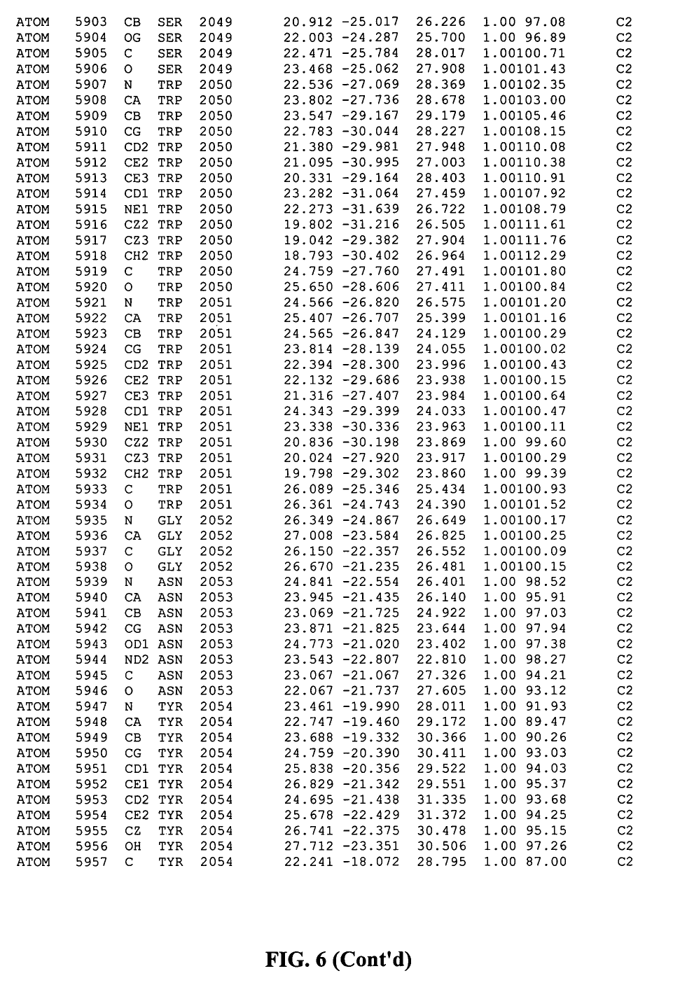

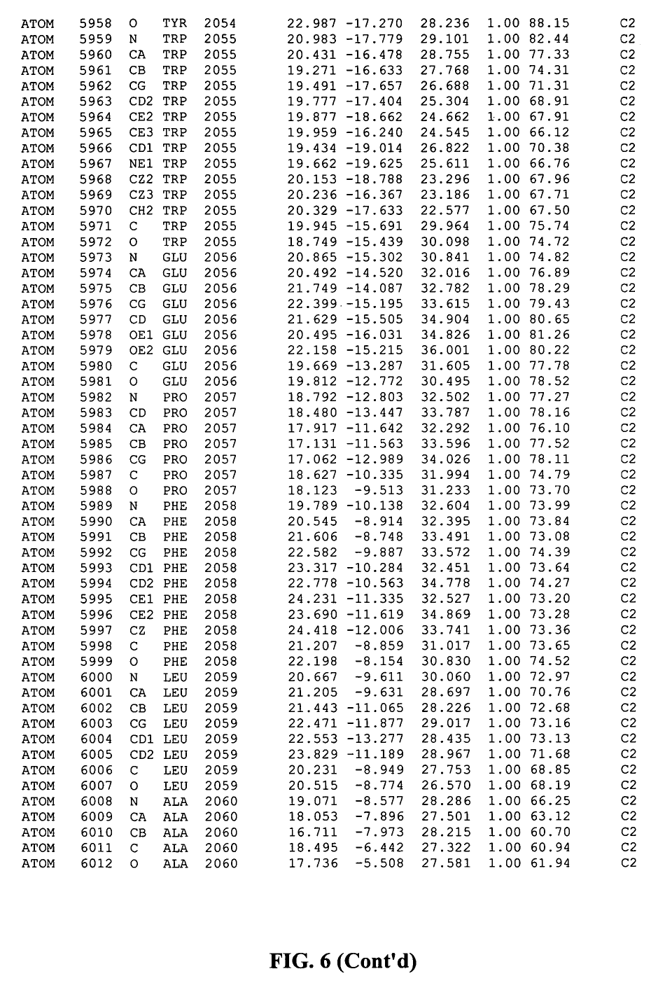

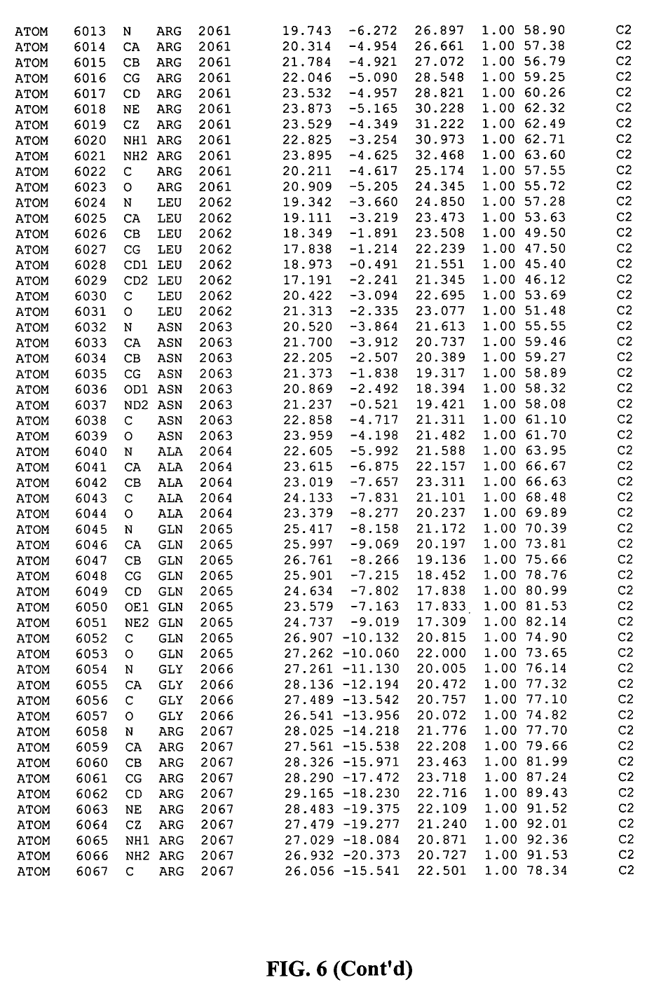

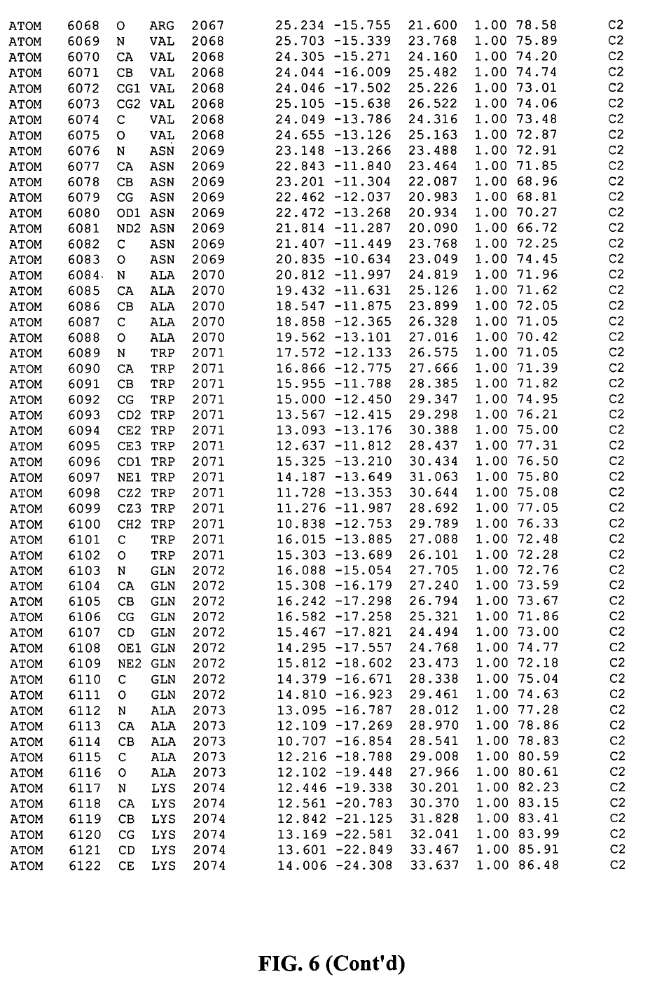

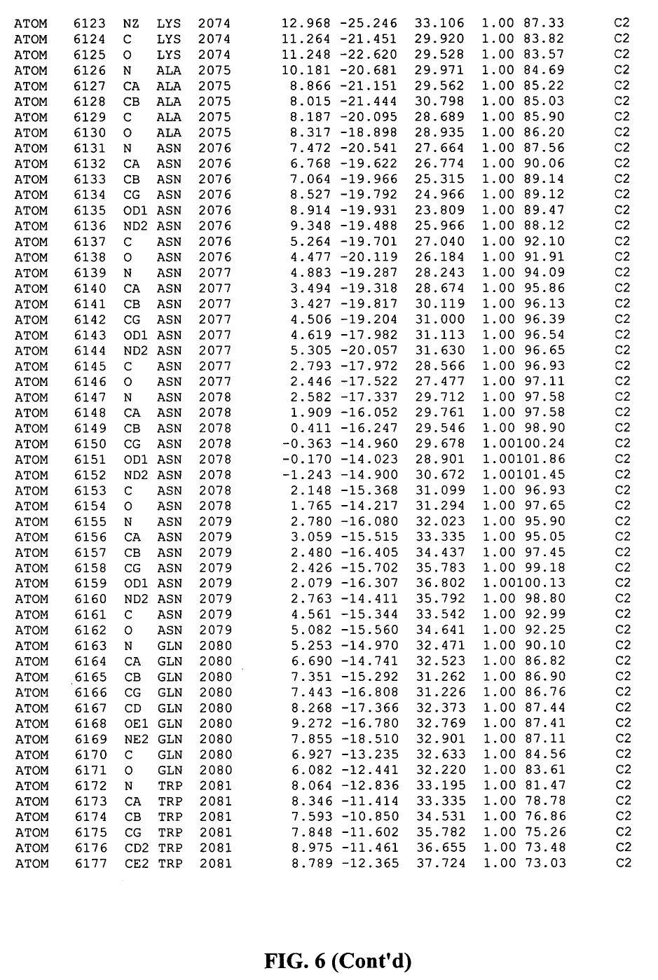

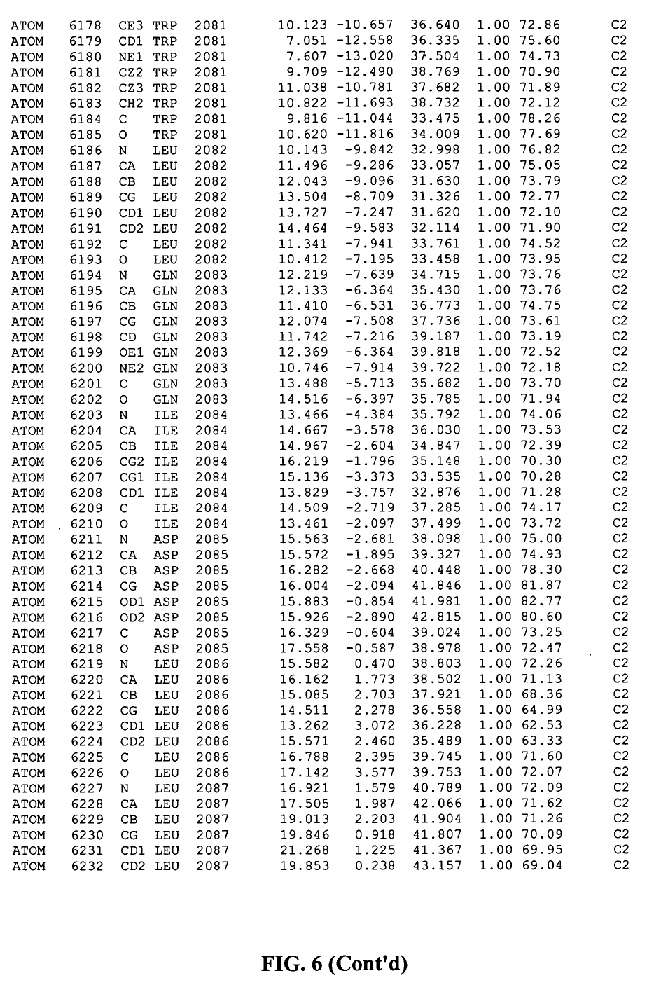

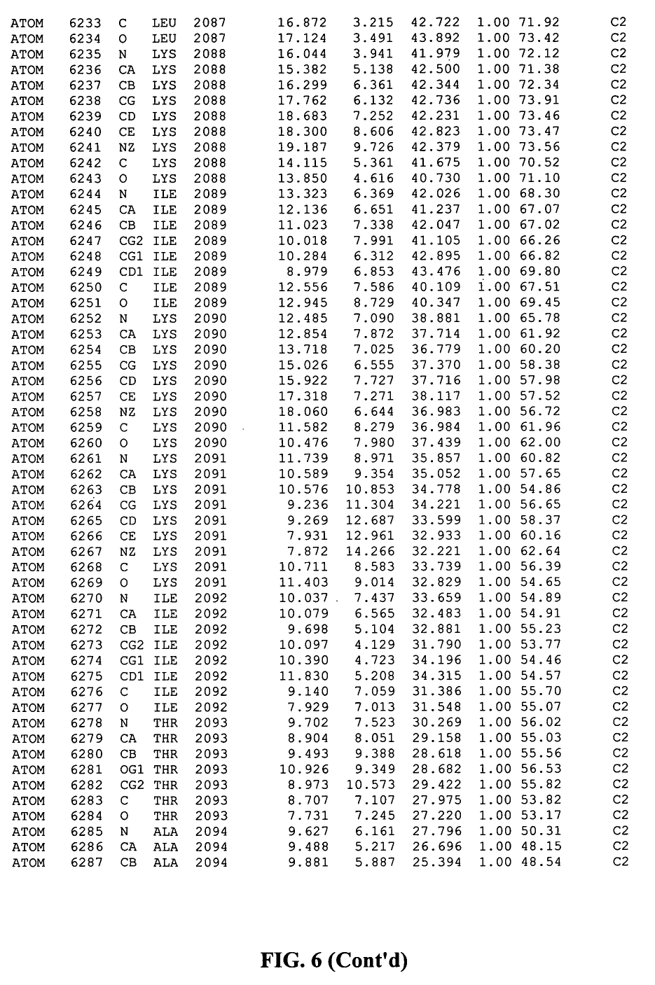

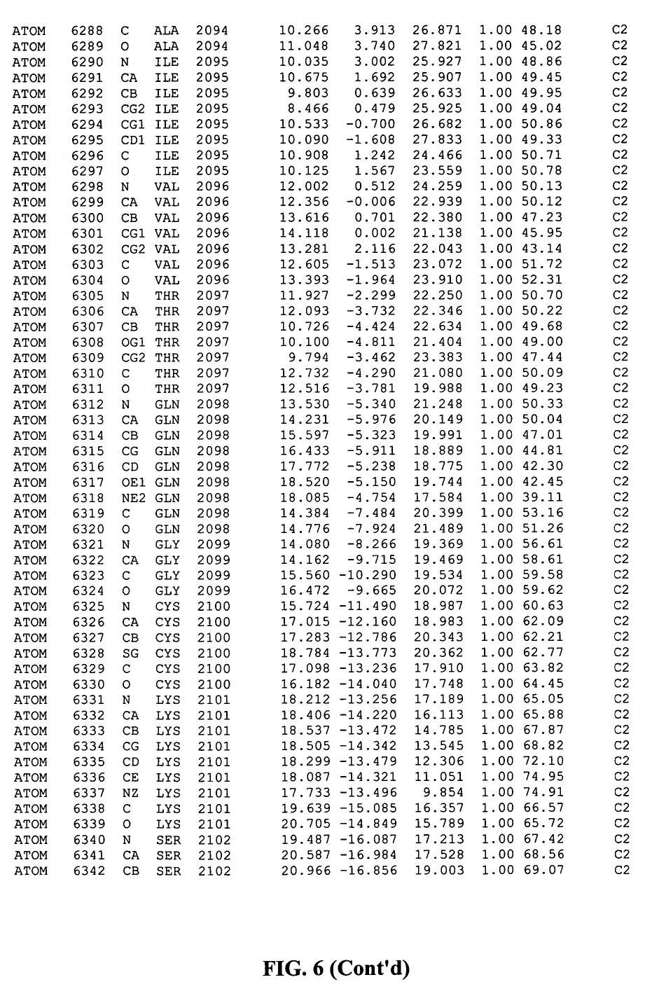

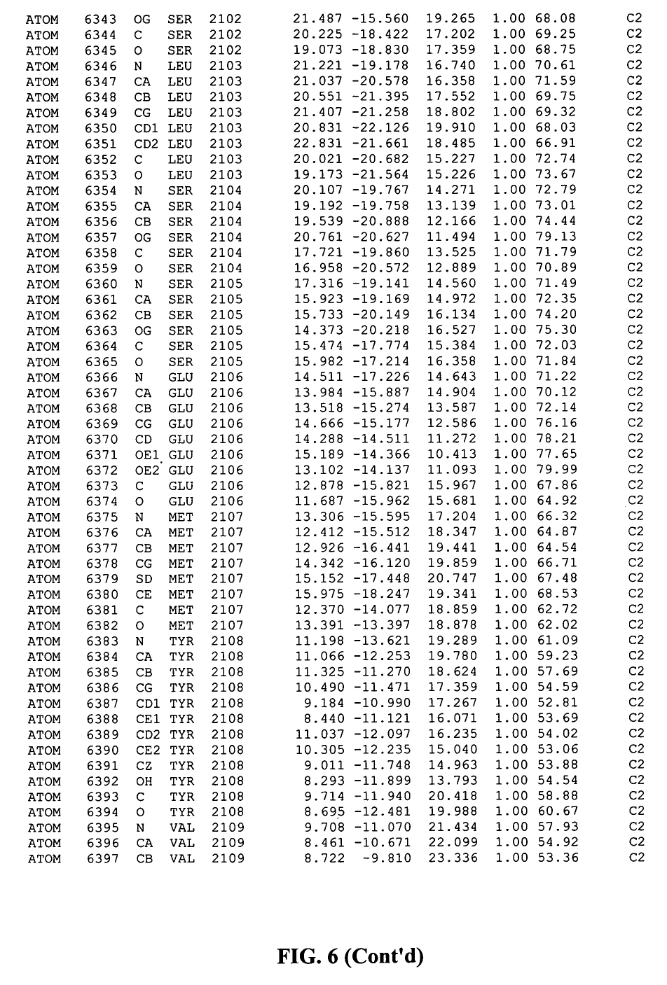

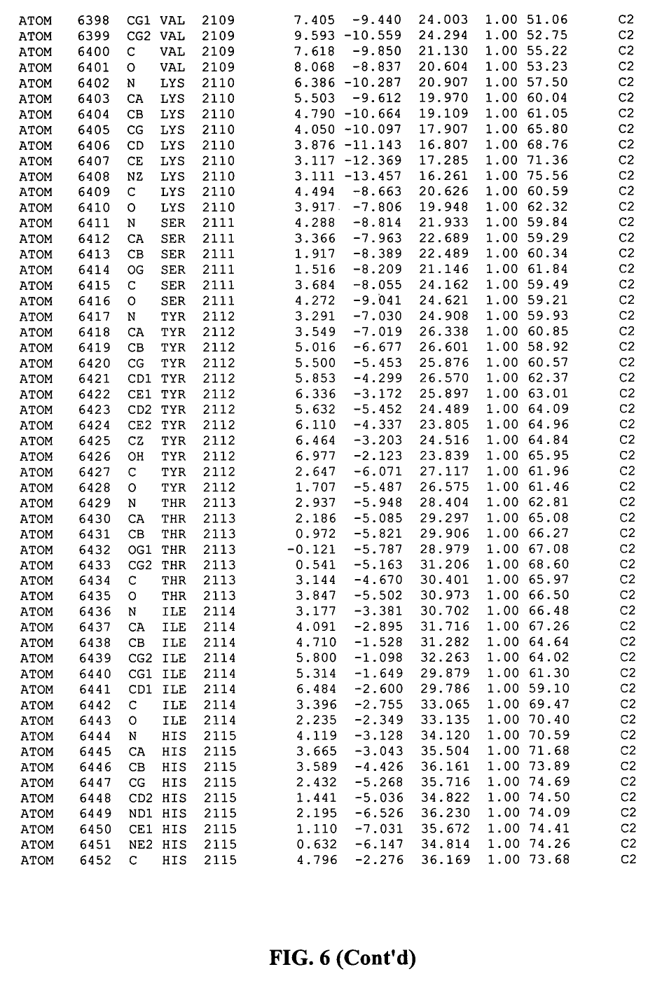

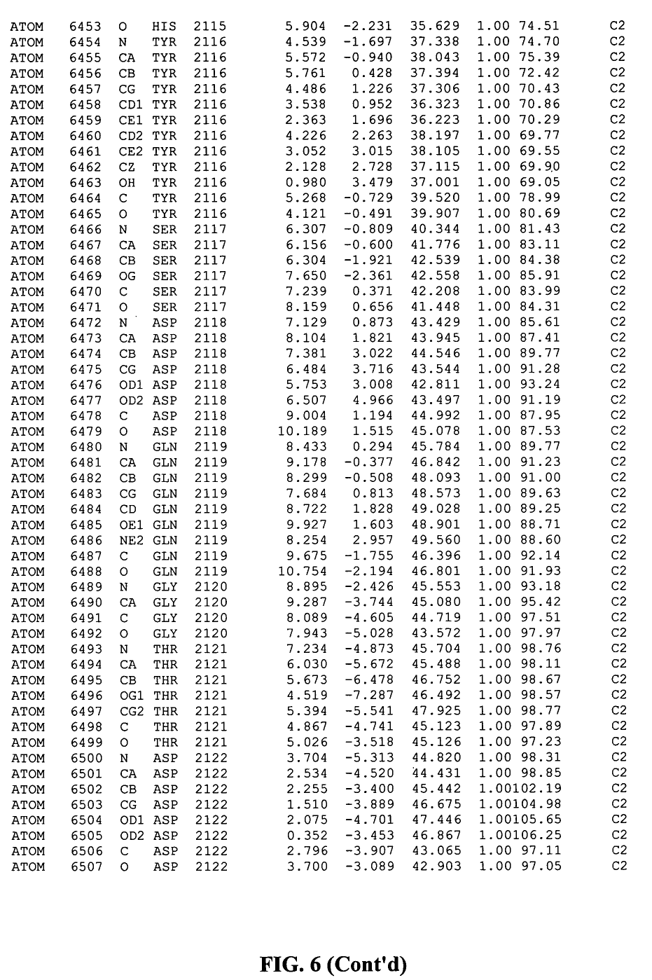

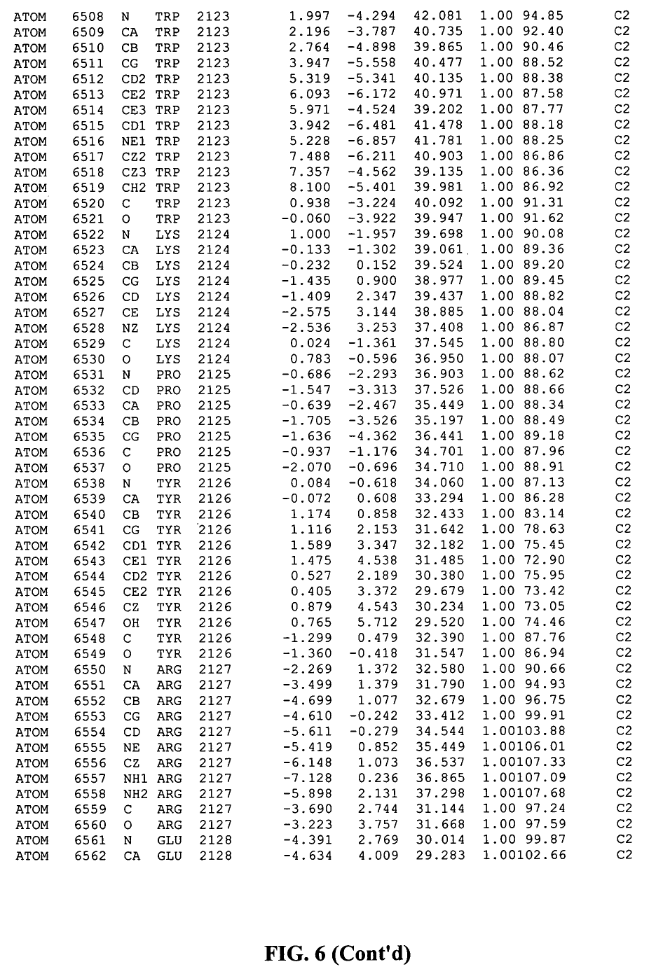

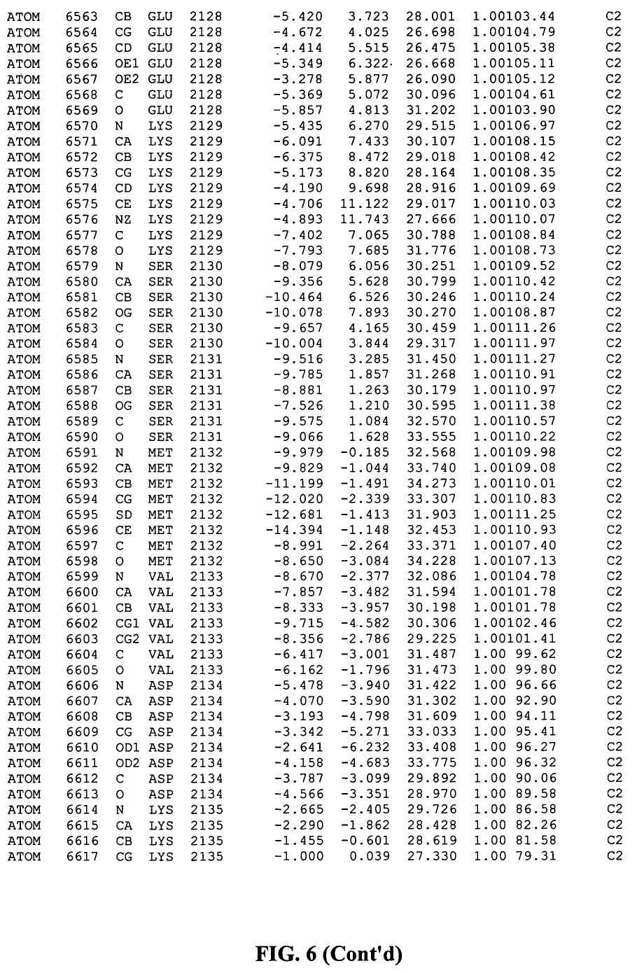

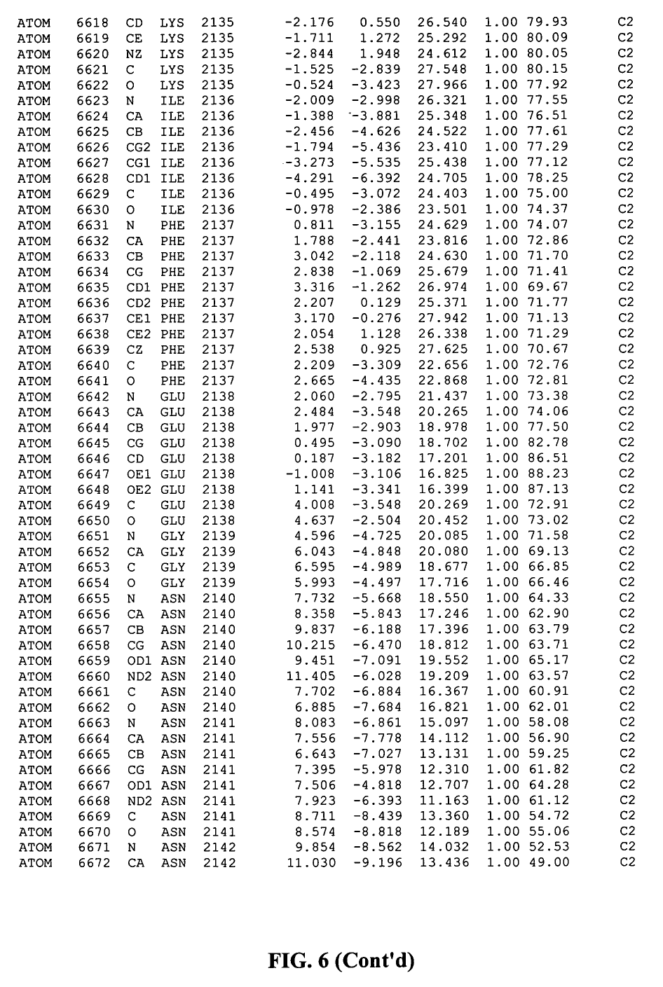

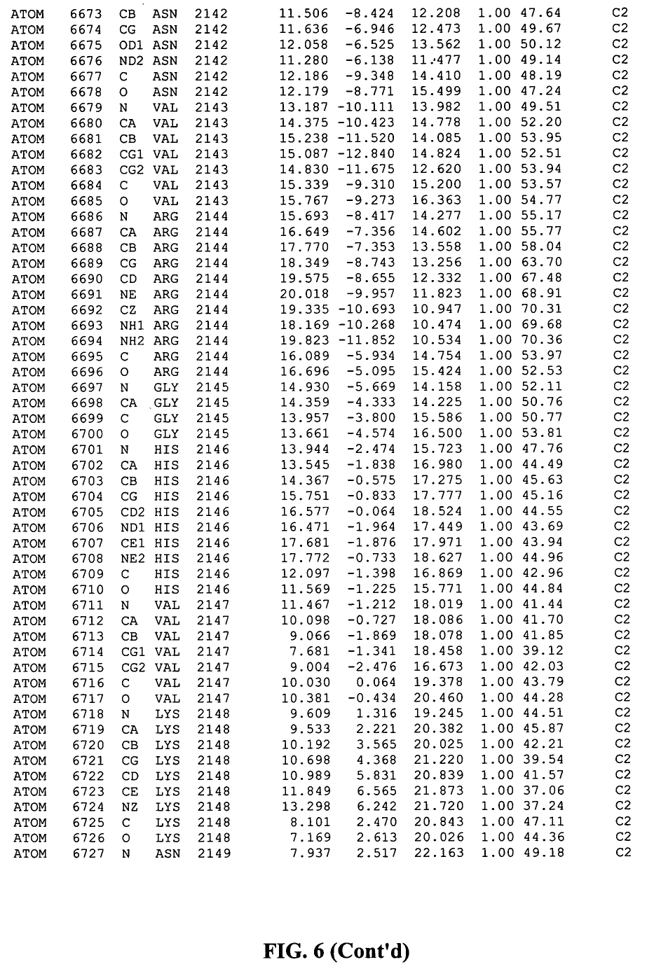

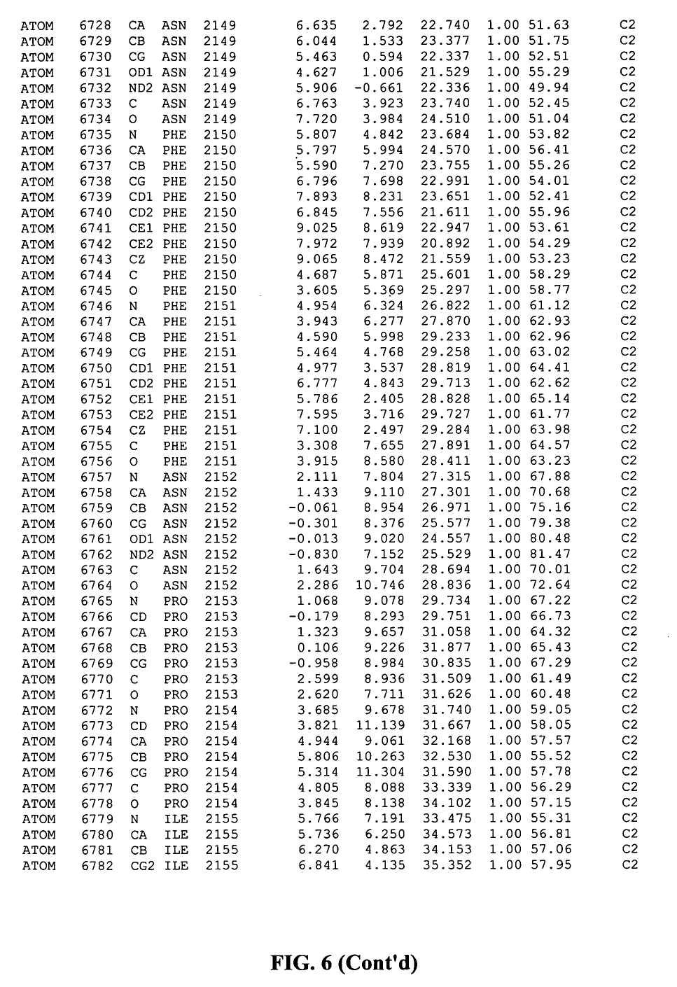

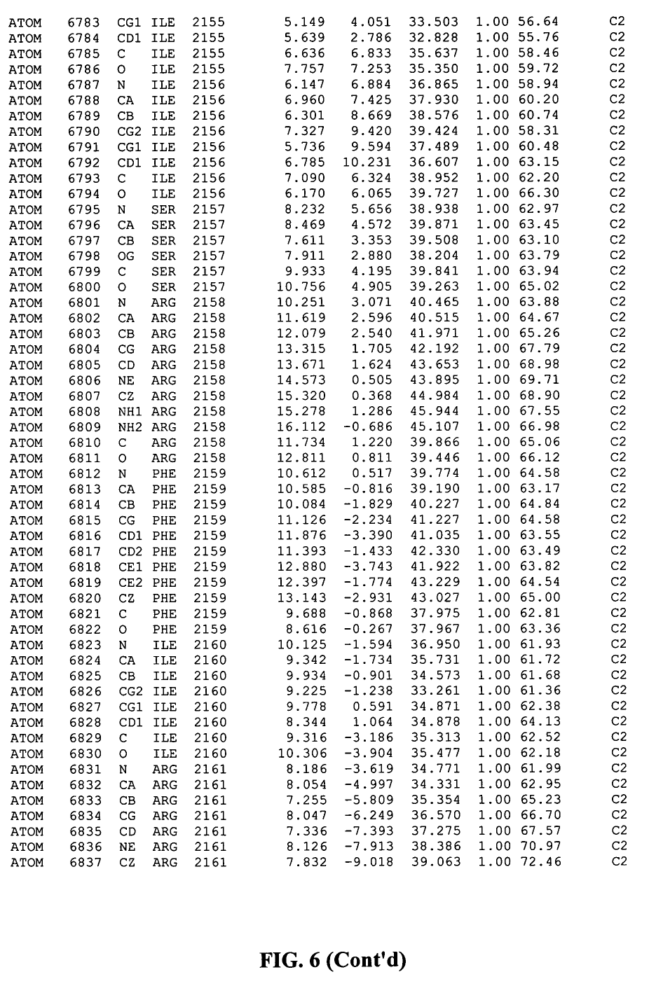

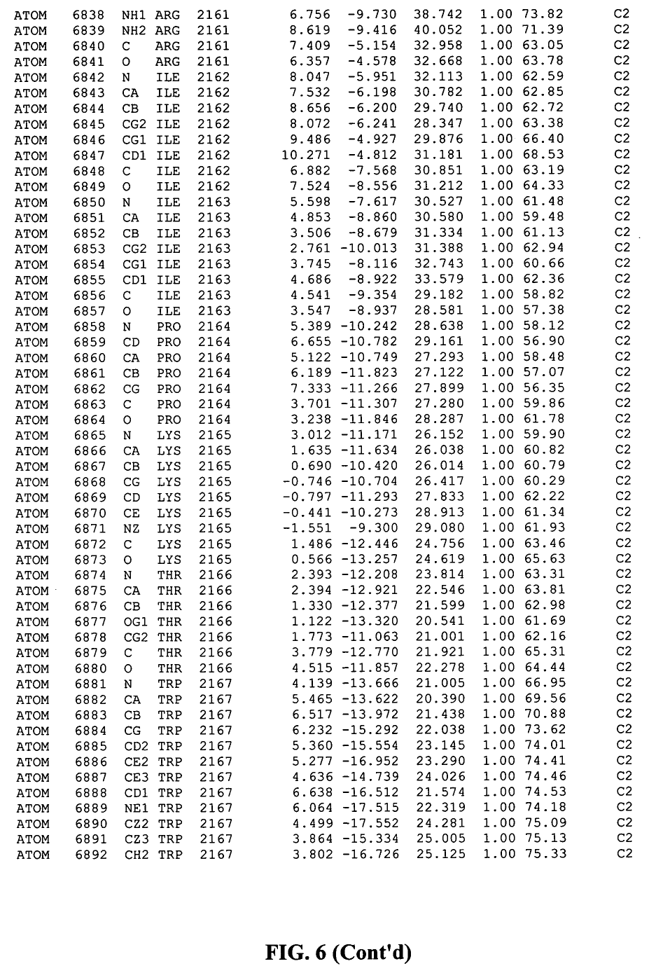

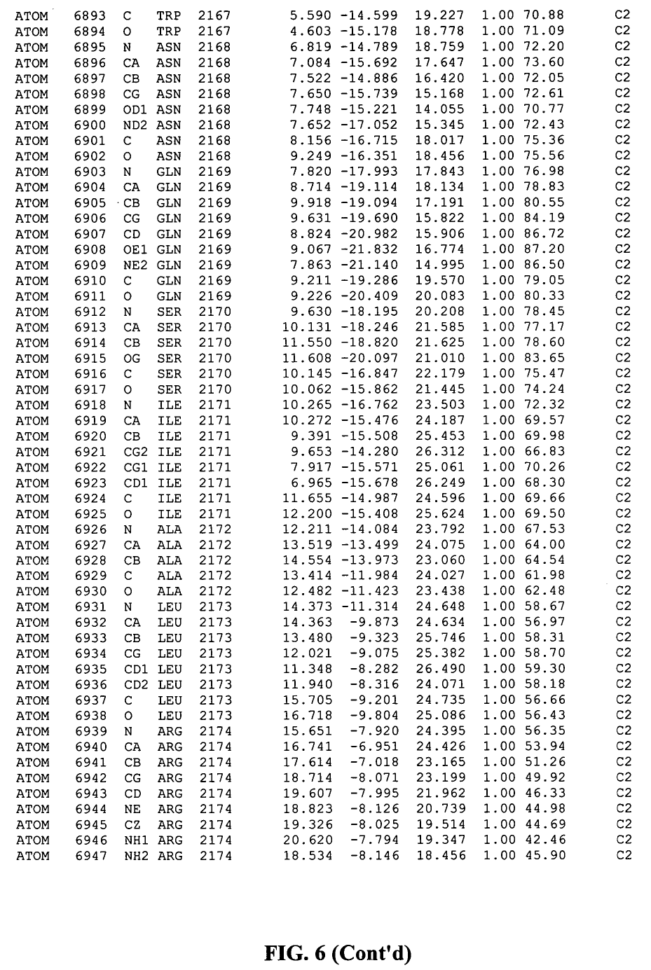

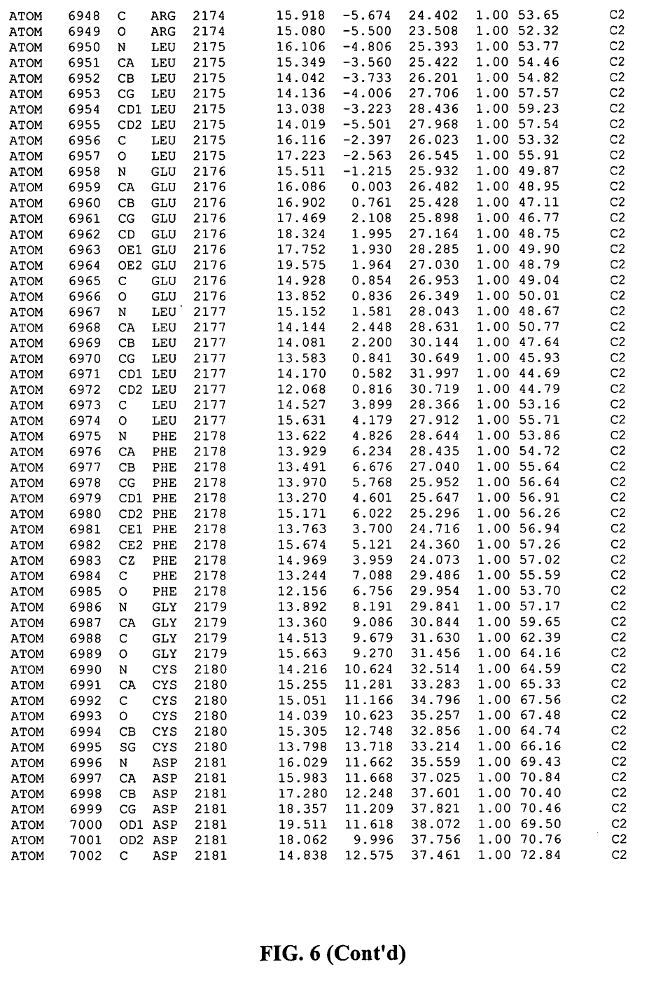

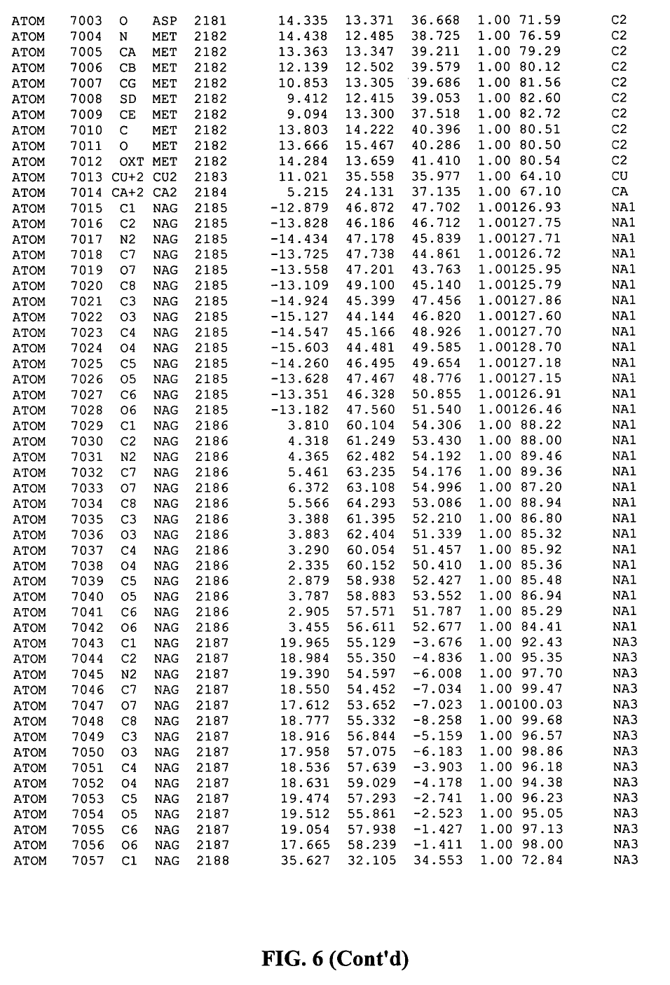

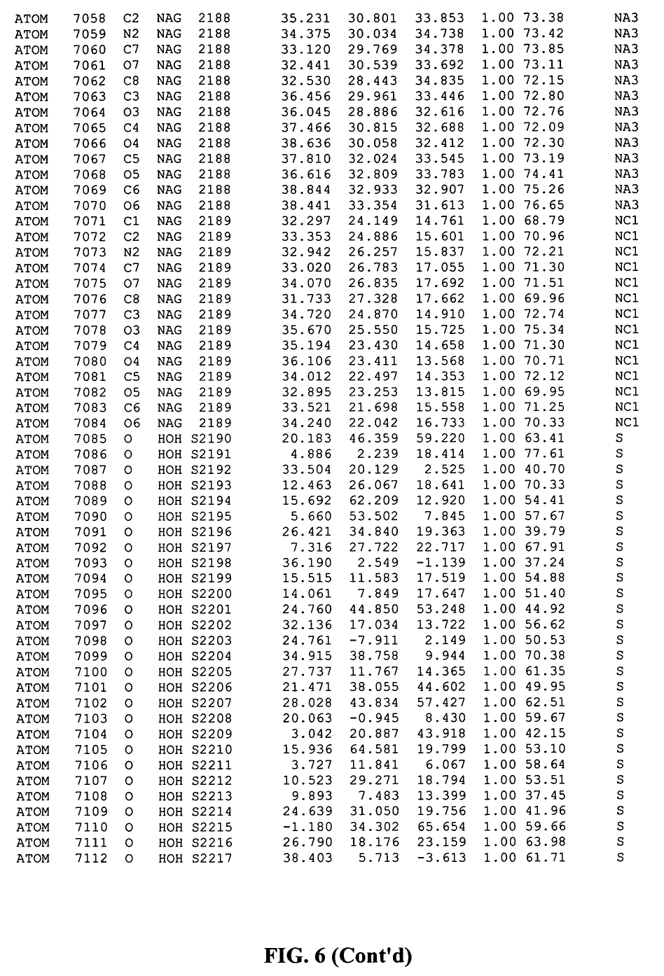

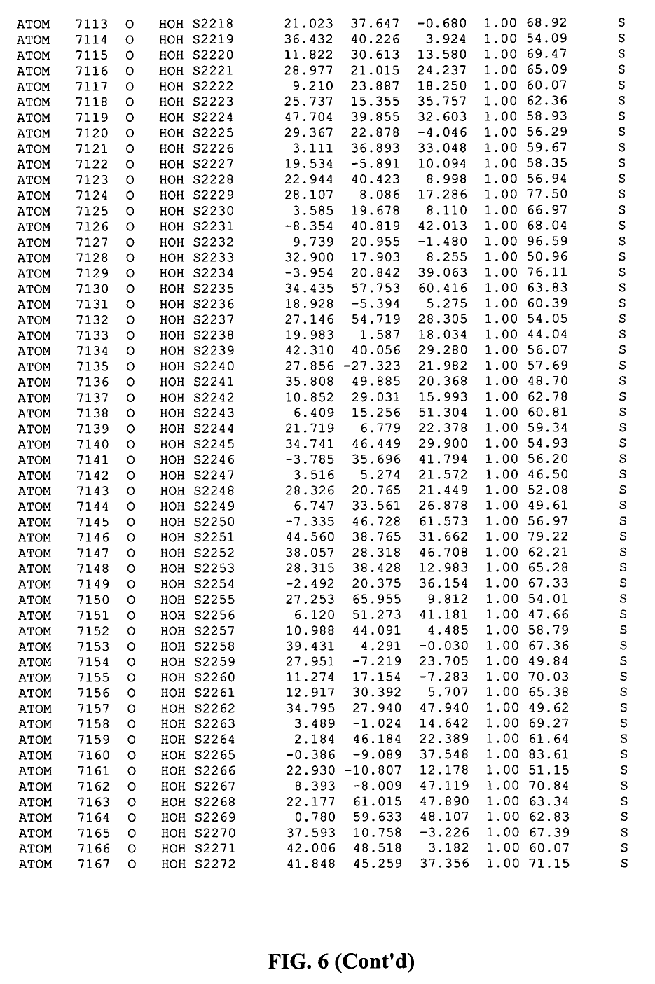

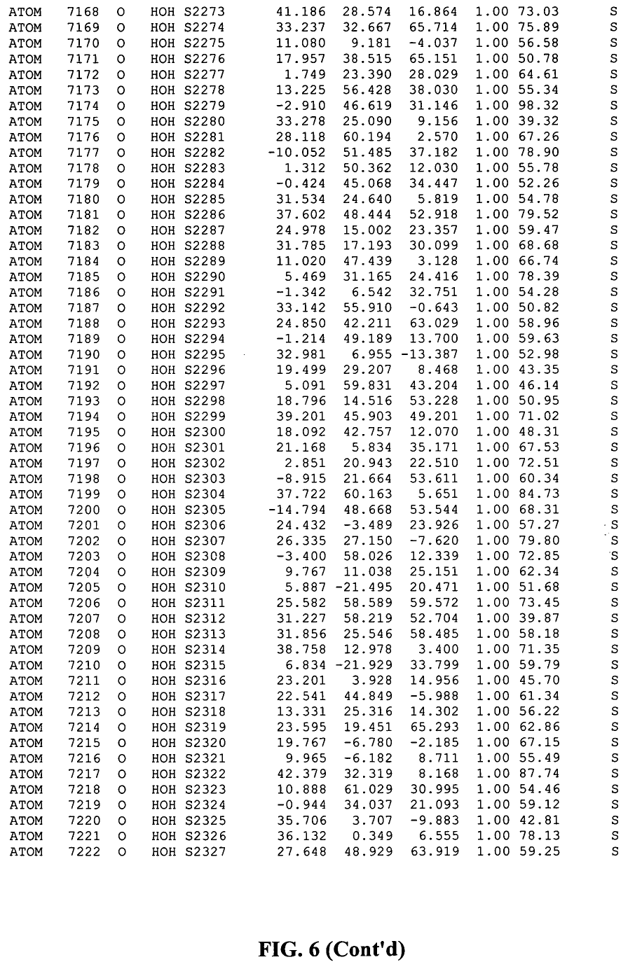

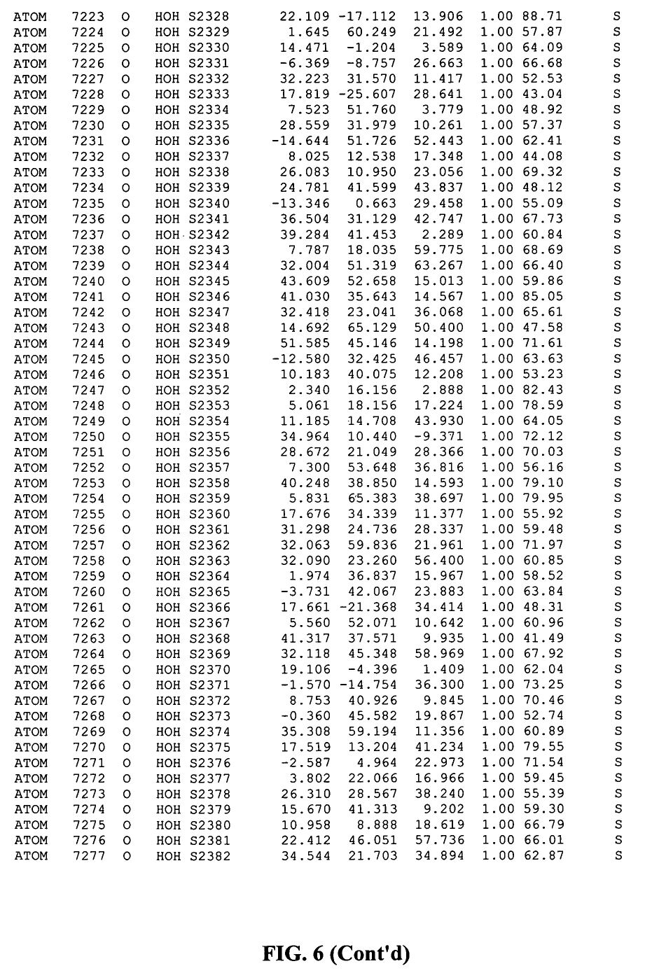

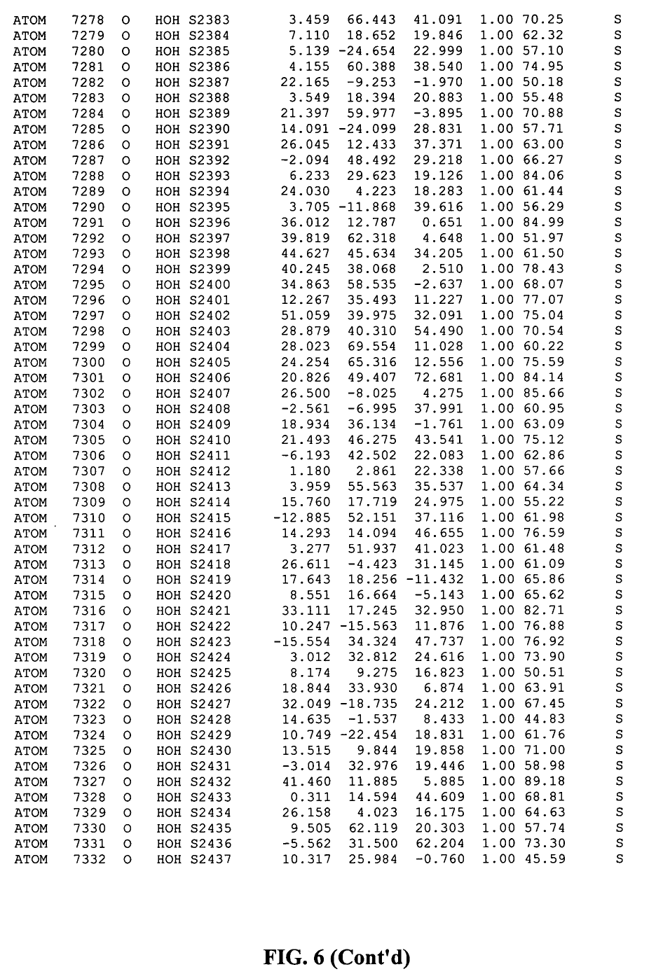

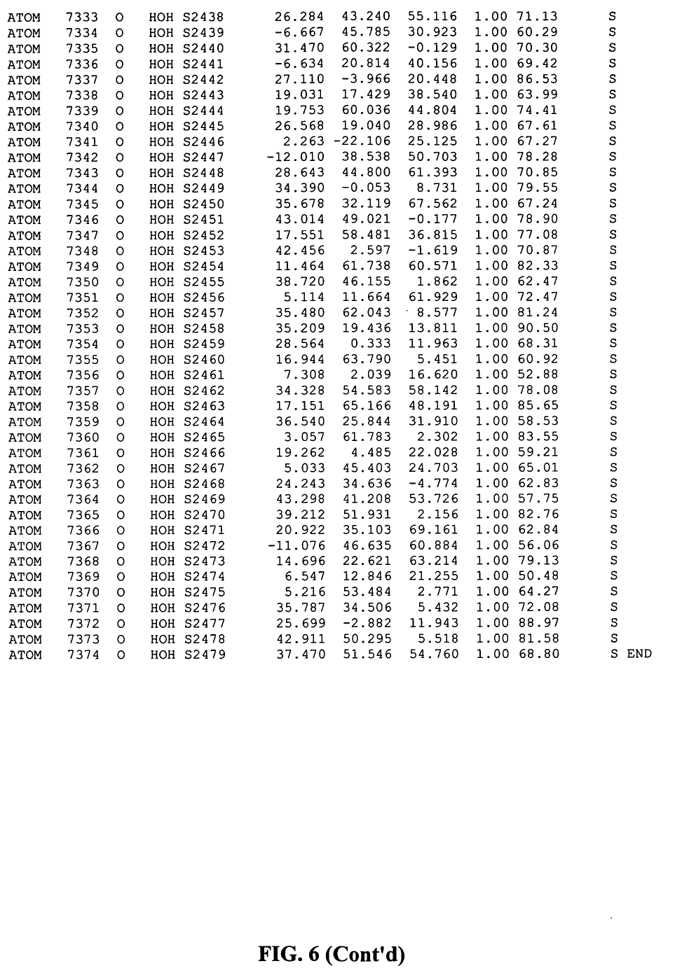

FIG. 6 provides the atomic coordinates that have been determined for bovine factor Vai.

What is claimed is:

1. A crystal of bovine factor Vai comprising atoms spatially arranged as represented by the structure coordinates listed in FIG. 6.

2. The crystal of claim 1 wherein the crystal comprises a 268 amino acid heavy chain and a 601 amino acid light chain, 333 water molecules, a calcium atom, a copper atom, and 5 carbohydrate molecules.

3. The crystal of claim 1 wherein the carbohydrate sites at 197, 211, 1662, 1811 and 1969 are glycosylated.

4. A bovine factor Vai crystal comprising an orthorhombic unit cell having dimensions of a,b, and c: wherein a is about 63.37 Å, b is about 86.56 Å, and c is about 229.20 Å.

5. (canceled)

6. A crystal of bovine factor Vai prepared by a method comprising:
i) suspending bovine factor Vai in a buffer at about physiological pH;
ii) crystallizing said factor by vapor diffusion using hanging or sitting drops at about 12° C.;
iii) growing crystals of factor Vai in a mother liquor optionally containing a heavy metal complex; and;
iv) cryoprotecting the crystals by slow dehydration.

7. The crystal of claim 6 wherein the dehydration is by rapidly dipping the crystals in an increasing concentration of about 2-16% ethylene glycol in steps of about 2%.

8. The crystal of claim 6 wherein the vapor diffusion sitting drop is against 200 mM MgCl₂, 16%PEG 3350 (pH 5.0).

9. The crystal of claim 6 wherein the heavy metal complex is selected from the group consisting of 10 mM tetrakismercuroxymethane (TAMM), 10 mM ethylmercury (EtHg) and 2.5 mM lead acetate (PbAc).

10. A method for crystallizing bovine factor Vai comprising growing bovine factor Vai crystals from a vapor diffusion drop preparation in a mother liquor optionally containing a lead or mercury complex ion for a period of about 5-21 days to obtain an isomorphous heavy metal crystal of bovine factor Vai.

11. A method of designing a potential anti-clotting compound for the prevention or treatment of a bleeding condition, comprising:
i) obtaining crystals of the bovine factor Vai wherein the crystals have the space group P2₁P2₁P2₁ with approximate a−63.37 Å and approximate b=86.56 Å and approximate c=229.20 Å such that the crystallized structure of factor Vai can be determined to a resolution of about 2.8 Å or below;
ii) evaluating the three dimensional structure of the crystallized factor Vai;
iii) synthesizing the potential interacting compounds based on the three-dimensional crystal structure of the crystallized Vai factor;
iv) contacting a molecule selected from prothrombin, factor Xa and combinations thereof with the potential anti-coagulation compound; and
v) assaying the potential anti-coagulation compound to determine the effect on thrombin generation when the anti-clotting compound is contacted with a prothrombinase complex wherein a decrease in rate of thrombin generation indicates potential use of said compound as an anti-coagulation factor.

12. The method of claim 11 wherein the crystallization is in hanging drops using a vapor diffusion method.

13. The method of claim 11 wherein the anti-coagulation compound is a peptide, lipid or polypeptide.

14. The method of claim 11 wherein the crystals have the three-dimensional crystal structure of factor Vai as shown in FIG. 1B.

15. A method for designing a candidate compound useful as a prothrombinase complex inhibitor or enhancer, comprising:
i) utilizing the three dimensional structure of crystallized factor Vai that is defined by the atomic coordinates in FIG. 6; and
ii) designing a candidate inhibitor compound based on the three-dimensional crystal structure of crystallized factor Vai for binding to Va or to the prothrombinase complex; and
iii) testing said candidate compound to determine inhibitor or enhancer properties.

16. The method of claim 15 wherein the candidate compound is selected from the group consisting of a peptide, polypeptide and a lipid.

17. A model of the structure of factor Vai, comprising a data set embodying the structure of the crystal of claim 1.

18. A method of identifying a molecule that is an inhibitor of prothrombinase complex or factor Va, comprising:
i) providing a model of the structure of factor Vai;
ii) identifying interactions of candidate molecules with selected domains of the model;
iii) selecting a candidate molecule that interacts with the selected domains; and
iv) testing the candidate molecule to confirm identification as an inhibitor or enhancer.

19. A computer assisted model for designing an inhibitor or an enhancer of prothrombinase complex, comprising:
i) providing a computer modeling application with a set of structural coordinates of a molecule or molecular complex comprising at least a part of a factor Vai proposed binding site;
ii) loading the computer modeling apparatus with a set of structural coordinates of a chemical structure;
iii) evaluating potential binding interactions between the chemical structure and the Vai binding site;
iv) structurally modifying the chemical structure to yield a set of structural coordinates for a modified chemical entity; and
v) determining inhibitor or enhancer properties of the modified chemical entity.

20. A computer readable storage medium comprising data storage material encoded with X-ray crystallographic atomic coordinates or at least a portion of said coordinates as set forth in FIG. 6.
